(19) United States
(12) Patent Application Publication
(10) Pub. No.: US 2005/0158308 A1
(43) Pub. Date: Jul. 21, 2005

(54) HEH4 molecules and uses thereof

(75) Inventors: William Scott Simonet, Thousand Oaks, CA (US); Marlese Pisegna, Thousand Oaks, CA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Amgen Inc. A Corporation of the State of Delaware

(21) Appl. No.: 11/015,205

(22) Filed: Dec. 17, 2004

Related U.S. Application Data
(60) Provisional application No. 60/532,154, filed on Dec. 22, 2003.

(51) Int. Cl. A61K 39/395

(52) U.S. Cl. 424/141.1; 424/146.1

(57) ABSTRACT

The present invention provides HEH4 polypeptides and nucleic acid molecules encoding the same. The invention also provides selective binding agents, vectors, host cells, and methods for producing HEH4 polypeptides. The invention further provides pharmaceutical compositions and methods for the diagnosis, treatment, amelioration, and/or prevention of diseases, disorders, and conditions associated with HEH4 polypeptides.

This application claims the benefit of priority from U.S. Provisional App. No. 60/532,154, filed Dec. 22, 2003, the disclosure of which is explicitly incorporated by reference herein.

### FIELD OF THE INVENTION

The present invention relates to HEH4 polypeptides and nucleic acid molecules encoding the same. The invention also relates to selective binding agents, vectors, host cells, and methods for producing HEH4 polypeptides. The invention further relates to pharmaceutical compositions and methods for the diagnosis, treatment, amelioration, or prevention of diseases, disorders, and conditions associated with HEH4 polypeptides.

### BACKGROUND OF THE INVENTION

Osteoporosis and osteopenia are the most common metabolic bone diseases in the developed countries of the world. These disorders are characterized by reduced bone mass, bone thinning and weakening, and an increased incidence of fractures. Senile osteoporosis describes the condition in older patients of both sexes. Post-menopausal osteoporosis describes the condition in women, wherein osteoporosis is associated with the decreased production of estrogen following menopause.

The early stage of the disease, referred to as osteopenia, is characterized by decreased bone mineral density (BMD), i.e., 1 to 2.5 standard deviations below normal peak BMD. Osteoporosis is defined as having a BMD greater than 2.5 standard deviations below normal peak BMD.

The incidence of osteoporotic fractures increases with age, is higher in whites than in blacks, and is higher in women than in men. It has been difficult to obtain precise figures as to the true prevalence of osteoporosis, since most osteoporotic fractures do not require hospitalization. However, since nearly all individuals suffering osteoporotic hip fractures must be hospitalized, a reliable estimate of the number of persons suffering from such fractures in the United States each year has been set at approximately 175,000 individuals. Most osteoporotic hip fractures require surgical intervention, and despite improvements in surgical techniques and anesthesiology, a 15% to 20% increase in mortality is observed following such fractures. In addition, less than one-third of all individuals suffering such fractures are restored to their pre-fracture functional state within a year of incidence, and most patients require some sort of ambulatory support or institutional care. Current estimates indicate that an individual suffering from an osteoporotic hip fracture will require $40,000 in annual medical expenditures.

Bone is constantly undergoing remodeling. This remodeling is carried out by two types of bone cells: osteoclasts, which resorb (or degrade) bone, and osteoblasts, which deposit new bone. While several approved therapeutics exist for the treatment of “low bone mineral density”-related diseases or disorders, such as osteoporosis, all of these therapeutic agents are anti-resorptive compounds that slow the rate of bone degradation by their ability to decrease osteoclast mediated bone resorption. Although therapeutically desirable, at present there exists no approved therapy in which an anabolic agent is used to stimulate osteoblast-mediated formation of new bone.

In articular cartilage, the chondrocyte is thought to degrade as well as synthesize new tissue, thereby helping to maintain the functional integrity of the cartilage. Bone and the synovial membrane are thought to also play important roles in maintaining the functional integrity of cartilage and thus may play important roles in the development of articular cartilage-related diseases such as osteoarthritis (Poole, 1999, Frontiers in Bioscience 4:D662-D670; Hough, “Pathology of Osteoarthritis,” in: Arthritis and Allied Conditions: A Textbook of Rheumatology 2167-94 (Koopman, ed. 2001)). There are very limited therapeutic agents available for the treatment of articular cartilage-related diseases, most notably osteoarthritis. Most of the therapeutic agents that are available only control pain and neither halt the destruction of articular cartilage nor bring about articular cartilage repair.

Osteoarthritis is a complex disorder that is probably a group of overlapping distinct diseases of the articlular cartilage, joints, and bone tissue. These diseases result from mechanical or biological factors that destabilize the normal coupling of synthesis and degradation of the extracellular matrix in articular cartilage and subchondral bone (Reginato et al., 2002, Arthritis Res. 4:337-45).

Genes or proteins that play important roles in the biology of bone or articular cartilage in adults are themselves potential therapeutic agents for treating bone or articular cartilage related diseases, such as osteoporosis, osteoarthritis or degenerative joint disease, and rheumatoid arthritis. Agonists or antagonists of genes or proteins that play important roles in the biology of bone or articular cartilage in adults are also potential therapeutic agents for treating bone or articular cartilage related diseases, such as osteoporosis, osteoarthritis or degenerative joint disease, and rheumatoid arthritis.

### SUMMARY OF THE INVENTION

The present invention relates to novel HEH4 nucleic acid molecules and encoded polypeptides.

The invention provides for an isolated nucleic acid molecule comprising a nucleotide sequence:
(a) as set forth in either SEQ ID NO: 1 or SEQ ID NO: 3;
(b) encoding a polypeptide as set forth in any of SEQ ID NO: 2, SEQ ID NO: 4, or SEQ ID NO: 5;
(c) that hybridizes under at least moderately stringent conditions to the complement of the nucleotide sequence of either (a) or (b), wherein the nucleic acid molecule encodes a polypeptide having an activity of the polypeptide set forth in any of SEQ ID NO: 2, SEQ ID NO: 4, or SEQ ID NO: 5; or
(d) complementary to the nucleotide sequence of any of (a)-(c).

The invention also provides for an isolated nucleic acid molecule comprising:
(a) a nucleotide sequence encoding a polypeptide that is at least about 70 percent identical to a polypeptide as set forth in any of SEQ ID NO: 2, SEQ ID NO: 4, or SEQ ID NO: 5, wherein the encoded polypeptide has an activity of the polypeptide set forth in any of SEQ ID NO: 2, SEQ ID NO: 4, or SEQ ID NO: 5;
(b) a nucleotide sequence encoding an allelic variant or splice variant of a nucleotide sequence as set forth in either SEQ ID NO: 1 or SEQ ID NO: 3, or the nucleotide sequence of (a);
(c) a region of a nucleotide sequence of either SEQ ID NO: 1 or SEQ ID NO: 3, or the nucleotide sequence of either (a) or (b), encoding a polypeptide fragment of at least about 25 amino acid residues, wherein the polypeptide fragment has an activity of the polypeptide set forth in any of SEQ ID NO: 2, SEQ ID NO: 4, or SEQ ID NO: 5, or is antigenic;
(d) a region of a nucleotide sequence of either SEQ ID NO: 1 or SEQ ID NO: 3, or the nucleotide sequence of any of (a)-(c), comprising a fragment of at least about 16 nucleotides;
(e) a nucleotide sequence that hybridizes under at least moderately stringent conditions to the complement of the nucleotide sequence of any of (a)-(d), wherein the nucleic acid molecule encodes a polypeptide having an activity of the polypeptide set forth in any of SEQ ID NO: 2, SEQ ID NO: 4, or SEQ ID NO: 5; or
(f) a nucleotide sequence complementary to the nucleotide sequence of any of (a)-(e).

The invention further provides for an isolated nucleic acid molecule comprising a nucleotide sequence:
(a) encoding a polypeptide as set forth in any of SEQ ID NO: 2, SEQ ID NO: 4, or SEQ ID NO: 5 with at least one conservative amino acid substitution, wherein the encoded polypeptide has an activity of the polypeptide set forth in any of SEQ ID NO: 2, SEQ ID NO: 4, or SEQ ID NO: 5;
(b) encoding a polypeptide as set forth in any of SEQ ID NO: 2, SEQ ID NO: 4, or SEQ ID NO: 5 with at least one amino acid insertion, wherein the encoded polypeptide has an activity of the polypeptide set forth in any of SEQ ID NO: 2, SEQ ID NO: 4, or SEQ ID NO: 5;
(c) encoding a polypeptide as set forth in any of SEQ ID NO: 2, SEQ ID NO: 4, or SEQ ID NO: 5 with at least one amino acid deletion, wherein the encoded polypeptide has an activity of the polypeptide set forth in any of SEQ ID NO: 2, SEQ ID NO: 4, or SEQ ID NO: 5;
(d) encoding a polypeptide as set forth in any of SEQ ID NO: 2, SEQ ID NO: 4, or SEQ ID NO: 5 that has a C- and/or N- terminal truncation, wherein the encoded polypeptide has an activity of the polypeptide set forth in any of SEQ ID NO: 2, SEQ ID NO: 4, or SEQ ID NO: 5;
(e) encoding a polypeptide as set forth in any of SEQ ID NO: 2, SEQ ID NO: 4, or SEQ ID NO: 5 with at least one modification that is an amino acid substitution, amino acid insertion, amino acid deletion, C-terminal truncation, or N-terminal truncation, wherein the encoded polypeptide has an activity of the polypeptide set forth in any of SEQ ID NO: 2, SEQ ID NO: 4, or SEQ ID NO: 5;
(f) of any of (a)-(e) comprising a fragment of at least about 16 nucleotides;
(g) that hybridizes under at least moderately stringent conditions to the complement of the nucleotide sequence of any of (a)-(f), wherein the nucleic acid molecule encodes a polypeptide having an activity of the polypeptide set forth in any of SEQ ID NO: 2, SEQ ID NO: 4, or SEQ ID NO: 5; or
(h) complementary to the nucleotide sequence of any of (a)-(g).

The present invention provides for an isolated polypeptide comprising an amino acid sequence as set forth in any of SEQ ID NO: 2, SEQ ID NO: 4, or SEQ ID NO: 5.

The invention also provides for an isolated polypeptide comprising:
(a) an amino acid sequence for an ortholog of any of SEQ ID NO: 2, SEQ ID NO: 4, or SEQ ID NO: 5;
(b) an amino acid sequence which is at least about 70 percent identical to an amino acid sequence of any of SEQ ID NO: 2, SEQ ID NO: 4, or SEQ ID NO: 5, wherein the polypeptide has an activity of the polypeptide set forth in any of SEQ ID NO: 2, SEQ ID NO: 4, or SEQ ID NO: 5;
(c) a fragment of the amino acid sequence set forth in any of SEQ ID NO: 2, SEQ ID NO: 4, or SEQ ID NO: 5 comprising at least about 25 amino acid residues, wherein the fragment has an activity of the polypeptide set forth in any of SEQ ID NO: 2, SEQ ID NO: 4, or SEQ ID NO: 5, or is antigenic; or
(d) an amino acid sequence for an allelic variant or splice variant of the amino acid sequence as set forth in any of SEQ ID NO: 2, SEQ ID NO: 4, or SEQ ID NO: 5, or the amino acid sequence of either (a) or (b).

The invention further provides for an isolated polypeptide comprising an amino acid sequence as set forth in SEQ ID NO: 2, SEQ ID NO: 4, or SEQ ID NO: 5:
(a) with at least one conservative amino acid substitution;
(b) with at least one amino acid insertion;
(c) with at least one amino acid deletion;
(d) that has a C- and/or N- terminal truncation; or
(e) with at least one modification that is an amino acid substitution, amino acid insertion, amino acid deletion, C-terminal truncation, or N-terminal truncation;
wherein the polypeptide has an activity of the polypeptide set forth in any of SEQ ID NO: 2, SEQ ID NO: 4, or SEQ ID NO: 5.

The present invention provides for an expression vector comprising the isolated nucleic acid molecules as set forth herein, recombinant host cells comprising the recombinant nucleic acid molecules as set forth herein, and a method of producing a HEH4 polypeptide comprising culturing the host cells and optionally isolating the polypeptide so produced.

The invention also provides fusion polypeptides comprising at least one HEH4 polypeptide fused to a heterologous amino acid sequence. The invention further provides derivatives of the HEH4 polypeptides of the present invention.

The present invention provides selective binding agents capable of specifically binding at least one polypeptide comprising the amino acid sequence as set forth in any of SEQ ID NO: 2, SEQ ID NO: 4, or SEQ ID NO: 5.

The selective binding agents of the present invention can be antibodies, or fragments thereof, including, but not limited to: murine antibodies, humanized antibodies, human antibodies, polyclonal antibodies, monoclonal antibodies, chimeric antibodies, CDR-grafted antibodies, antiidiotypic antibodies, and variable region fragments (such as Fab or a Fab′ fragments). The selective binding agents of the present invention include selective binding agents or fragments thereof having at least one complementarity-determining region with specificity for a polypeptide comprising the amino acid sequence as set forth in any of SEQ ID NO: 2, SEQ ID NO: 4, or SEQ ID NO: 5.

The selective binding agents of the invention can optionally be bound to a detectable label.

Also provided are selective binding agents that are capable of antagonizing HEH4 biological activity.

The present invention also provides pharmaceutical compositions comprising the polypeptides or selective binding agents of the invention and one or more pharmaceutically acceptable formulation agents are also encompassed by the invention.

The formulation agent can be a suitable carrier, adjuvant, solubilizer, stabilizer, or anti-oxidant. HEH4 polypeptides or selective binding agents can be covalently modified with a water-soluble polymer, such as polyethylene glycol and dextran. The pharmaceutical compositions of the present invention are used to provide therapeutically effective amounts of the HEH4 polypeptides or selective binding agents of the present invention. The present invention also provides methods for the manufacture of a medicament for the treatment of HEH4-related diseases, conditions, or disorders.

The present invention also provides methods of diagnosing in an animal a HEH4-related disease, condition, or disorder, or a susceptibility to a HEH4-related disease, condition, or disorder, comprising determining the presence or amount of expression of a HEH4 polypeptide and diagnosing the HEH4-related disease, condition, or disorder, or susceptibility to a HEH4-related disease, condition, or disorder, based on the presence or amount of expression of the HEH4 polypeptide. In preferred methods of diagnosing a HEH4-related disease, condition, or disorder, or a susceptibility to a HEH4-related disease, condition, or disorder, the animal is a mammal. In even more preferred methods the animal is a human.

The present invention provides a method for treating, preventing, or ameliorating a bone-related disease, condition, or disorder comprising administering to a patient an effective amount of a selective binding agent or fragment thereof that specifically binds a polypeptide comprising an amino acid sequence as set forth in any of SEQ ID NO: 2, SEQ ID NO: 4, or SEQ ID NO: 5.

The present invention also provides a method for treating, preventing, or ameliorating a bone-related disease, condition, or disorder comprising administering to a patient an effective amount of a selective binding agent or fragment thereof that specifically binds a polypeptide comprising:
(a) an amino acid sequence for an ortholog of any of SEQ ID NO: 2, SEQ ID NO: 4, or SEQ ID NO: 5;
(b) an amino acid sequence that is at least about 70 percent identical to the amino acid sequence of any of SEQ ID NO: 2, SEQ ID NO: 4, or SEQ ID NO: 5, wherein the polypeptide has an activity of the polypeptide set forth any of SEQ ID NO: 2, SEQ ID NO: 4, or SEQ ID NO: 5;
(c) a fragment of the amino acid sequence set forth in any of SEQ ID NO: 2, SEQ ID NO: 4, or SEQ ID NO: 5 comprising at least about 25 amino acid residues, wherein the fragment has an activity of the polypeptide set forth in any of SEQ ID NO: 2, SEQ ID NO: 4, or SEQ ID NO: 5, or is antigenic; or
(d) an amino acid sequence for an allelic variant or splice variant of the amino acid sequence as set forth in any of SEQ ID NO: 2, SEQ ID NO: 4, or SEQ ID NO: 5, or the amino acid sequence of either (a) or (b).

The present invention further provides a method for treating, preventing, or ameliorating a bone-related disease, condition, or disorder comprising administering to a patient an effective amount of a selective binding agent or fragment thereof that specifically binds a polypeptide comprising an amino acid sequence as set forth in SEQ ID NO: 2, SEQ ID NO: 4, or SEQ ID NO: 5:
(a) with at least one conservative amino acid substitution;
(b) with at least one amino acid insertion;
(c) with at least one amino acid deletion;
(d) the amino acid sequence as set forth in any of SEQ ID NO: 2, SEQ ID NO: 4, or SEQ ID NO: 5 that has a C- and/or N- terminal truncation; or
(e) with at least one modification that is an amino acid substitution, amino acid insertion, amino acid deletion, C-terminal truncation, or N-terminal truncation;
wherein the polypeptide has an activity of the polypeptide set forth in any of SEQ ID NO: 2, SEQ ID NO: 4, or SEQ ID NO: 5.

The present invention also provides a method for treating, preventing, or ameliorating osteoporosis or osteopenia comprising administering to a patient an effective amount of a selective binding agent or fragment thereof that specifically binds a polypeptide comprising an amino acid sequence as set forth in SEQ ID NO: 4, wherein the selective binding agent antagonizes HEH4 polypeptide biological activity.

The present invention also provides a method for treating, preventing, or ameliorating osteoporosis or osteopenia comprising administering to a patient an effective amount of an antibody or fragment thereof that specifically binds a polypeptide comprising an amino acid sequence as set forth in SEQ ID NO: 4, wherein the antibody antagonizes HEH4 polypeptide biological activity.

The present invention also provides a method for treating, preventing, or ameliorating a cartilage-related disease, condition, or disorder comprising administering to a patient an effective amount of a selective binding agent or fragment thereof that specifically binds a polypeptide comprising an amino acid sequence as set forth in any of SEQ ID NO: 2, SEQ ID NO: 4, or SEQ ID NO: 5.

The present invention also provides a method for treating, preventing, or ameliorating a cartilage-related disease, condition, or disorder comprising administering to a patient an effective amount of a selective binding agent or fragment thereof that specifically binds a polypeptide comprising:
(a) an amino acid sequence for an ortholog of any of SEQ ID NO: 2, SEQ ID NO: 4, or SEQ ID NO: 5;
(b) an amino acid sequence that is at least about 70 percent identical to the amino acid sequence of any of SEQ ID NO: 2, SEQ ID NO: 4, or SEQ ID NO: 5, wherein the polypeptide has an activity of the polypeptide set forth any of SEQ ID NO: 2, SEQ ID NO: 4, or SEQ ID NO: 5;
(c) a fragment of the amino acid sequence set forth in any of SEQ ID NO: 2, SEQ ID NO: 4, or SEQ ID NO: 5 comprising at least about 25 amino acid residues, wherein the fragment has an activity of the polypeptide set forth in any of SEQ ID NO: 2, SEQ ID NO: 4, or SEQ ID NO: 5, or is antigenic; or
(d) an amino acid sequence for an allelic variant or splice variant of the amino acid sequence as set forth in any of SEQ ID NO: 2, SEQ ID NO: 4, or SEQ ID NO: 5, or the amino acid sequence of either (a) or (b).

The present invention also provides a method for treating, preventing, or ameliorating a cartilage-related disease, condition, or disorder comprising administering to a patient an effective amount of a selective binding agent or fragment thereof that specifically binds a polypeptide comprising an amino acid sequence as set forth in SEQ ID NO: 2, SEQ ID NO: 4, or SEQ ID NO: 5:
(a) with at least one conservative amino acid substitution;
(b) with at least one amino acid insertion;
(c) with at least one amino acid deletion;
(d) the amino acid sequence as set forth in any of SEQ ID NO: 2, SEQ ID NO: 4, or SEQ ID NO: 5 that has a C- and/or N- terminal truncation; or
(e) with at least one modification that is an amino acid substitution, amino acid insertion, amino acid deletion, C-terminal truncation, or N-terminal truncation;
wherein the polypeptide has an activity of the polypeptide set forth in any of SEQ ID NO: 2, SEQ ID NO: 4, or SEQ ID NO: 5.

The present invention also provides a method for treating, preventing, or ameliorating a bone-related disease, condition, or disorder comprising administering to a patient an effective amount of an agonist or antagonist of a polypeptide comprising an amino acid sequence as set forth in any of SEQ ID NO: 2, SEQ ID NO: 4, or SEQ ID NO: 5.

The present invention also provides a method for treating, preventing, or ameliorating a bone-related disease, condition, or disorder comprising administering to a patient an effective amount of an agonist or antagonist of a polypeptide comprising:
(a) an amino acid sequence for an ortholog of any of SEQ ID NO: 2, SEQ ID NO: 4, or SEQ ID NO: 5;
(b) an amino acid sequence that is at least about 70 percent identical to the amino acid sequence of any of SEQ ID NO: 2, SEQ ID NO: 4, or SEQ ID NO: 5, wherein the polypeptide has an activity of the polypeptide set forth any of SEQ ID NO: 2, SEQ ID NO: 4, or SEQ ID NO: 5;
(c) a fragment of the amino acid sequence set forth in any of SEQ ID NO: 2, SEQ ID NO: 4, or SEQ ID NO: 5 comprising at least about 25 amino acid residues, wherein the fragment has an activity of the polypeptide set forth in any of SEQ ID NO: 2, SEQ ID NO: 4, or SEQ ID NO: 5, or is antigenic; or
(d) an amino acid sequence for an allelic variant or splice variant of the amino acid sequence as set forth in any of SEQ ID NO: 2, SEQ ID NO: 4, or SEQ ID NO: 5, or the amino acid sequence of either (a) or (b).

The present invention also provides a method for treating, preventing, or ameliorating a bone-related disease, condition, or disorder comprising administering to a patient an effective amount of an agonist or antagonist of a polypeptide comprising an amino acid sequence as set forth in SEQ ID NO: 2, SEQ ID NO: 4, or SEQ ID NO: 5:
(a) with at least one conservative amino acid substitution;
(b) with at least one amino acid insertion;
(c) with at least one amino acid deletion;
(d) the amino acid sequence as set forth in any of SEQ ID NO: 2, SEQ ID NO: 4, or SEQ ID NO: 5 that has a C- and/or N- terminal truncation; or
(e) with at least one modification that is an amino acid substitution, amino acid insertion, amino acid deletion, C-terminal truncation, or N-terminal truncation;
wherein the polypeptide has an activity of the polypeptide set forth in any of SEQ ID NO: 2, SEQ ID NO: 4, or SEQ ID NO: 5.

The present invention also provides a method for treating, preventing, or ameliorating a cartilage-related disease, condition, or disorder comprising administering to a patient an effective amount of an agonist or antagonist of a polypeptide comprising an amino acid sequence as set forth in any of SEQ ID NO: 2, SEQ ID NO: 4, or SEQ ID NO: 5.

The present invention also provides a method for treating, preventing, or ameliorating a cartilage-related disease, condition, or disorder comprising administering to a patient an effective amount of an agonist or antagonist of a polypeptide comprising:
(a) an amino acid sequence for an ortholog of any of SEQ ID NO: 2, SEQ ID NO: 4, or SEQ ID NO: 5;
(b) an amino acid sequence that is at least about 70 percent identical to the amino acid sequence of any of SEQ ID NO: 2, SEQ ID NO: 4, or SEQ ID NO: 5, wherein the polypeptide has an activity of the polypeptide set forth any of SEQ ID NO: 2, SEQ ID NO: 4, or SEQ ID NO: 5;
(c) a fragment of the amino acid sequence set forth in any of SEQ ID NO: 2, SEQ ID NO: 4, or SEQ ID NO: 5 comprising at least about 25 amino acid residues, wherein the fragment has an activity of the polypeptide set forth in any of SEQ ID NO: 2, SEQ ID NO: 4, or SEQ ID NO: 5, or is antigenic; or
(d) an amino acid sequence for an allelic variant or splice variant of the amino acid sequence as set forth in any of SEQ ID NO: 2, SEQ ID NO: 4, or SEQ ID NO: 5, or the amino acid sequence of either (a) or (b).

The present invention also provides a method for treating, preventing, or ameliorating a cartilage-related disease, condition, or disorder comprising administering to a patient an effective amount of an agonist or antagonist of a polypeptide comprising an amino acid sequence as set forth in SEQ ID NO: 2, SEQ ID NO: 4, or SEQ ID NO: 5:
(a) with at least one conservative amino acid substitution;
(b) with at least one amino acid insertion;
(c) with at least one amino acid deletion;
(d) the amino acid sequence as set forth in any of SEQ ID NO: 2, SEQ ID NO: 4, or SEQ ID NO: 5 that has a C- and/or N- terminal truncation; or
(e) with at least one modification that is an amino acid substitution, amino acid insertion, amino acid deletion, C-terminal truncation, or N-terminal truncation;
wherein the polypeptide has an activity of the polypeptide set forth in any of SEQ ID NO: 2, SEQ ID NO: 4, or SEQ ID NO: 5.

The present invention also provides a method for treating, preventing, or ameliorating a bone-related disease, condition, or disorder comprising administering to a patient an isolated nucleic acid molecule comprising a nucleotide sequence:
(a) as set forth in either SEQ ID NO: 1 or SEQ ID NO: 3;
(b) encoding the polypeptide as set forth in any of SEQ ID NO: 2, SEQ ID NO: 4, or SEQ ID NO: 5;
(c) that hybridizes under at least moderately stringent conditions to the complement of the nucleotide sequence of either (a) or (b), wherein the nucleic acid molecule encodes polypeptide having an activity of the polypeptide set forth in any of SEQ ID NO: 2, SEQ ID NO: 4, or SEQ ID NO: 5; or
(d) complementary to the nucleotide sequence of any of (a)-(c).

The present invention also provides a method for treating, preventing, or ameliorating a bone-related disease, condition, or disorder comprising administering to a patient an isolated nucleic acid molecule comprising:
(a) a nucleotide sequence encoding a polypeptide that is at least about 70 percent identical to the polypeptide as set forth in any of SEQ ID NO: 2, SEQ ID NO: 4, or SEQ ID NO: 5, wherein the encoded polypeptide has an activity of the polypeptide set forth in any of SEQ ID NO: 2, SEQ ID NO: 4, or SEQ ID NO: 5;
(b) a nucleotide sequence encoding an allelic variant or splice variant of the nucleotide sequence as set forth in either SEQ ID NO: 1 or SEQ ID NO: 3, or the nucleotide sequence of (a);
(c) a region of the nucleotide sequence of any of SEQ ID NO: 1 or SEQ ID NO: 3, or the nucleotide sequence of (a) or (b) encoding a polypeptide fragment of at least about 25 amino acid residues, wherein the polypeptide fragment has an activity of the encoded polypeptide set forth in any of SEQ ID NO: 2, SEQ ID NO: 4, or SEQ ID NO: 5, or is antigenic;
(d) a region of the nucleotide sequence of any of SEQ ID NO: 1 or SEQ ID NO: 3, or the nucleotide sequence of any of (a)-(c) comprising a fragment of at least about 16 nucleotides;
(e) a nucleotide sequence that hybridizes under at least moderately stringent conditions to the complement of the nucleotide sequence of any of (a)-(d), wherein the nucleic acid molecule encodes a polypeptide having an activity of the polypeptide set forth in any of SEQ ID NO: 2, SEQ ID NO: 4, or SEQ ID NO: 5; or
(f) a nucleotide sequence complementary to the nucleotide sequence of any of (a)-(e).

The present invention also provides a method for treating, preventing, or ameliorating a bone-related disease, condition, or disorder comprising administering to a patient an isolated nucleic acid molecule comprising:
(a) encoding a polypeptide as set forth in any of SEQ ID NO: 2, SEQ ID NO: 4, or SEQ ID NO: 5 with at least one conservative amino acid substitution, wherein the encoded polypeptide has an activity of the polypeptide set forth in any of SEQ ID NO: 2, SEQ ID NO: 4, or SEQ ID NO: 5;
(b) encoding a polypeptide as set forth in any of SEQ ID NO: 2, SEQ ID NO: 4, or SEQ ID NO: 5 with at least one amino acid insertion, wherein the encoded polypeptide has an activity of the polypeptide set forth in any of SEQ ID NO: 2, SEQ ID NO: 4, or SEQ ID NO: 5;
(c) encoding a polypeptide as set forth in any of SEQ ID NO: 2, SEQ ID NO: 4, or SEQ ID NO: 5 with at least one amino acid deletion, wherein the encoded polypeptide has an activity of the polypeptide set forth in any of SEQ ID NO: 2, SEQ ID NO: 4, or SEQ ID NO: 5;
(d) encoding a polypeptide as set forth in any of SEQ ID NO: 2, SEQ ID NO: 4, or SEQ ID NO: 5 that has a C- and/or N- terminal truncation, wherein the encoded polypeptide has an activity of the polypeptide set forth in any of SEQ ID NO: 2, SEQ ID NO: 4, or SEQ ID NO: 5;
(e) encoding a polypeptide as set forth in any of SEQ ID NO: 2, SEQ ID NO: 4, or SEQ ID NO: 5 with at least one modification that is an amino acid substitution, amino acid insertion, amino acid deletion, C-terminal truncation, or N-terminal truncation, wherein the encoded polypeptide has an activity of the polypeptide set forth in any of SEQ ID NO: 2, SEQ ID NO: 4, or SEQ ID NO: 5;
(f) of any of (a)-(e) comprising a fragment of at least about 16 nucleotides;
(g) that hybridizes under at least moderately stringent conditions to the complement of the nucleotide sequence of any of (a)-(f); wherein the nucleic acid molecule encodes a polypeptide having an activity of the polypeptide set forth in any of SEQ ID NO: 2, SEQ ID NO: 4, or SEQ ID NO: 5; or
(h) complementary to the nucleotide sequence of any of (a)-(g).

The present invention also provides a method for treating, preventing, or ameliorating a cartilage-related disease, condition, or disorder comprising administering to a patient an isolated nucleic acid molecule comprising a nucleotide sequence:
(a) as set forth in either SEQ ID NO: 1 or SEQ ID NO: 3;
(b) encoding the polypeptide as set forth in any of SEQ ID NO: 2, SEQ ID NO: 4, or SEQ ID NO: 5;
(c) that hybridizes under at least moderately stringent conditions to the complement of the nucleotide sequence of either (a) or (b), wherein the nucleic acid molecule encodes polypeptide having an activity of the polypeptide set forth in any of SEQ ID NO: 2, SEQ ID NO: 4, or SEQ ID NO: 5; or
(d) complementary to the nucleotide sequence of any of (a)-(c).

The present invention also provides a method for treating, preventing, or ameliorating a cartilage-related disease, condition, or disorder comprising administering to a patient an isolated nucleic acid molecule comprising:
(a) a nucleotide sequence encoding a polypeptide that is at least about 70 percent identical to the polypeptide as set forth in any of SEQ ID NO: 2, SEQ ID NO: 4, or SEQ ID NO: 5, wherein the encoded polypeptide has an activity of the polypeptide set forth in any of SEQ ID NO: 2, SEQ ID NO: 4, or SEQ ID NO: 5;
(b) a nucleotide sequence encoding an allelic variant or splice variant of the nucleotide sequence as set forth in either SEQ ID NO: 1 or SEQ ID NO: 3, or the nucleotide sequence of (a);
(c) a region of the nucleotide sequence of any of SEQ ID NO: 1 or SEQ ID NO: 3, or the nucleotide sequence of (a) or (b) encoding a polypeptide fragment of at least about 25 amino acid residues, wherein the polypeptide fragment has an activity of the encoded polypeptide set forth in any of SEQ ID NO: 2, SEQ ID NO: 4, or SEQ ID NO: 5, or is antigenic;
(d) a region of the nucleotide sequence of any of SEQ ID NO: 1 or SEQ ID NO: 3, or the nucleotide sequence of any of (a)-(c) comprising a fragment of at least about 16 nucleotides;
(e) a nucleotide sequence that hybridizes under at least moderately stringent conditions to the complement of the nucleotide sequence of any of (a)-(d), wherein the nucleic acid molecule encodes a polypeptide having an activity of the polypeptide set forth in any of SEQ ID NO: 2, SEQ ID NO: 4, or SEQ ID NO: 5; or
(f) a nucleotide sequence complementary to the nucleotide sequence of any of (a)-(e).

The present invention also provides a method for treating, preventing, or ameliorating a cartilage-related disease, condition, or disorder comprising administering to a patient an isolated nucleic acid molecule comprising:
(a) encoding a polypeptide as set forth in any of SEQ ID NO: 2, SEQ ID NO: 4, or SEQ ID NO: 5 with at least one conservative amino acid substitution, wherein the encoded polypeptide has an activity of the polypeptide set forth in any of SEQ ID NO: 2, SEQ ID NO: 4, or SEQ ID NO: 5;
(b) encoding a polypeptide as set forth in any of SEQ ID NO: 2, SEQ ID NO: 4, or SEQ ID NO: 5 with at least one amino acid insertion, wherein the encoded polypeptide has an activity of the polypeptide set forth in any of SEQ ID NO: 2, SEQ ID NO: 4, or SEQ ID NO: 5;
(c) encoding a polypeptide as set forth in any of SEQ ID NO: 2, SEQ ID NO: 4, or SEQ ID NO: 5 with at least one amino acid deletion, wherein the encoded polypeptide has an activity of the polypeptide set forth in any of SEQ ID NO: 2, SEQ ID NO: 4, or SEQ ID NO: 5;
(d) encoding a polypeptide as set forth in any of SEQ ID NO: 2, SEQ ID NO: 4, or SEQ ID NO: 5 that has a C- and/or N- terminal truncation, wherein the encoded polypeptide has an activity of the polypeptide set forth in any of SEQ ID NO: 2, SEQ ID NO: 4, or SEQ ID NO: 5;
(e) encoding a polypeptide as set forth in any of SEQ ID NO: 2, SEQ ID NO: 4, or SEQ ID NO: 5 with at least one modification that is an amino acid substitution, amino acid insertion, amino acid deletion, C-terminal truncation, or N-terminal truncation, wherein the encoded polypeptide has an activity of the polypeptide set forth in any of SEQ ID NO: 2, SEQ ID NO: 4, or SEQ ID NO: 5;
(f) of any of (a)-(e) comprising a fragment of at least about 16 nucleotides;
(g) that hybridizes under at least moderately stringent conditions to the complement of the nucleotide sequence of any of (a)-(f); wherein the nucleic acid molecule encodes a polypeptide having an activity of the polypeptide set forth in any of SEQ ID NO: 2, SEQ ID NO: 4, or SEQ ID NO: 5; or
(h) complementary to the nucleotide sequence of any of (a)-(g).

The present invention also provides a method for treating, preventing, or ameliorating a bone-related disease, condition, or disorder comprising administering to a patient an isolated polypeptide comprising an amino acid sequence as set forth in any of SEQ ID NO: 2, SEQ ID NO: 4, or SEQ ID NO: 5.

The present invention also provides a method for treating, preventing, or ameliorating a bone-related disease, condition, or disorder comprising administering to a patient an isolated polypeptide comprising:
(a) an amino acid sequence for an ortholog of any of SEQ ID NO: 2, SEQ ID NO: 4, or SEQ ID NO: 5;
(b) an amino acid sequence that is at least about 70 percent identical to the amino acid sequence of any of SEQ ID NO: 2, SEQ ID NO: 4, or SEQ ID NO: 5, wherein the polypeptide has an activity of the polypeptide set forth any of SEQ ID NO: 2, SEQ ID NO: 4, or SEQ ID NO: 5;
(c) a fragment of the amino acid sequence set forth in any of SEQ ID NO: 2, SEQ ID NO: 4, or SEQ ID NO: 5 comprising at least about 25 amino acid residues, wherein the fragment has an activity of the polypeptide set forth in any of SEQ ID NO: 2, SEQ ID NO: 4, or SEQ ID NO: 5, or is antigenic; or
(d) an amino acid sequence for an allelic variant or splice variant of the amino acid sequence as set forth in any of SEQ ID NO: 2, SEQ ID NO: 4, or SEQ ID NO: 5, or the amino acid sequence of either (a) or (b).

The present invention also provides a method for treating, preventing, or ameliorating a bone-related disease, condition, or disorder comprising administering to a patient an isolated polypeptide comprising an amino acid sequence as set forth in SEQ ID NO: 2, SEQ ID NO: 4, or SEQ ID NO: 5:
(a) with at least one conservative amino acid substitution;
(b) with at least one amino acid insertion;
(c) with at least one amino acid deletion;
(d) the amino acid sequence as set forth in any of SEQ ID NO: 2, SEQ ID NO: 4, or SEQ ID NO: 5 that has a C- and/or N- terminal truncation; or
(e) with at least one modification that is an amino acid substitution, amino acid insertion, amino acid deletion, C-terminal truncation, or N-terminal truncation;
wherein the polypeptide has an activity of the polypeptide set forth in any of SEQ ID NO: 2, SEQ ID NO: 4, or SEQ ID NO: 5.

The present invention also provides a method for treating, preventing, or ameliorating a cartilage-related disease, condition, or disorder comprising administering to a patient an isolated polypeptide comprising an amino acid sequence as set forth in any of SEQ ID NO: 2, SEQ ID NO: 4, or SEQ ID NO: 5.

The present invention also provides a method for treating, preventing, or ameliorating a cartilage-related disease, condition, or disorder comprising administering to a patient an isolated polypeptide comprising:
(a) an amino acid sequence for an ortholog of any of SEQ ID NO: 2, SEQ ID NO: 4, or SEQ ID NO: 5;
(b) an amino acid sequence that is at least about 70 percent identical to the amino acid sequence of any of SEQ ID NO: 2, SEQ ID NO: 4, or SEQ ID NO: 5, wherein the polypeptide has an activity of the polypeptide set forth any of SEQ ID NO: 2, SEQ ID NO: 4, or SEQ ID NO: 5;
(c) a fragment of the amino acid sequence set forth in any of SEQ ID NO: 2, SEQ ID NO: 4, or SEQ ID NO: 5 comprising at least about 25 amino acid residues, wherein the fragment has an activity of the polypeptide set forth in any of SEQ ID NO: 2, SEQ ID NO: 4, or SEQ ID NO: 5, or is antigenic; or
(d) an amino acid sequence for an allelic variant or splice variant of the amino acid sequence as set forth in any of SEQ ID NO: 2, SEQ ID NO: 4, or SEQ ID NO: 5, or the amino acid sequence of either (a) or (b).

The present invention also provides a method for treating, preventing, or ameliorating a cartilage-related disease, condition, or disorder comprising administering to a patient an isolated polypeptide comprising an amino acid sequence as set forth in SEQ ID NO: 2, SEQ ID NO: 4, or SEQ ID NO: 5:
(a) with at least one conservative amino acid substitution;
(b) with at least one amino acid insertion;
(c) with at least one amino acid deletion;
(d) the amino acid sequence as set forth in any of SEQ ID NO: 2, SEQ ID NO: 4, or SEQ ID NO: 5 that has a C- and/or N- terminal truncation; or
(e) with at least one modification that is an amino acid substitution, amino acid insertion, amino acid deletion, C-terminal truncation, or N-terminal truncation;
wherein the polypeptide has an activity of the polypeptide set forth in any of SEQ ID NO: 2, SEQ ID NO: 4, or SEQ ID NO: 5.

The present invention provides a transgenic non-human mammal that is a HEH4 knock-out. The present invention also provides a process for obtaining an anti-HEH4 antibody comprising immunizing a transgenic non-human mammal that is a HEH4 knock-out with an amino acid sequence as set forth in any of SEQ ID NO: 2, SEQ ID NO: 4, or SEQ ID NO: 5 or a fragment thereof.

The present invention also provides a method for modulating by increasing bone mineral density or bone strength in an individual comprising administering to the individual a selective binding agent or fragment thereof that specifically binds a polypeptide comprising an amino acid sequence as set forth in any of SEQ ID NO: 2, SEQ ID NO: 4, or SEQ ID NO: 5.

The present invention also provides a method for modulating by increasing bone mineral density or bone strength in an individual comprising administering to the individual a selective binding agent or fragment thereof that specifically binds a polypeptide comprising:
(a) an amino acid sequence for an ortholog of any of SEQ ID NO: 2, SEQ ID NO: 4, or SEQ ID NO: 5;
(b) an amino acid sequence that is at least about 70 percent identical to the amino acid sequence of any of SEQ ID NO: 2, SEQ ID NO: 4, or SEQ ID NO: 5, wherein the polypeptide has an activity of the polypeptide set forth any of SEQ ID NO: 2, SEQ ID NO: 4, or SEQ ID NO: 5;
(c) a fragment of the amino acid sequence set forth in any of SEQ ID NO: 2, SEQ ID NO: 4, or SEQ ID NO: 5 comprising at least about 25 amino acid residues, wherein the fragment has an activity of the polypeptide set forth in any of SEQ ID NO: 2, SEQ ID NO: 4, or SEQ ID NO: 5, or is antigenic; or
(d) an amino acid sequence for an allelic variant or splice variant of the amino acid sequence as set forth in any of SEQ ID NO: 2, SEQ ID NO: 4, or SEQ ID NO: 5, or the amino acid sequence of either (a) or (b).

The present invention also provides a method for modulating by increasing bone mineral density or bone strength in an individual comprising administering to the individual a selective binding agent or fragment thereof that specifically binds a polypeptide comprising an amino acid sequence as set forth in SEQ ID NO: 2, SEQ ID NO: 4, or SEQ ID NO: 5:
(a) with at least one conservative amino acid substitution;
(b) with at least one amino acid insertion;
(c) with at least one amino acid deletion;
(d) the amino acid sequence as set forth in any of SEQ ID NO: 2, SEQ ID NO: 4, or SEQ ID NO: 5 that has a C- and/or N- terminal truncation; or
(e) with at least one modification that is an amino acid substitution, amino acid insertion, amino acid deletion, C-terminal truncation, or N-terminal truncation;
wherein the polypeptide has an activity of the polypeptide set forth in any of SEQ ID NO: 2, SEQ ID NO: 4, or SEQ ID NO: 5.

The present invention also provides a method for modulating by increasing bone mineral density or bone strength in an individual comprising administering to the individual an antagonist of a polypeptide comprising an amino acid sequence as set forth in any of SEQ ID NO: 2, SEQ ID NO: 4, or SEQ ID NO: 5.

The present invention also provides a method for modulating by increasing bone mineral density or bone strength in an individual comprising administering to the individual an antagonist of a polypeptide comprising:
(a) an amino acid sequence for an ortholog of any of SEQ ID NO: 2, SEQ ID NO: 4, or SEQ ID NO: 5;
(b) an amino acid sequence that is at least about 70 percent identical to the amino acid sequence of any of SEQ ID NO: 2, SEQ ID NO: 4, or SEQ ID NO: 5, wherein the polypeptide has an activity of the polypeptide set forth any of SEQ ID NO: 2, SEQ ID NO: 4, or SEQ ID NO: 5;
(c) a fragment of the amino acid sequence set forth in any of SEQ ID NO: 2, SEQ ID NO: 4, or SEQ ID NO: 5 comprising at least about 25 amino acid residues, wherein the fragment has an activity of the polypeptide set forth in any of SEQ ID NO: 2, SEQ ID NO: 4, or SEQ ID NO: 5, or is antigenic; or
(d) an amino acid sequence for an allelic variant or splice variant of the amino acid sequence as set forth in any of SEQ ID NO: 2, SEQ ID NO: 4, or SEQ ID NO: 5, or the amino acid sequence of either (a) or (b).

The present invention also provides a method for modulating by increasing bone mineral density or bone strength in an individual comprising administering to the individual an antagonist of a polypeptide comprising an amino acid sequence as set forth in SEQ ID NO: 2, SEQ ID NO: 4, or SEQ ID NO: 5:
(a) with at least one conservative amino acid substitution;
(b) with at least one amino acid insertion;
(c) with at least one amino acid deletion;
(d) the amino acid sequence as set forth in any of SEQ ID NO: 2, SEQ ID NO: 4, or SEQ ID NO: 5 that has a C- and/or N- terminal truncation; or
(e) with at least one modification that is an amino acid substitution, amino acid insertion, amino acid deletion, C-terminal truncation, or N-terminal truncation;
wherein the polypeptide has an activity of the polypeptide set forth in any of SEQ ID NO: 2, SEQ ID NO: 4, or SEQ ID NO: 5.

The present invention also provides a method for modulating by decreasing bone mineral density or bone strength in an individual comprising administering to the individual an agonist of a polypeptide comprising an amino acid sequence as set forth in any of SEQ ID NO: 2, SEQ ID NO: 4, or SEQ ID NO: 5.

The present invention also provides a method for modulating by decreasing bone mineral density or bone strength in an individual comprising administering to the individual an agonist of a polypeptide comprising:
(a) an amino acid sequence for an ortholog of any of SEQ ID NO: 2, SEQ ID NO: 4, or SEQ ID NO: 5;
(b) an amino acid sequence that is at least about 70 percent identical to the amino acid sequence of any of SEQ ID NO: 2, SEQ ID NO: 4, or SEQ ID NO: 5, wherein the polypeptide has an activity of the polypeptide set forth any of SEQ ID NO: 2, SEQ ID NO: 4, or SEQ ID NO: 5;
(c) a fragment of the amino acid sequence set forth in any of SEQ ID NO: 2, SEQ ID NO: 4, or SEQ ID NO: 5 comprising at least about 25 amino acid residues, wherein the fragment has an activity of the polypeptide set forth in any of SEQ ID NO: 2, SEQ ID NO: 4, or SEQ ID NO: 5, or is antigenic; or
(d) an amino acid sequence for an allelic variant or splice variant of the amino acid sequence as set forth in any of SEQ ID NO: 2, SEQ ID NO: 4, or SEQ ID NO: 5, or the amino acid sequence of either (a) or (b).

The present invention also provides a method for modulating by decreasing bone mineral density or bone strength in an individual comprising administering to the individual an agonist of a polypeptide comprising an amino acid sequence as set forth in SEQ ID NO: 2, SEQ ID NO: 4, or SEQ ID NO: 5:
(a) with at least one conservative amino acid substitution;
(b) with at least one amino acid insertion;
(c) with at least one amino acid deletion;
(d) the amino acid sequence as set forth in any of SEQ ID NO: 2, SEQ ID NO: 4, or SEQ ID NO: 5 that has a C- and/or N- terminal truncation; or
(e) with at least one modification that is an amino acid substitution, amino acid insertion, amino acid deletion, C-terminal truncation, or N-terminal truncation;
wherein the polypeptide has an activity of the polypeptide set forth in any of SEQ ID NO: 2, SEQ ID NO: 4, or SEQ ID NO: 5.

The present invention also provides a method for modulating by decreasing bone mineral density or bone strength in an individual comprising administering to the individual an isolated nucleic acid molecule comprising a nucleotide sequence:
(a) as set forth in either SEQ ID NO: 1 or SEQ ID NO: 3;
(b) encoding the polypeptide as set forth in any of SEQ ID NO: 2, SEQ ID NO: 4, or SEQ ID NO: 5;
(c) that hybridizes under at least moderately stringent conditions to the complement of the nucleotide sequence of either (a) or (b), wherein the nucleic acid molecule encodes polypeptide having an activity of the polypeptide set forth in any of SEQ ID NO: 2, SEQ ID NO: 4, or SEQ ID NO: 5; or
(d) complementary to the nucleotide sequence of any of (a)-(c).

The present invention also provides a method for modulating by decreasing bone mineral density or bone strength in an individual comprising administering to the individual an isolated nucleic acid molecule comprising:
(a) a nucleotide sequence encoding a polypeptide that is at least about 70 percent identical to the polypeptide as set forth in any of SEQ ID NO: 2, SEQ ID NO: 4, or SEQ ID NO: 5, wherein the encoded polypeptide has an activity of the polypeptide set forth in any of SEQ ID NO: 2, SEQ ID NO: 4, or SEQ ID NO: 5;
(b) a nucleotide sequence encoding an allelic variant or splice variant of the nucleotide sequence as set forth in either SEQ ID NO: 1 or SEQ ID NO: 3, or the nucleotide sequence of (a);
(c) a nucleotide sequence that hybridizes under at least moderately stringent conditions to the complement of the nucleotide sequence of either (a) or (b), wherein the nucleic acid molecule encodes a polypeptide having an activity of the polypeptide set forth in any of SEQ ID NO: 2, SEQ ID NO: 4, or SEQ ID NO: 5.

The present invention also provides a method for modulating by decreasing bone mineral density or bone strength in an individual comprising administering to the individual an isolated nucleic acid molecule comprising:
(a) encoding a polypeptide as set forth in any of SEQ ID NO: 2, SEQ ID NO: 4, or SEQ ID NO: 5 with at least one conservative amino acid substitution, wherein the encoded polypeptide has an activity of the polypeptide set forth in any of SEQ ID NO: 2, SEQ ID NO: 4, or SEQ ID NO: 5; or
(b) that hybridizes under at least moderately stringent conditions to the complement of the nucleotide sequence of (a), wherein the nucleic acid molecule encodes a polypeptide having an activity of the polypeptide set forth in any of SEQ ID NO: 2, SEQ ID NO: 4, or SEQ ID NO: 5.

The present invention also provides a method for modulating by increasing bone mineral density or bone strength in an individual comprising administering to the individual an isolated nucleic acid molecule comprising:
(a) a region of the nucleotide sequence of:
   (i) either SEQ ID NO: 1 or SEQ ID NO: 3;
   (ii) encoding a polypeptide that is at least about 70 percent identical to the polypeptide as set forth in any of SEQ ID NO: 2, SEQ ID NO: 4, or SEQ ID NO: 5, wherein the encoded polypeptide has an activity of the polypeptide set forth in any of SEQ ID NO: 2, SEQ ID NO: 4, or SEQ ID NO: 5; or
   (iii) encoding an allelic variant or splice variant of the nucleotide sequence as set forth in either SEQ ID NO: 1 or SEQ ID NO: 3, or the nucleotide sequence of (ii);
   wherein the region of the nucleotide sequence encodes a polypeptide fragment of at least about 25 amino acid residues;
(b) a region of the nucleotide sequence of:
   (i) either SEQ ID NO: 1 or SEQ ID NO: 3;
   encoding a polypeptide that is at least about 70 percent identical to the polypeptide as set forth in any of SEQ ID NO: 2, SEQ ID NO: 4, or SEQ ID NO: 5, wherein the encoded polypeptide has an activity of the polypeptide set forth in any of SEQ ID NO: 2, SEQ ID NO: 4, or SEQ ID NO: 5; or
   (iii) encoding an allelic variant or splice variant of the nucleotide sequence as set forth in either SEQ ID NO: 1 or SEQ ID NO: 3, or the nucleotide sequence of (ii);
   wherein the nucleotide sequence comprises a fragment of at least about 16 nucleotides;
(c) a nucleotide sequence complementary to the nucleotide sequence of either (a) or (b).

The present invention also provides a method for modulating by increasing bone mineral density or bone strength in an individual comprising administering to the individual an isolated nucleic acid molecule comprising:
(a) encoding a polypeptide as set forth in any of SEQ ID NO: 2, SEQ ID NO: 4, or SEQ ID NO: 5 with at least one amino acid insertion, wherein the encoded polypeptide has an activity of the polypeptide set forth in any of SEQ ID NO: 2, SEQ ID NO: 4, or SEQ ID NO: 5;
(b) encoding a polypeptide as set forth in any of SEQ ID NO: 2, SEQ ID NO: 4, or SEQ ID NO: 5 with at least one amino acid deletion, wherein the encoded polypeptide has an activity of the polypeptide set forth in any of SEQ ID NO: 2, SEQ ID NO: 4, or SEQ ID NO: 5;
(c) encoding a polypeptide as set forth in any of SEQ ID NO: 2, SEQ ID NO: 4, or SEQ ID NO: 5 that has a C- and/or N- terminal truncation, wherein the encoded polypeptide has an activity of the polypeptide set forth in any of SEQ ID NO: 2, SEQ ID NO: 4, or SEQ ID NO: 5;
(d) encoding a polypeptide as set forth in any of SEQ ID NO: 2, SEQ ID NO: 4, or SEQ ID NO: 5 with at least one modification that is an amino acid insertion, amino acid deletion, C-terminal truncation, or N-terminal truncation, wherein the encoded polypeptide has an activity of the polypeptide set forth in any of SEQ ID NO: 2, SEQ ID NO: 4, or SEQ ID NO: 5;
(e) of any of (a)-(d) comprising a fragment of at least about 16 nucleotides;
(f) that hybridizes under at least moderately stringent conditions to the complement of the nucleotide sequence of any of (a)-(e); wherein the nucleic acid molecule encodes a polypeptide having an activity of the polypeptide set forth in any of SEQ ID NO: 2, SEQ ID NO: 4, or SEQ ID NO: 5; or
(g) complementary to the nucleotide sequence of any of (a)-(f).

The present invention also provides a method for modulating by decreasing bone mineral density or bone strength in an individual comprising administering to the individual an isolated polypeptide comprising an amino acid sequence as set forth in any of SEQ ID NO: 2, SEQ ID NO: 4, or SEQ ID NO: 5.

The present invention also provides a method for modulating by decreasing bone mineral density or bone strength in an individual comprising administering to the individual an isolated polypeptide comprising:
(a) an amino acid sequence for an ortholog of any of SEQ ID NO: 2, SEQ ID NO: 4, or SEQ ID NO: 5;
(b) an amino acid sequence that is at least about 70 percent identical to the amino acid sequence of any of SEQ ID NO: 2, SEQ ID NO: 4, or SEQ ID NO: 5, wherein the polypeptide has an activity of the polypeptide set forth any of SEQ ID NO: 2, SEQ ID NO: 4, or SEQ ID NO: 5; or
(c) an amino acid sequence for an allelic variant or splice variant of the amino acid sequence as set forth in any of SEQ ID NO: 2, SEQ ID NO: 4, or SEQ ID NO: 5, or the amino acid sequence of either (a) or (b).

The present invention also provides a method for modulating by decreasing bone mineral density or bone strength in an individual comprising administering to the individual an isolated polypeptide comprising an amino acid sequence as set forth in SEQ ID NO: 2, SEQ ID NO: 4, or SEQ ID NO: 5 with at least one conservative amino acid substitution, wherein the polypeptide has an activity of the polypeptide set forth in any of SEQ ID NO: 2, SEQ ID NO: 4, or SEQ ID NO: 5.

The present invention also provides a method for modulating by increasing bone mineral density or bone strength in an individual comprising administering to the individual an isolated polypeptide comprising a fragment of the amino acid sequence:
(a) for an ortholog of any of SEQ ID NO: 2, SEQ ID NO: 4, or SEQ ID NO: 5;
(b) that is at least about 70 percent identical to the amino acid sequence of any of SEQ ID NO: 2, SEQ ID NO: 4, or SEQ ID NO: 5;
(c) set forth in any of SEQ ID NO: 2, SEQ ID NO: 4, or SEQ ID NO: 5 comprising at least about 25 amino acid residues; or
(d) for an allelic variant or splice variant of the amino acid sequence as set forth in any of SEQ ID NO: 2, SEQ ID NO: 4, or SEQ ID NO: 5, or the amino acid sequence of either (a) or (b).

The present invention also provides a method for modulating by increasing bone mineral density or bone strength in an individual comprising administering to the individual an isolated polypeptide comprising an amino acid sequence as set forth in SEQ ID NO: 2, SEQ ID NO: 4, or SEQ ID NO: 5:
(a) with at least one amino acid insertion;
(b) with at least one amino acid deletion;
(c) the amino acid sequence as set forth in any of SEQ ID NO: 2, SEQ ID NO: 4, or SEQ ID NO: 5 that has a C- and/or N- terminal truncation; or
(d) with at least one modification that is an amino acid insertion, amino acid deletion, C-terminal truncation, or N-terminal truncation.

The present invention provides a method of assaying test molecules to identify a test molecule that binds to a HEH4 polypeptide. The method comprises contacting a HEH4 polypeptide with a test molecule to determine the extent of binding of the test molecule to the polypeptide. The method further comprises determining whether such test molecules are agonists or antagonists of a HEH4 polypeptide. The present invention further provides a method of testing the impact of molecules on the expression of HEH4 polypeptide or on the activity of HEH4 polypeptide.

Methods of regulating expression and modulating (i.e., increasing or decreasing) levels of a HEH4 polypeptide are also encompassed by the invention. One method comprises administering to an animal a nucleic acid molecule encoding a HEH4 polypeptide. In another method, a nucleic acid molecule comprising elements that regulate or modulate the expression of a HEH4 polypeptide may be administered. Examples of these methods include gene therapy, cell therapy, and anti-sense therapy as further described herein.

In another aspect of the present invention, the HEH4 polypeptides may be used for identifying receptors thereof (“HEH4 polypeptide receptors”). Various forms of “expression cloning” have been extensively used to clone receptors for protein ligands. See, e.g., Simonsen and Lodish, 1994, Trends Pharmacol. Sci. 15:437-41 and Tartaglia et al., 1995, Cell 83:1263-71. The isolation of a HEH4 polypeptide receptor is useful for identifying or developing novel agonists and antagonists of the HEH4 polypeptide signaling pathway. Such agonists and antagonists include soluble HEH4 polypeptide receptors, anti-HEH4 polypeptide receptor-selective binding agents (such as antibodies and derivatives thereof), small molecules, and antisense oligonucleotides, any of which can be used for treating one or more disease or disorder, including those disclosed herein.

HEH4 polypeptides may also be useful for identifying ligands thereof. Various forms of “expression cloning” have been used for cloning ligands for receptors (See, e.g., Davis et al., 1996, Cell, 87:1161-69). These and other HEH4 ligand cloning experiments are described in greater detail herein. Isolation of the HEH4 ligand(s) allows for the identification or development of novel agonists or antagonists of the HEH4 signaling pathway. Such agonists and antagonists include HEH4 ligand(s), anti-HEH4 ligand antibodies and derivatives thereof, small molecules, or antisense oligonucleotides, any of which can be used for potentially treating one or more diseases or disorders, including those recited herein.

### BRIEF DESCRIPTION OF THE FIGURES

FIGS. 1A-1C illustrate the nucleotide sequence of the human HEH4 gene (SEQ ID NO: 1) and the deduced amino acid sequence of human HEH4 (SEQ ID NO: 2);

FIGS. 2A-2C illustrate the nucleotide sequence of the rat HEH4 gene (SEQ ID NO: 3) and the deduced amino acid sequence of rat HEH4 (SEQ ID NO: 4);

FIG. 3 illustrates an alignment of the amino acid sequences for human HEH4 (SEQ ID NO: 2) and murine HEH4 (SEQ ID NO: 5); the deduced murine and predicted human HEH4 signal sequences (bold underline), predicted Ig regions for both proteins (underline), and predicted transmembrane domains for both proteins (double underline) are indicated;

FIG. 4 illustrates the expression of HEH4 mRNA as detected by Northern blot analysis of crushed bone samples from normal adult mouse, OPG transgenic mouse, OPG knockout mouse, and an osteoblast lineage cell line (MC3T3-E1 cells);

FIG. 5 illustrates the expression of HEH4 mRNA as detected by in situ hybridization in normal adult mouse lung, liver, intestine, ovary, and uterine tissue;

FIG. 6 illustrates the expression of HEH4 mRNA as detected by in situ hybridization in normal adult mouse bone (femur) and cartilage (trachea). In normal adult mouse bone, HEH4 signal was detected over osteoblast-type cells (second row), but little to no HEH4 signal was detected over osteoclasts (indicated by arrows). In normal adult mouse trachea, HEH4 signal was detected primarily over perichondral cells, with a much lower density of grains detected over chondrocytes;

FIG. 7 illustrates the expression of HEH4 mRNA as detected by in situ hybridization in normal adult mouse brain, heart, kidney, and spleen tissue;

FIG. 8 illustrates the expression of HEH4 mRNA as detected by in situ hybridization in bone tissue from normal adult mouse, OPG transgenic mouse, OPG knockout mouse, and OPGL-treated mouse. A slightly increased HEH4 signal was detected in OPG knockout and OPGL-treated versus normal and OPG transgenic mice;

FIGS. 9A-9B illustrate the expression of HEH4 mRNA as detected by in situ hybridization in (A) knee tissue from humans with degenerative joint disease (DJD) or rheumatoid arthritis (RA) or (B) knee tissue from humans with osteoarthritis/degenerative joint disease (OA/DJD). A strong HEH4 signal was detected in osteoblasts and a low HEH4 signal was detected in chrondrocytes from both DJD and RA patients, a low to moderate HEH4 signal was detected in osteoblasts from the OA/DJD patient, and virtually no signal was detected in bone from normal individuals (H&E=hematoxylin and eosin; ISH=in situ hybridization);

FIGS. 10A-10B illustrate the results of peripheral quantitative computed tomography (pQCT) analysis; measurements from the proximal tibia (A) show a statistically significant decrease in trabecular bone mineral density (BMD) in HEH4 transgenic animals compared to wildtype littermate animals; measurements from the lumbar vertebrae (B) of HEH4 transgenic mice show a statistically significant decrease in total and cortical BMD compared to wildtype littermates.

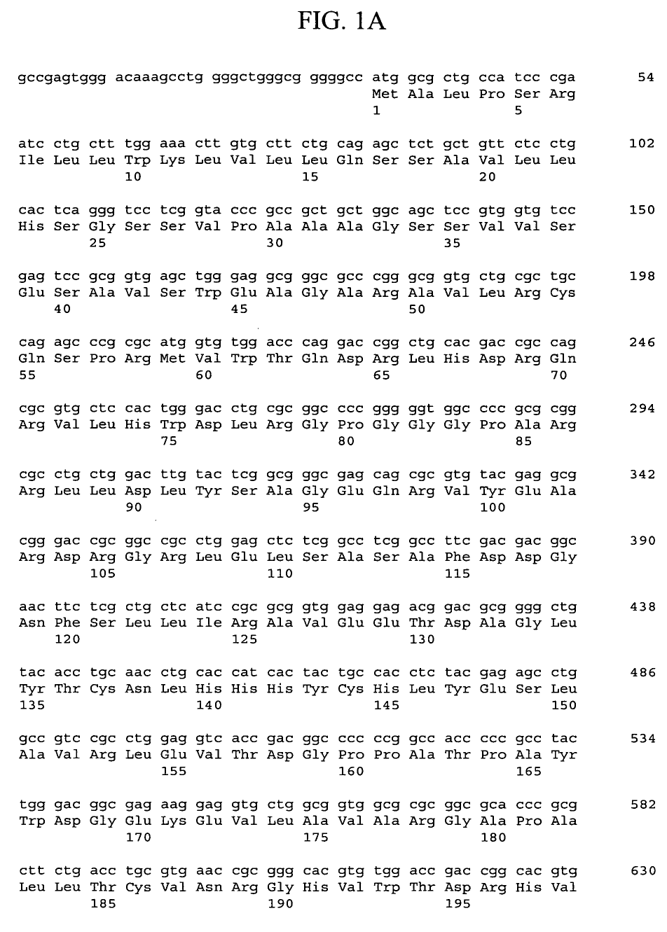

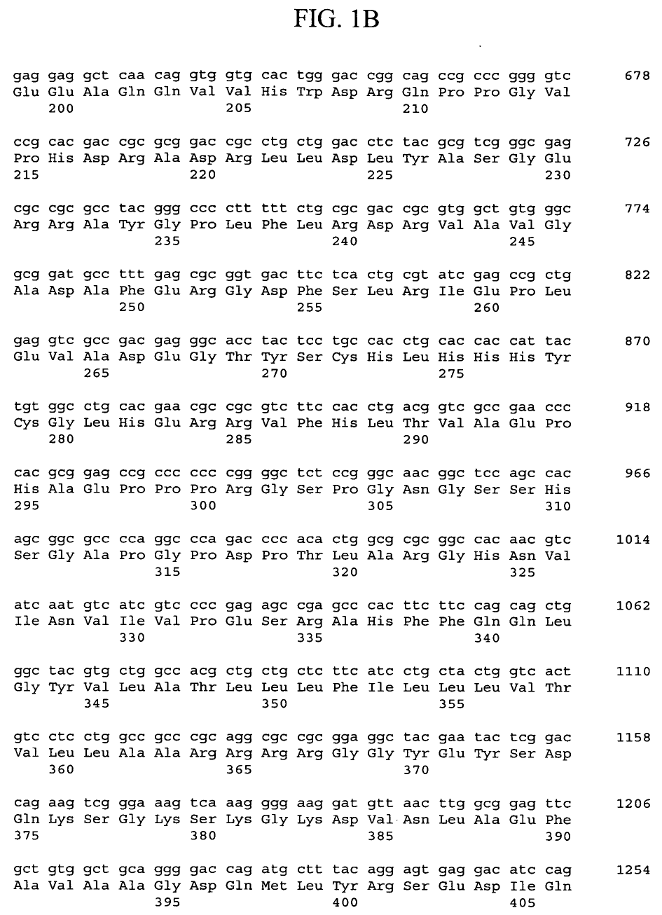

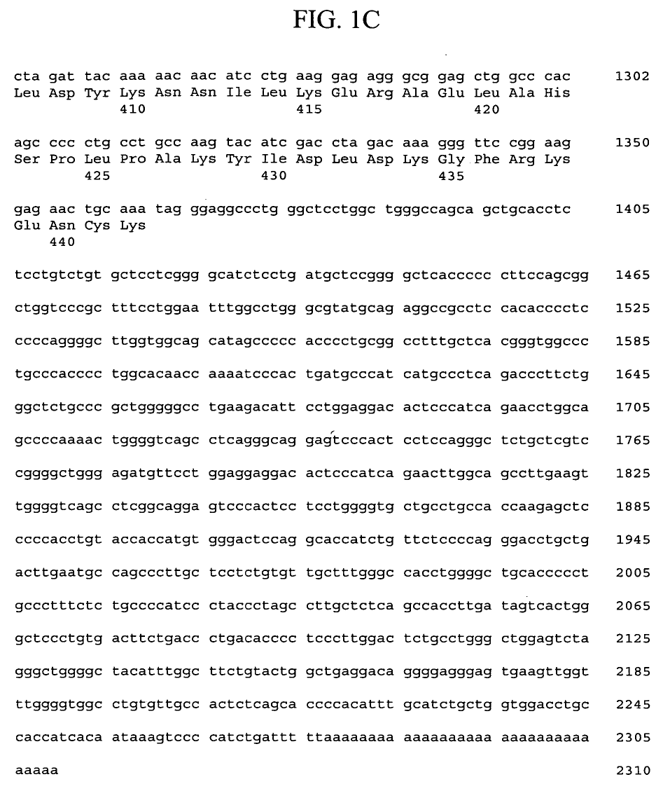

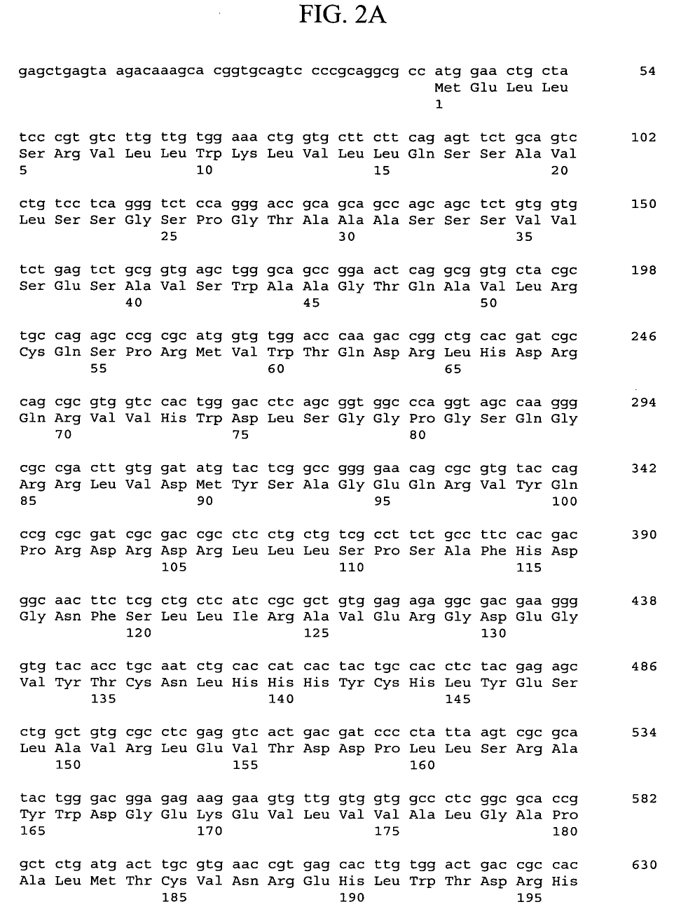

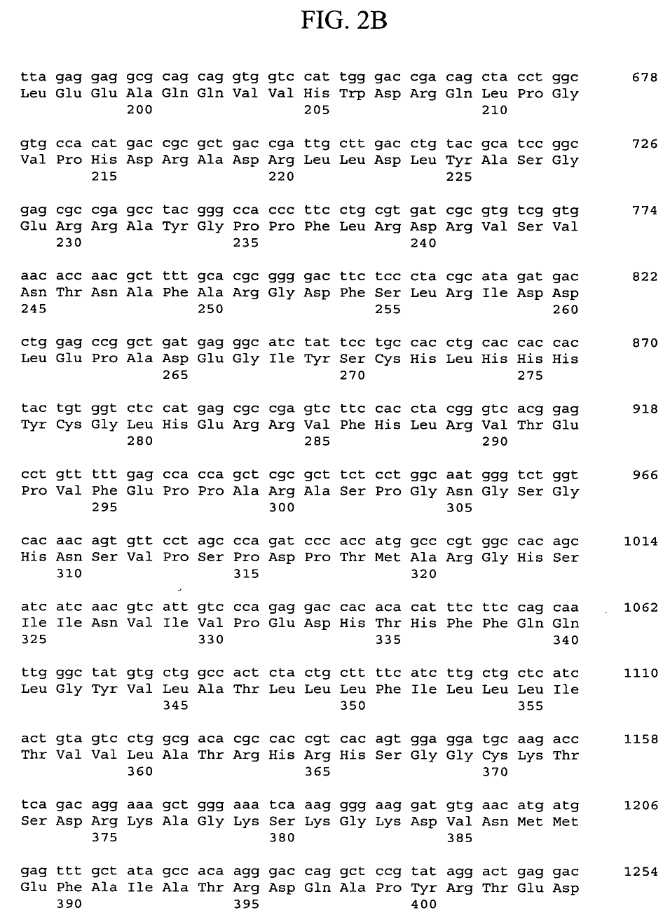

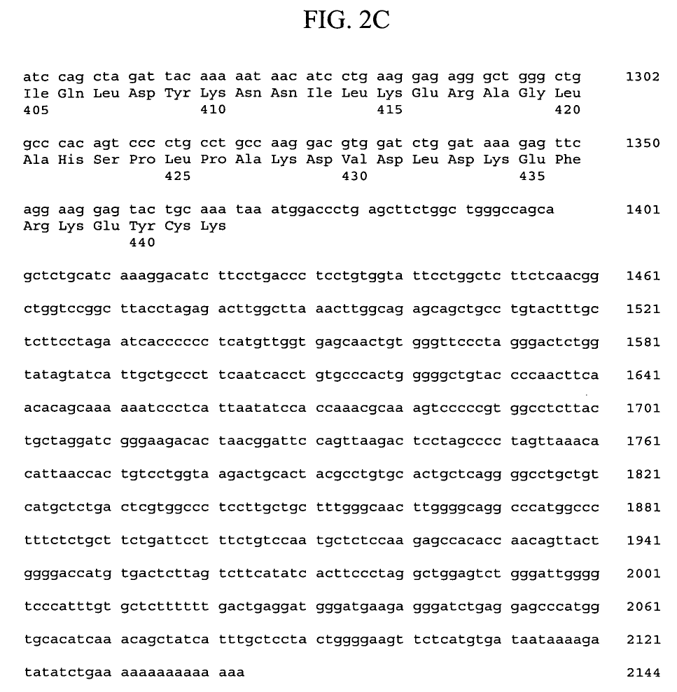

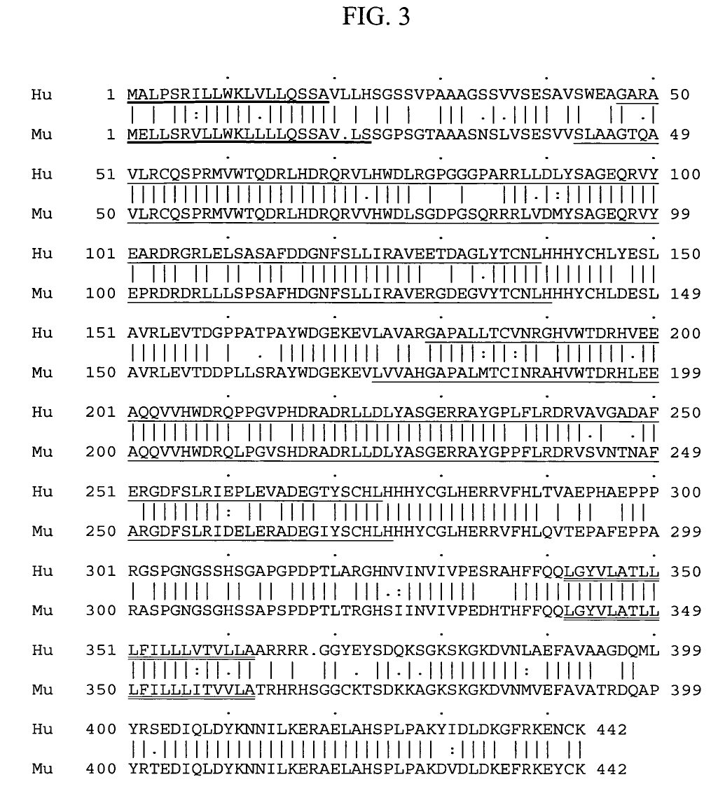

### DETAILED DESCRIPTION OF THE INVENTION

A series of microarray experiments was performed to identify genes differentially expressed in in vivo mouse models of bone disease. Expression profile analysis led to the identification of HEH4, a gene that encodes a 442 amino acid polypeptide with a predicted transmembrane domain and two extracellular Ig regions, but with little or no homology to any known gene family. Subsequent follow-up functionation experiments focused on generating data from “native environment”/in vivo samples and systems such as Northern blots of whole bone RNA, and in situ hybridization with samples from adult mice, elderly humans, and transgenic mice instead of relying solely upon data from fetal bones or cartilage, or cells of the osteoblast or chondrocyte lineage that were no longer in their “native” whole bone environment (e.g., isolated osteoblast or chondrocyte lineage cells, such cells grown in culture, established osteoblast or chondrocyte lineage cell lines).

To confirm that HEH4 plays an important role in adult skeletal (i.e., bone or cartilage) physiology and pathophysiology, Northern and in situ expression analyses were conducted. From in situ hybridization analysis of murine tissues, HEH4 expression was detected in a number of embryonic and adult tissues, with the highest expression being observed in the alveolar cells of the lungs, periosteal cells, perichondral cells, and connective tissue elements. HEH4 expression was also seen in osteoblasts, but not in osteoclasts, of adult mouse bone. Increased HEH4 expression was also noted in adult mouse bone models, including osteoprotegerin (OPG) knockout mouse bone and osteoprotegerin ligand (OPGL)-treated mouse bone. In situ analysis of a panel of knee samples from normal elderly humans as well as elderly humans afflicted with osteoarthritis or degenerative joint disease, rheumatoid arthritis, or osteoporosis showed that HEH4 was consistently expressed by cells of the osteoblast lineage. The level of HEH4 expression was higher in the osteoblasts and bone lining cells of the diseased as compared to normal tissue samples. Additionally, low HEH4 expression was detected in the chondrocytes of articular cartilage in several of the osteoarthritic, dejenerative joint disease, and rheumatoid arthritic samples.

The results of such expression analyses, coupled with the results of the original microarray strategy, suggest that HEH4 nucleic acid molecules, polypeptides, and antagonists and agonists thereof can be used to treat, diagnose, ameliorate, or prevent bone- and cartilage-related diseases such as osteoporosis, osteoarthritis or degenerative joint disease, and rheumatoid arthritis. Significant in this regard is the discovery of HEH4 expression in bone cells (osteoblast lineage) and chondrocytes in elderly humans, which for many bone and cartilage diseases, such as osteoporosis and osteoarthritis, is the target population for treatment. In other words, this persistent expression of HEH4 into the elderly years indicates that HEH4 is likely to play a role in bone or cartilage biology in elderly humans and not just during human development or early adulthood.

To further investigate the in vivo role of HEH4 produced from cells of the osteoblast lineage, transgenic mice were generated that overexpressed HEH4 from the rat Col1a1 (3.6-kb) promoter whose transgenic expression is largely restricted to cells of the osteoblast lineage. A transgenic phenotype was obtained thus demonstrating that modulation of HEH4 levels or activity in vivo can effect a biological change in a whole animal in vivo setting. More specifically, the highest transgenic expressors had low bone mineral density. In other words, increasing HEH4 levels or activity in bone in vivo results in a decrease in bone mineral density. As such it is very likely that one could cause an increase in bone mineral density or bone strength by decreasing HEH4 levels or activity in bone in vivo. Thus, molecules (for example, antagonists of HEH4) that can decrease human HEH4 levels or activity would be possible therapeutics for treating, ameliorating, or preventing diseases or disorders characterized by below normal bone mineral density or below normal bone strength, such as osteoporosis.

To provide support for this hypothesis, transgenic mice overexpressing a secreted form of HEH4 consisting of the extracellular region of the molecule under the control of the human ApoE promoter were analyzed. These mice showed an increase in trabecular bone compared to normal littermate controls. There was a positive correlation between RNA expression level and the increase in bone in the transgenic animals, suggesting that a soluble form of HEH4 functions as an antagonist of HEH4 function, and could be useful as a therapeutic agent to treat, ameliorate, or prevent diseases or disorders characterized by below normal bone mineral density, such as osteoporosis.

HEH4 was mapped to human chromosome 1p36.33 (hypothetical protein MGC3047), a locus linked to effects on bone mineral density (BMD). Devoto et al., have confirmed the existence of a candidate region conferring susceptibility to low BMD of the femoral neck on chromosome 1p36 (Devoto et al., 2001, Hum. Mol. Genet. 10:2447-52).

The section headings used herein are for organizational purposes only and are not to be construed as limiting the subject matter described. All references cited in this application are expressly incorporated by reference herein.

Definitions

The terms “HEH4 gene” or “HEH4 nucleic acid molecule” or “HEH4 10 polynucleotide” refer to a nucleic acid molecule comprising or consisting of a nucleotide sequence as set forth in either SEQ ID NO: 1 or SEQ ID NO: 3, a nucleotide sequence encoding the polypeptide as set forth in any of SEQ ID NO: 2, SEQ ID NO: 4, or SEQ ID NO: 5, and nucleic acid molecules as defined herein.

The term “HEH4 polypeptide allelic variant” refers to one of several possible naturally occurring alternate forms of a gene occupying a given locus on a chromosome of an organism or a population of organisms.

The term “HEH4 polypeptide splice variant” refers to a nucleic acid molecule, usually RNA, which is generated by alternative processing of intron sequences in an RNA transcript encoding a HEH4 polypeptide amino acid sequence as set forth in any of SEQ ID NO: 2, SEQ ID NO: 4, or SEQ ID NO: 5.

The term “isolated nucleic acid molecule” refers to a nucleic acid molecule of the invention that (1) has been separated from at least about 50 percent of proteins, lipids, carbohydrates, or other materials with which it is naturally found when total nucleic acid is isolated from the source cells, (2) is not linked to all or a portion of a polynucleotide to which the “isolated nucleic acid molecule” is linked in nature, (3) is operably linked to a polynucleotide which it is not linked to in nature, or (4) does not occur in nature as part of a larger polynucleotide sequence. Preferably, the isolated nucleic acid molecule of the present invention is substantially free from any other contaminating nucleic acid molecule(s) or other contaminants that are found in its natural environment that would interfere with its use in polypeptide production or its therapeutic, diagnostic, prophylactic or research use.

The term “nucleic acid sequence” or “nucleic acid molecule” refers to a DNA or RNA sequence. The term encompasses molecules formed from any of the known base analogs of DNA and RNA such as, but not limited to 4-acetylcytosine, 8-hydroxy-N6-methyladenosine, aziridinyl-cytosine, pseudoisocytosine, 5-(carboxyhydroxylmethyl) uracil, 5-fluorouracil, 5-bromouracil, 5-carboxymethylaminomethyl-2-thiouracil, 5-carboxy-methylaminomethyluracil, dihydrouracil, inosine, N6-iso-pentenyladenine, 1-methyladenine, 1-methylpseudouracil, 1-methylguanine, 1-methylinosine, 2,2-dimethyl-guanine, 2-methyladenine, 2-methylguanine, 3-methylcytosine, 5-methylcytosine, N6-methyladenine, 7-methylguanine, 5-methylaminomethyluracil, 5-methoxyamino-methyl-2-thiouracil, beta-D-mannosylqueosine, 5′ -methoxycarbonyl-methyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “vector” is used to refer to any molecule (e.g., nucleic acid, plasmid, or virus) used to transfer coding information to a host cell.

The term “expression vector” refers to a vector that is suitable for transformation of a host cell and contains nucleic acid sequences that direct and/or control the expression of inserted heterologous nucleic acid sequences. Expression includes, but is not limited to, processes such as transcription, translation, and RNA splicing, if introns are present.

The term “operably linked” is used herein to refer to an arrangement of flanking sequences wherein the flanking sequences so described are configured or assembled so as to perform their usual function. Thus, a flanking sequence operably linked to a coding sequence may be capable of effecting the replication, transcription and/or translation of the coding sequence. For example, a coding sequence is operably linked to a promoter when the promoter is capable of directing transcription of that coding sequence. A flanking sequence need not be contiguous with the coding sequence, so long as it functions correctly. Thus, for example, intervening untranslated yet transcribed sequences can be present between a promoter sequence and the coding sequence and the promoter sequence can still be considered “operably linked” to the coding sequence.

The term “host cell” is used to refer to a cell which has been transformed, or is capable of being transformed with a nucleic acid sequence and then of expressing a selected gene of interest. The term includes the progeny of the parent cell, whether or not the progeny is identical in morphology or in genetic make-up to the original parent, so long as the selected gene is present.

The term “HEH4 polypeptide” refers to a polypeptide comprising the amino acid sequence of any of SEQ ID NO: 2, SEQ ID NO: 4, or SEQ ID NO: 5 and related polypeptides. Related polypeptides include HEH4 polypeptide fragments, HEH4 polypeptide orthologs, HEH4 polypeptide variants, and HEH4 polypeptide derivatives, which possess at least one activity of the polypeptide set forth in any of SEQ ID NO: 2, SEQ ID NO: 4, or SEQ ID NO: 5. HEH4 polypeptides may be mature polypeptides, as defined herein, and may or may not have an amino-terminal methionine residue, depending on the method by which they are prepared.

The term “HEH4 polypeptide fragment” refers to a polypeptide that comprises a truncation at the amino-terminus (with or without a leader sequence) and/or a truncation at the carboxyl-terminus of the polypeptide as set forth in any of SEQ ID NO: 2, SEQ ID NO: 4, or SEQ ID NO: 5. The term “HEH4 polypeptide fragment” also refers to amino-terminal and/or carboxyl-terminal truncations of HEH4 polypeptide orthologs, HEH4 polypeptide derivatives, or HEH4 polypeptide variants, or to amino-terminal and/or carboxyl-terminal truncations of the polypeptides encoded by HEH4 polypeptide allelic variants or HEH4 polypeptide splice variants. HEH4 polypeptide fragments may result from alternative RNA splicing or from in vivo protease activity. Membrane-bound forms of a HEH4 polypeptide are also contemplated by the present invention. In preferred embodiments, truncations and/or deletions comprise about 10 amino acids, or about 20 amino acids, or about 50 amino acids, or about 75 amino acids, or about 100 amino acids, or more than about 100 amino acids. The polypeptide fragments so produced will comprise about 25 contiguous amino acids, or about 50 amino acids, or about 75 amino acids, or about 100 amino acids, or about 150 amino acids, or about 200 amino acids, or more than about 200 amino acids. Such HEH4 polypeptide fragments may optionally comprise an amino-terminal methionine residue. It will be appreciated that such fragments can be used, for example, to generate antibodies to HEH4 polypeptides.

The term “HEH4 polypeptide ortholog” refers to a polypeptide from another species that corresponds to HEH4 polypeptide amino acid sequence as set forth in any of SEQ ID NO: 2, SEQ ID NO: 4, or SEQ ID NO: 5. For example, mouse and human HEH4 polypeptides are considered orthologs of each other.

The term “HEH4 polypeptide variants” refers to HEH4 polypeptides comprising amino acid sequences having one or more amino acid sequence substitutions, deletions (such as internal deletions and/or HEH4 polypeptide fragments), and/or additions (such as internal additions and/or HEH4 fusion polypeptides) as compared to the HEH4 polypeptide amino acid sequence set forth in any of SEQ ID NO: 2, SEQ ID NO: 4, or SEQ ID NO: 5 (with or without a leader sequence). Variants may be naturally occurring (e.g., HEH4 polypeptide allelic variants, HEH4 polypeptide orthologs, and HEH4 polypeptide splice variants) or artificially constructed. Such HEH4 polypeptide variants may be prepared from the corresponding nucleic acid molecules having a DNA sequence that varies accordingly from the DNA sequence as set forth in either SEQ ID NO: 1 or SEQ ID NO: 3. In preferred embodiments, the variants have from 1 to 3, or from 1 to 5, or from 1 to 10, or from 1 to 15, or from 1 to 20, or from 1 to 25, or from 1 to 50, or from 1 to 75, or from 1 to 100, or more than 100 amino acid substitutions, insertions, additions and/or deletions, wherein the substitutions may be conservative, or non-conservative, or any combination thereof.

The term “HEH4 polypeptide derivatives” refers to the polypeptide as set forth in any of SEQ ID NO: 2, SEQ ID NO: 4, or SEQ ID NO: 5, HEH4 polypeptide fragments, HEH4 polypeptide orthologs, or HEH4 polypeptide variants, as defined herein, that have been chemically modified. The term “HEH4 polypeptide derivatives” also refers to the polypeptides encoded by HEH4 polypeptide allelic variants or HEH4 polypeptide splice variants, as defined herein, which have been chemically modified.

The term “mature HEH4 polypeptide” refers to a HEH4 polypeptide lacking a leader sequence. A mature HEH4 polypeptide may also include other modifications such as proteolytic processing of the amino-terminus (with or without a leader sequence) and/or the carboxyl-terminus, cleavage of a smaller polypeptide from a larger precursor, N-linked and/or O-linked glycosylation, and the like.

The term “HEH4 fusion polypeptide” refers to a fusion of one or more amino acids (such as a heterologous protein or peptide) at the amino- or carboxyl-terminus of the polypeptide as set forth in any of SEQ ID NO: 2, SEQ ID NO: 4, or SEQ ID NO: 5, HEH4 polypeptide fragments, HEH4 polypeptide orthologs, HEH4 polypeptide variants, or HEH4 derivatives, as defined herein. The term “HEH4 fusion polypeptide” also refers to a fusion of one or more amino acids at the amino- or carboxyl-terminus of the polypeptide encoded by HEH4 polypeptide allelic variants or HEH4 polypeptide splice variants, as defined herein.

The term “biologically active HEH4 polypeptides” refers to HEH4 polypeptides having at least one activity characteristic of the polypeptide comprising the amino acid sequence of any of SEQ ID NO: 2, SEQ ID NO: 4, or SEQ ID NO: 5. In addition, a HEH4 polypeptide may be active as an immunogen; that is, the HEH4 polypeptide contains at least one epitope to which antibodies may be raised.

The term “isolated polypeptide” refers to a polypeptide of the present invention that (1) has been separated from at least about 50 percent of polynucleotides, lipids, carbohydrates, or other materials with which it is naturally found when isolated from the source cell, (2) is not linked (by covalent or noncovalent interaction) to all or a portion of a polypeptide to which the “isolated polypeptide” is linked in nature, (3) is operably linked (by covalent or noncovalent interaction) to a polypeptide with which it is not linked in nature, or (4) does not occur in nature. Preferably, the isolated polypeptide is substantially free from any other contaminating polypeptides or other contaminants that are found in its natural environment that would interfere with its therapeutic, diagnostic, prophylactic or research use.

The term “identity,” as known in the art, refers to a relationship between the sequences of two or more polypeptide molecules or two or more nucleic acid molecules, as determined by comparing the sequences. In the art, “identity” also means the degree of sequence relatedness between nucleic acid molecules or polypeptides, as the case may be, as determined by the match between strings of two or more nucleotide or two or more amino acid sequences. “Identity” measures the percent of identical matches between the smaller of two or more sequences with gap alignments (if any) addressed by a particular mathematical model or computer program (i.e., “algorithms”).

The term “similarity” is a related concept, but in contrast to “identity,” “similarity” refers to a measure of relatedness that includes both identical matches and conservative substitution matches. If two polypeptide sequences have, for example, 10/20 identical amino acids, and the remainder are all non-conservative substitutions, then the percent identity and similarity would both be 50%. If in the same example, there are five more positions where there are conservative substitutions, then the percent identity remains 50%, but the percent similarity would be 75% (15/20). Therefore, in cases where there are conservative substitutions, the percent similarity between two polypeptides will be higher than the percent identity between those two polypeptides.

The term “naturally occurring” or “native” when used in connection with biological materials such as nucleic acid molecules, polypeptides, host cells, and the like, refers to materials which are found in nature and are not manipulated by man. Similarly, “non-naturally occurring” or “non-native” as used herein refers to a material that is not found in nature or that has been structurally modified or synthesized by man. When used in connection with nucleotides, the terms “naturally occurring” or “native” refer to the bases adenine (A), cytosine (C), guanine (G), thymine (T), and uracil (U). When used in connection with amino acids, the terms “naturally occurring” and “native” refer to the 20 amino acids alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W), and tyrosine (Y).

The terms “effective amount” and “therapeutically effective amount” each refer to the amount of a HEH4 polypeptide, nucleic acid molecule, or selective binding agent used to support an observable level of one or more biological activities of the HEH4 polypeptides as set forth herein.

The term “pharmaceutically acceptable carrier” or “physiologically acceptable carrier” as used herein refers to one or more formulation materials suitable for accomplishing or enhancing the delivery of the HEH4 polypeptide, HEH4 nucleic acid molecule, or HEH4 selective binding agent as a pharmaceutical composition.

The term “antigen” refers to a molecule or a portion of a molecule capable of being bound by a selective binding agent, such as an antibody, and additionally capable of being used in an animal to produce antibodies capable of binding to an epitope of that antigen. An antigen may have one or more epitopes.

The term “selective binding agent” refers to a molecule or molecules having specificity for a HEH4 polypeptide. As used herein, the terms, “specific” and “specificity” refer to the ability of the selective binding agents to bind to human HEH4 polypeptides and not to bind to human non-HEH4 polypeptides. It will be appreciated, however, that the selective binding agents may also bind orthologs of the polypeptide as set forth in any of SEQ ID NO: 2, SEQ ID NO: 4, or SEQ ID NO: 5, that is, interspecies versions thereof, such as mouse and rat HEH4 polypeptides.

The term “transduction” is used to refer to the transfer of genes from one bacterium to another, usually by a phage. “Transduction” also refers to the acquisition and transfer of eukaryotic cellular sequences by retroviruses.

The term “transfection” is used to refer to the uptake of foreign or exogenous DNA by a cell, and a cell has been “transfected” when the exogenous DNA has been introduced inside the cell membrane. A number of transfection techniques are well known in the art and are disclosed herein. See, e.g., Graham et al., 1973, Virology 52:456; Sambrook et al., Molecular Cloning, A Laboratory Manual (Cold Spring Harbor Laboratories, 1989); Davis et al., Basic Methods in Molecular Biology (Elsevier, 1986); and Chu et al., 1981, Gene 13:197. Such techniques can be used to introduce one or more exogenous DNA moieties into suitable host cells.

The term “transformation” as used herein refers to a change in a cell's genetic characteristics, and a cell has been transformed when it has been modified to contain a new DNA. For example, a cell is transformed where it is genetically modified from its native state. Following transfection or transduction, the transforming DNA may recombine with that of the cell by physically integrating into a chromosome of the cell, may be maintained transiently as an episomal element without being replicated, or may replicate independently as a plasmid. A cell is considered to have been stably transformed when the DNA is replicated with the division of the cell.

Relatedness of Nucleic Acid Molecules and/or Polypeptides

It is understood that related nucleic acid molecules include allelic or splice variants of the nucleic acid molecule of either SEQ ID NO: 1 or SEQ ID NO: 3, and include sequences which are complementary to any of the above nucleotide sequences. Related nucleic acid molecules also include a nucleotide sequence encoding a polypeptide comprising or consisting essentially of a substitution, modification, addition and/or deletion of one or more amino acid residues compared to the polypeptide set forth in any of SEQ ID NO: 2, SEQ ID NO: 4, or SEQ ID NO: 5. Such related HEH4 polypeptides may comprise, for example, an addition and/or a deletion of one or more N-linked or O-linked glycosylation sites or an addition and/or a deletion of one or more cysteine residues.

Related nucleic acid molecules also include fragments of HEH4 nucleic acid molecules which encode a polypeptide of at least about 25 contiguous amino acids, or about 50 amino acids, or about 75 amino acids, or about 100 amino acids, or about 150 amino acids, or about 200 amino acids, or more than about 200 amino acid residues of the HEH4 polypeptide of any of SEQ ID NO: 2, SEQ ID NO: 4, or SEQ ID NO: 5.

In addition, related HEH4 nucleic acid molecules also include those molecules which comprise nucleotide sequences which hybridize under moderately or highly stringent conditions as defined herein with the fully complementary sequence of the HEH4 nucleic acid molecule of either SEQ ID NO: 1 or SEQ ID NO: 3, or of a molecule encoding a polypeptide, which polypeptide comprises the amino acid sequence as shown in any of SEQ ID NO: 2, SEQ ID NO: 4, or SEQ ID NO: 5, or of a nucleic acid fragment as defined herein, or of a nucleic acid fragment encoding a polypeptide as defined herein. Hybridization probes may be prepared using the HEH4 sequences provided herein to screen cDNA, genomic or synthetic DNA libraries for related sequences. Regions of the DNA and/or amino acid sequence of HEH4 polypeptide that exhibit significant identity to known sequences are readily determined using sequence alignment algorithms as described herein and those regions may be used to design probes for screening.

The term “highly stringent conditions” refers to those conditions that are designed to permit hybridization of DNA strands whose sequences are highly complementary, and to exclude hybridization of significantly mismatched DNAs. Hybridization stringency is principally determined by temperature, ionic strength, and the concentration of denaturing agents such as formamide. Examples of “highly stringent conditions” for hybridization and washing are 0.015 M sodium chloride, 0.0015 M sodium citrate at 65-68° C. or 0.015 M sodium chloride, 0.0015 M sodium citrate, and 50% formamide at 42° C. See Sambrook, Fritsch & Maniatis, Molecular Cloning: A Laboratory Manual (2nd ed., Cold Spring Harbor Laboratory, 1989); Anderson et al., Nucleic Acid Hybridisation: A Practical Approach Ch. 4 (IRL Press Limited).

More stringent conditions (such as higher temperature, lower ionic strength, higher formamide, or other denaturing agent) may also be used—however, the rate of hybridization will be affected. Other agents may be included in the hybridization and washing buffers for the purpose of reducing non-specific and/or background hybridization. Examples are 0.1% bovine serum albumin, 0.1% polyvinyl-pyrrolidone, 0.1% sodium pyrophosphate, 0.1% sodium dodecylsulfate, NaDodSO₄, (SDS), ficoll, Denhardt's solution, sonicated salmon sperm DNA (or another non-complementary DNA), and dextran sulfate, although other suitable agents can also be used. The concentration and types of these additives can be changed without substantially affecting the stringency of the hybridization conditions. Hybridization experiments are usually carried out at pH 6.8-7.4; however, at typical ionic strength conditions, the rate of hybridization is nearly independent of pH. See Anderson et al., Nucleic Acid Hybridisation: A Practical Approach Ch. 4 (IRL Press Limited).

Factors affecting the stability of DNA duplex include base composition, length, and degree of base pair mismatch. Hybridization conditions can be adjusted by one skilled in the art in order to accommodate these variables and allow DNAs of different sequence relatedness to form hybrids. The melting temperature of a perfectly matched DNA duplex can be estimated by the following equation:
Tₘ(° C.)=81.5+16.6(log[Na+])+0.41(% G+C)−600/N−0.72(% formamide)
where N is the length of the duplex formed, [Na+] is the molar concentration of the sodium ion in the hybridization or washing solution, % G+C is the percentage of (guanine+cytosine) bases in the hybrid. For imperfectly matched hybrids, the melting temperature is reduced by approximately 1° C. for each 1% mismatch.

The term “moderately stringent conditions” refers to conditions under which a DNA duplex with a greater degree of base pair mismatching than could occur under “highly stringent conditions” is able to form. Examples of typical “moderately stringent conditions” are 0.015 M sodium chloride, 0.0015 M sodium citrate at 50-65° C. or 0.015 M sodium chloride, 0.0015 M sodium citrate, and 20% formamide at 37-50° C. By way of example, “moderately stringent conditions” of 50° C. in 0.015 M sodium ion will allow about a 21% mismatch.

It will be appreciated by those skilled in the art that there is no absolute distinction between “highly stringent conditions” and “moderately stringent conditions.” For example, at 0.015 M sodium ion (no formamide), the melting temperature of perfectly matched long DNA is about 71° C. With a wash at 65° C. (at the same ionic strength), this would allow for approximately a 6% mismatch. To capture more distantly related sequences, one skilled in the art can simply lower the temperature or raise the ionic strength.

A good estimate of the melting temperature in 1M NaCl* for oligonucleotide probes up to about 20 nt is given by:
Tm=2° C. per A-T base pair+4° C. per G-C base pair
*The sodium ion concentration in 6× salt sodium citrate (SSC) is 1M. See Suggs et al., Developmental Biology Using Purified Genes 683 (Brown and Fox, eds., 1981).

High stringency washing conditions for oligonucleotides are usually at a temperature of 0-5° C. below the Tm of the oligonucleotide in 6×SSC, 0.1% SDS.

In another embodiment, related nucleic acid molecules comprise or consist of a nucleotide sequence that is at least about 70 percent identical to the nucleotide sequence as shown in either SEQ ID NO: 1 or SEQ ID NO: 3. In preferred embodiments, the nucleotide sequences are about 75 percent, or about 80 percent, or about 85 percent, or about 90 percent, or about 95, 96, 97, 98, or 99 percent identical to the nucleotide sequence as shown in either SEQ ID NO: 1 or SEQ ID NO: 3. Related nucleic acid molecules encode polypeptides possessing at least one activity of the polypeptide set forth in any of SEQ ID NO: 2, SEQ ID NO: 4, or SEQ ID NO: 5.

Differences in the nucleic acid sequence may result in conservative and/or non-conservative modifications of the amino acid sequence relative to the amino acid sequence of any of SEQ ID NO: 2, SEQ ID NO: 4, or SEQ ID NO: 5.

Conservative modifications to a HEH4 polypeptide will produce a polypeptide having functional and chemical characteristics similar to those of HEH4 polypeptides. In contrast, substantial modifications in the functional and/or chemical characteristics of HEH4 polypeptides may be accomplished by selecting substitutions in the HEH4 polypeptide that differ significantly in their effect on maintaining (a) the structure of the molecular backbone in the area of the substitution, for example, as a sheet or helical conformation, (b) the charge or hydrophobicity of the molecule at the target site, or (c) the bulk of the side chain.

For example, a “conservative amino acid substitution” may involve a substitution of a native amino acid residue with a nonnative residue such that there is little or no effect on the polarity or charge of the amino acid residue at that position. Furthermore, any native residue in the polypeptide may also be substituted with alanine, as has been previously described for “alanine scanning mutagenesis.”

Conservative amino acid substitutions also encompass non-naturally occurring amino acid residues that are typically incorporated by chemical peptide synthesis rather than by synthesis in biological systems. These include peptidomimetics, and other reversed or inverted forms of amino acid moieties.

Naturally occurring residues may be divided into classes based on common side chain properties:
1) hydrophobic: norleucine, Met, Ala, Val, Leu, Ile;
2) neutral hydrophilic: Cys, Ser, Thr;
3) acidic: Asp, Glu;
4) basic: Asn, Gln, His, Lys, Arg;
5) residues that influence chain orientation: Gly, Pro; and
6) aromatic: Trp, Tyr, Phe.

For example, non-conservative substitutions may involve the exchange of a member of one of these classes for a member from another class. Such substituted residues may be introduced into regions of the human HEH4 polypeptide that are homologous with non-human HEH4 polypeptides, or into the non-homologous regions of the molecule.

In making such changes, the hydropathic index of amino acids may be considered. Each amino acid has been assigned a hydropathic index on the basis of its hydrophobicity and charge characteristics. The hydropathic indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte et al., 1982, J. Mol. Biol. 157:105-31). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those that are within ±1 are particularly preferred, and those within ±0.5 are even more particularly preferred.

It is also understood in the art that the substitution of like amino acids can be made effectively on the basis of hydrophilicity, particularly where the biologically functionally equivalent protein or peptide thereby created is intended for use in immunological embodiments, as in the present case. The greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, the substitution of amino acids whose hydrophilicity values are within ±2 is preferred, those that are within ±1 are particularly preferred, and those within ±0.5 are even more particularly preferred. One may also identify epitopes from primary amino acid sequences on the basis of hydrophilicity. These regions are also referred to as “epitopic core regions.”

Desired amino acid substitutions (whether conservative or non-conservative) can be determined by those skilled in the art at the time such substitutions are desired. For example, amino acid substitutions can be used to identify important residues of the HEH4 polypeptide, or to increase or decrease the affinity of the HEH4 polypeptides described herein. Exemplary amino acid substitutions are set forth in Table I.

TABLE I
Amino Acid Substitutions

| Original Residues | Exemplary Substitutions | Preferred Substitutions |
|---|---|---|
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, | Leu |
| | Phe, Norleucine | |
| Leu | Norleucine, Ile, | Ile |
| | Val, Met, Ala, Phe | |
| Lys | Arg, 1,4 Diamino-butyric | Arg |
| | Acid, Gln, Asn | |
| Met | Leu, Phe, Ile | Leu |
| Phe | Leu, Val, Ile, Ala, | Leu |
| | Tyr | |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, | Leu |
| | Ala, Norleucine | |

A skilled artisan will be able to determine suitable HEH4 variants using well-known techniques. For identifying suitable areas of the molecule that may be changed without destroying biological activity, one skilled in the art may target areas not believed to be important for activity. For example, when similar polypeptides with similar activities from the same species or from other species are known, one skilled in the art may compare the amino acid sequence of a HEH4 polypeptide to such similar polypeptides. With such a comparison, one can identify residues and portions of the molecules that are conserved among similar polypeptides. It will be appreciated that changes in areas of the HEH4 molecule that are not conserved relative to such similar polypeptides would be less likely to adversely affect the biological activity and/or structure of a HEH4 polypeptide. One skilled in the art would also know that, even in relatively conserved regions, one may substitute chemically similar amino acids for the naturally occurring residues while retaining activity (conservative amino acid residue substitutions). Therefore, even areas that may be important for biological activity or for structure may be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, one can predict the importance of amino acid residues in a HEH4 polypeptide that correspond to amino acid residues that are important for activity or structure in similar polypeptides. One skilled in the art may opt for chemically similar amino acid substitutions for such predicted important amino acid residues of HEH4 polypeptides.

One skilled in the art can also analyze the three-dimensional structure and amino acid sequence in relation to that structure in similar polypeptides. In view of such information, one skilled in the art may predict the alignment of amino acid residues of HEH4 polypeptide with respect to its three dimensional structure. One skilled in the art may choose not to make radical changes to amino acid residues predicted to be on the surface of the protein, since such residues may be involved in important interactions with other molecules. Moreover, one skilled in the art may generate test variants containing a single amino acid substitution at each amino acid residue. The variants could be screened using activity assays known to those with skill in the art. Such variants could be used to gather information about suitable variants. For example, if one discovered that a change to a particular amino acid residue resulted in destroyed, undesirably reduced, or unsuitable activity, variants with such a change would be avoided. In other words, based on information gathered from such routine experiments, one skilled in the art can readily determine the amino acids where further substitutions should be avoided either alone or in combination with other mutations.

A number of scientific publications have been devoted to the prediction of secondary structure. See Moult, 1996, Curr. Opin. Biotechnol. 7:422-27; Chou et al., 1974, Biochemistry 13:222-45; Chou et al., 1974, Biochemistry 113:211-22; Chou et al., 1978, Adv. Enzymol. Relat. Areas Mol. Biol. 47:45-48; Chou et al., 1978, Ann. Rev. Biochem. 47:251-276; and Chou et al., 1979, Biophys. J. 26:367-84. Moreover, computer programs are currently available to assist with predicting secondary structure. One method of predicting secondary structure is based upon homology modeling. For example, two polypeptides or proteins that have a sequence identity of greater than 30%, or similarity greater than 40%, often have similar structural topologies. The recent growth of the protein structural database (PDB) has provided enhanced predictability of secondary structure, including the potential number of folds within the structure of a polypeptide or protein. See Holm et al., 1999, Nucleic Acids Res. 27:244-47. It has been suggested that there are a limited number of folds in a given polypeptide or protein and that once a critical number of structures have been resolved, structural prediction will become dramatically more accurate (Brenner et al., 1997, Curr. Opin. Struct. Biol. 7:369-76).

Additional methods of predicting secondary structure include “threading” (Jones, 1997, Curr. Opin. Struct. Biol. 7:377-87; Sippl et al., 1996, Structure 4:15-19), “profile analysis” (Bowie et al., 1991, Science, 253:164-70; Gribskov et al., 1990, Methods Enzymol. 183:146-59; Gribskov et al., 1987, Proc. Nat. Acad. Sci. U.S.A. 84:4355-58), and “evolutionary linkage” (See Holm et al., supra, and Brenner et al., supra).

Preferred HEH4 polypeptide variants include glycosylation variants wherein the number and/or type of glycosylation sites have been altered compared to the HEH4 polypeptides of the invention. In one embodiment, HEH4 polypeptide variants comprise a greater or a lesser number of N-linked glycosylation sites than the amino acid sequences of the HEH4 polypeptides of the invention. An N-linked glycosylation site is characterized by the sequence: Asn-X-Ser or Asn-X-Thr, wherein the amino acid residue designated as X may be any amino acid residue except proline. The substitution of amino acid residues to create this sequence provides a potential new site for the addition of an N-linked carbohydrate chain. Alternatively, substitutions that eliminate this sequence will remove an existing N-linked carbohydrate chain. Also provided is a rearrangement of N-linked carbohydrate chains wherein one or more N-linked glycosylation sites (typically those that are naturally occurring) are eliminated and one or more new N-linked sites are created. Additional preferred HEH4 variants include cysteine variants, wherein one or more cysteine residues are deleted or substituted with another amino acid (e.g., serine) as compared to the amino acid sequences of the HEH4 polypeptides of the invention. Cysteine variants are useful when HEH4 polypeptides must be refolded into a biologically active conformation such as after the isolation of insoluble inclusion bodies. Cysteine variants generally have fewer cysteine residues than the native protein, and typically have an even number to minimize interactions resulting from unpaired cysteines.

In other embodiments, HEH4 polypeptide variants comprise an amino acid sequence as set forth in any of SEQ ID NO: 2, SEQ ID NO: 4, or SEQ ID NO: 5 with at least one amino acid insertion and wherein the polypeptide has an activity of the polypeptide set forth in any of SEQ ID NO: 2, SEQ ID NO: 4, or SEQ ID NO: 5, or an amino acid sequence encoding a polypeptide as set forth in any of SEQ ID NO: 2, SEQ ID NO: 4, or SEQ ID NO: 5 with at least one amino acid deletion and wherein the polypeptide has an activity of the polypeptide set forth in any of SEQ ID NO: 2, SEQ ID NO: 4, or SEQ ID NO: 5. HEH4 polypeptide variants also comprise an amino acid sequence as set forth in any of SEQ ID NO: 2, SEQ ID NO: 4, or SEQ ID NO: 5 wherein the polypeptide has a carboxyl- and/or amino-terminal truncation and further wherein the polypeptide has an activity of the polypeptide set forth in any of SEQ ID NO: 2, SEQ ID NO: 4, or SEQ ID NO: 5. HEH4 polypeptide variants further comprise an amino acid sequence as set forth in any of SEQ ID NO: 2, SEQ ID NO: 4, or SEQ ID NO: 5 with at least one modification that is an amino acid substitution, amino acid insertion, amino acid deletion, carboxyl-terminal truncation, or amino-terminal truncation, and wherein the polypeptide has an activity of the polypeptide set forth in any of SEQ ID NO: 2, SEQ ID NO: 4, or SEQ ID NO: 5.

In further embodiments, HEH4 polypeptide variants comprise an amino acid sequence that is at least about 70 percent identical to the amino acid sequence as set forth in any of SEQ ID NO: 2, SEQ ID NO: 4, or SEQ ID NO: 5. In preferred embodiments, HEH4 polypeptide variants comprise an amino acid sequence that is at least about 75 percent, or about 80 percent, or about 85 percent, or about 90 percent, or about 95, 96, 97, 98, or 99 percent identical to the amino acid sequence as set forth in any of SEQ ID NO: 2, SEQ ID NO: 4, or SEQ ID NO: 5. HEH4 polypeptide variants possess at least one activity of the polypeptide set forth in any of SEQ ID NO: 2, SEQ ID NO: 4, or SEQ ID NO: 5.

In addition, HEH4 polypeptides may be fused to a homologous polypeptide to form a homodimer or to a heterologous polypeptide to form a heterodimer. Heterologous peptides and polypeptides include, but are not limited to: an epitope to allow for the detection and/or isolation of a HEH4 fusion polypeptide; a transmembrane receptor protein or a portion thereof, such as an extracellular domain or a transmembrane and intracellular domain; a ligand or a portion thereof which binds to a transmembrane receptor protein; an enzyme or portion thereof which is catalytically active; a polypeptide or peptide which promotes oligomerization, such as a leucine zipper domain; a polypeptide or peptide which increases stability, such as an immunoglobulin constant region; and a polypeptide which has a therapeutic activity different from the HEH4 polypeptides of the present invention.

Fusions can be made either at the amino-terminus or at the carboxyl-terminus of a HEH4 polypeptide. Fusions may be direct with no linker or adapter molecule or may be through a linker or adapter molecule. A linker or adapter molecule may be one or more amino acid residues, typically from about 20 to about 50 amino acid residues. A linker or adapter molecule may also be designed with a cleavage site for a DNA restriction endonuclease or for a protease to allow for the separation of the fused moieties. It will be appreciated that once constructed, the fusion polypeptides can be derivatized according to the methods described herein.

In a further embodiment of the invention, a HEH4 polypeptide is fused to one or more domains of an Fc region of human IgG. Antibodies comprise two functionally independent parts, a variable domain known as “Fab,” that binds an antigen, and a constant domain known as “Fc,” that is involved in effector functions such as complement activation and attack by phagocytic cells. An Fc has a long serum half-life, whereas an Fab is short-lived. Capon et al., 1989, Nature 337:525-31. When constructed together with a therapeutic protein, an Fc domain can provide longer half-life or incorporate such functions as Fc receptor binding, protein A binding, complement fixation, and perhaps even placental transfer. Id. Table II summarizes the use of certain Fc fusions known in the art.

TABLE II
Fc Fusion with Therapeutic Proteins

| Form of Fc | Fusion partner | Therapeutic implications | Reference |
|---|---|---|---|
| IgG1 | N-terminus of | Hodgkin's disease; | U.S. Pat. No. |
| | CD30-L | anaplastic lymphoma; T- | 5,480,981 |
| | | cell leukemia | |
| Murine Fcγ2a | IL-10 | anti-inflammatory; | Zheng et al., 1995, J. |
| | | transplant rejection | Immunol. 154: 5590-600 |
| IgG1 | TNF receptor | septic shock | Fisher et al., 1996, N. |
| | | | Engl. J. Med. 334: 1697-1702; |
| | | | Van Zee et al., |
| | | | 1996, J. Immunol. |
| | | | 156: 2221-30 |
| IgG, IgA, IgM, | TNF receptor | inflammation, | U.S. Pat. No. |
| or IgE | | autoimmune disorders | 5,808,029 |
| (excluding the | | | |
| first domain) | | | |
| IgG1 | CD4 receptor | AIDS | Capon et al., 1989, |
| | | | Nature 337: 525-31 |
| IgG1, | N-terminus | anti-cancer, antiviral | Harvill et al., 1995, |
| IgG3 | of IL-2 | | Immunotech. 1: 95-105 |
| IgG1 | C-terminus of | osteoarthritis; | International Pub. No. |
| | OPG | bone density | WO 97/23614 |
| IgG1 | N-terminus of | anti-obesity | International Pub. No. |
| | leptin | | WO 98/28427 |
| Human Ig Cγ1 | CTLA-4 | autoimmune disorders | Linsley, 1991, J. Exp. |
| | | | Med., 174: 561-69 |

In one example, a human IgG hinge, CH2, and CH3 region may be fused at either the amino-terminus or carboxyl-terminus of the HEH4 polypeptides using methods known to the skilled artisan. In another example, a human IgG hinge, CH2, and CH3 region may be fused at either the amino-terminus or carboxyl-terminus of a HEH4 polypeptide fragment (e.g., the predicted extracellular portion of HEH4 polypeptide).

The resulting HEH4 fusion polypeptide may be purified by use of a Protein A affinity column. Peptides and proteins fused to an Fc region have been found to exhibit a substantially greater half-life in vivo than the unfused counterpart. Also, a fusion to an Fc region allows for dimerization/multimerization of the fusion polypeptide. The Fc region may be a naturally occurring Fc region, or may be altered to improve certain qualities, such as therapeutic qualities, circulation time, or reduced aggregation.

Useful modifications of protein therapeutic agents by fusion with the “Fc” domain of an antibody are discussed in detail in International Pub. No. WO 99/25044, which is hereby incorporated by reference in its entirety. That patent application discusses linkage to a “vehicle” such as polyethylene gycol (PEG), dextran, or an Fc region.

Identity and similarity of related nucleic acid molecules and polypeptides are readily calculated by known methods. Such methods include, but are not limited to those described in Computational Molecular Biology (A. M. Lesk, ed., Oxford University Press 1988); Biocomputing: Informatics and Genome Projects (D. W. Smith, ed., Academic Press 1993); Computer Analysis of Sequence Data (Part 1, A. M. Griffin and H. G. Griffin, eds., Humana Press 1994); G. von Heijne, Sequence Analysis in Molecular Biology (Academic Press 1987); Sequence Analysis Primer (M. Gribskov and J. Devereux, eds., M. Stockton Press 1991); and Carillo et al., 1988, SIAM J. Applied Math., 48:1073.

Preferred methods to determine identity and/or similarity are designed to give the largest match between the sequences tested. Methods to determine identity and similarity are described in publicly available computer programs. Preferred computer program methods to determine identity and similarity between two sequences include, but are not limited to, the GCG program package, including GAP (Devereux et al., 1984, Nucleic Acids Res. 12:387; Genetics Computer Group, University of Wisconsin, Madison, Wis.), BLASTP, BLASTN, and FASTA (Altschul et al., 1990, J. Mol. Biol. 215:403-10). The BLASTX program is publicly available from the National Center for Biotechnology Information (NCBI) and other sources (Altschul et al., BLAST Manual (NCB NLM NIH, Bethesda, Md.); Altschul et al., 1990, supra). The well-known Smith Waterman algorithm may also be used to determine identity.

Certain alignment schemes for aligning two amino acid sequences may result in the matching of only a short region of the two sequences, and this small aligned region may have very high sequence identity even though there is no significant relationship between the two full-length sequences. Accordingly, in a preferred embodiment, the selected alignment method (GAP program) will result in an alignment that spans at least contiguous amino acids of the claimed polypeptide.

For example, using the computer algorithm GAP (Genetics Computer Group, University of Wisconsin, Madison, Wis.), two polypeptides for which the percent sequence identity is to be determined are aligned for optimal matching of their respective amino acids (the “matched span,” as determined by the algorithm). A gap opening penalty (which is calculated as 3× the average diagonal; the “average diagonal” is the average of the diagonal of the comparison matrix being used; the “diagonal” is the score or number assigned to each perfect amino acid match by the particular comparison matrix) and a gap extension penalty (which is usually 0.1× the gap opening penalty), as well as a comparison matrix such as PAM 250 or BLOSUM 62 are used in conjunction with the algorithm. A standard comparison matrix is also used by the algorithm (see Dayhoff et al., 5 Atlas of Protein Sequence and Structure (Supp. 3 1978)(PAM250 comparison matrix); Henikoff et al., 1992, Proc. Natl. Acad. Sci USA 89:10915-19 (BLOSUM 62 comparison matrix)).

Preferred parameters for polypeptide sequence comparison include the following:
Algorithm: Needleman and Wunsch, 1970, J. Mol. Biol. 48:443-53;
Comparison matrix: BLOSUM 62 (Henikoff et al., supra);
Gap Penalty: 12
Gap Length Penalty: 4
Threshold of Similarity: 0
   The GAP program is useful with the above parameters. The aforementioned parameters are the default parameters for polypeptide comparisons (along with no penalty for end gaps) using the GAP algorithm.

Preferred parameters for nucleic acid molecule sequence comparison include the following:
Algorithm: Needleman and Wunsch, supra;
Comparison matrix: matches=+10, mismatch=0
Gap Penalty: 50
Gap Length Penalty: 3
   The GAP program is also useful with the above parameters. The aforementioned parameters are the default parameters for nucleic acid molecule comparisons.

Other exemplary algorithms, gap opening penalties, gap extension penalties, comparison matrices, and thresholds of similarity may be used, including those set forth in the Program Manual, Wisconsin Package, Version 9, September, 1997. The particular choices to be made will be apparent to those of skill in the art and will depend on the specific comparison to be made, such as DNA-to-DNA, protein-to-protein, protein-to-DNA; and additionally, whether the comparison is between given pairs of sequences (in which case GAP or BestFit are generally preferred) or between one sequence and a large database of sequences (in which case FASTA or BLASTA are preferred).

Nucleic Acid Molecules

The nucleic acid molecules encoding a polypeptide comprising the amino acid sequence of a HEH4 polypeptide can readily be obtained in a variety of ways including, without limitation, chemical synthesis, cDNA or genomic library screening, expression library screening, and/or PCR amplification of cDNA.

Recombinant DNA methods used herein are generally those set forth in Sambrook et al., Molecular Cloning: A Laboratory Manual (Cold Spring Harbor Laboratory Press, 1989) and/or Current Protocols in Molecular Biology (Ausubel et al., eds., Green Publishers Inc. and Wiley and Sons 1994). The invention provides for nucleic acid molecules as described herein and methods for obtaining such molecules.

Where a gene encoding the amino acid sequence of a HEH4 polypeptide has been identified from one species, all or a portion of that gene may be used as a probe to identify orthologs or related genes from the same species. The probes or primers may be used to screen cDNA libraries from various tissue sources believed to express the HEH4 polypeptide. In addition, part or all of a nucleic acid molecule having the sequence as set forth in either SEQ ID NO: 1 or SEQ ID NO: 3 may be used to screen a genomic library to identify and isolate a gene encoding the amino acid sequence of a HEH4 polypeptide. Typically, conditions of moderate or high stringency will be employed for screening to minimize the number of false positives obtained from the screening.

Nucleic acid molecules encoding the amino acid sequence of HEH4 polypeptides may also be identified by expression cloning which employs the detection of positive clones based upon a property of the expressed protein. Typically, nucleic acid libraries are screened by the binding of an antibody or other binding partner (e.g., receptor or ligand) to cloned proteins that are expressed and displayed on a host cell surface. The antibody or binding partner is modified with a detectable label to identify those cells expressing the desired clone.

Recombinant expression techniques conducted in accordance with the descriptions set forth below may be followed to produce these polynucleotides and to express the encoded polypeptides. For example, by inserting a nucleic acid sequence that encodes the amino acid sequence of a HEH4 polypeptide into an appropriate vector, one skilled in the art can readily produce large quantities of the desired nucleotide sequence. The sequences can then be used to generate detection probes or amplification primers. Alternatively, a polynucleotide encoding the amino acid sequence of a HEH4 polypeptide can be inserted into an expression vector. By introducing the expression vector into an appropriate host, the encoded HEH4 polypeptide may be produced in large amounts.

Another method for obtaining a suitable nucleic acid sequence is the polymerase chain reaction (PCR). In this method, cDNA is prepared from poly(A)+RNA or total RNA using the enzyme reverse transcriptase. Two primers, typically complementary to two separate regions of cDNA encoding the amino acid sequence of a HEH4 polypeptide, are then added to the cDNA along with a polymerase such as Taq polymerase, and the polymerase amplifies the cDNA region between the two primers.

Another means of preparing a nucleic acid molecule encoding the amino acid sequence of a HEH4 polypeptide is chemical synthesis using methods well known to the skilled artisan such as those described by Engels et al., 1989, Angew. Chem. Intl. Ed. 28:716-34. These methods include, inter alia, the phosphotriester, phosphoramidite, and H-phosphonate methods for nucleic acid synthesis. A preferred method for such chemical synthesis is polymer-supported synthesis using standard phosphoramidite chemistry. Typically, the DNA encoding the amino acid sequence of a HEH4 polypeptide will be several hundred nucleotides in length. Nucleic acids larger than about 100 nucleotides can be synthesized as several fragments using these methods. The fragments can then be ligated together to form the full-length nucleotide sequence of a HEH4 gene. Usually, the DNA fragment encoding the amino-terminus of the polypeptide will have an ATG, which encodes a methionine residue. This methionine may or may not be present on the mature form of the HEH4 polypeptide, depending on whether the polypeptide produced in the host cell is designed to be secreted from that cell. Other methods known to the skilled artisan may be used as well.

In certain embodiments, nucleic acid variants contain codons which have been altered for optimal expression of a HEH4 polypeptide in a given host cell. Particular codon alterations will depend upon the HEH4 polypeptide and host cell selected for expression. Such “codon optimization” can be carried out by a variety of methods, for example, by selecting codons which are preferred for use in highly expressed genes in a given host cell. Computer algorithms that incorporate codon frequency tables such as “Eco_high.Cod” for codon preference of highly expressed bacterial genes may be used and are provided by the University of Wisconsin Package Version 9.0 (Genetics Computer Group, Madison, Wis.). Other useful codon frequency tables include “Celegans_high.cod,” “Celegans_low.cod,” “Drosophila_high.cod,” “Human_high.cod,” “Maize_high.cod,” and “Yeast_high.cod.”

In some cases, it may be desirable to prepare nucleic acid molecules encoding HEH4 polypeptide variants. Nucleic acid molecules encoding variants may be produced using site directed mutagenesis, PCR amplification, or other appropriate methods, where the primer(s) have the desired point mutations (see Sambrook et al., supra, and Ausubel et al., supra, for descriptions of mutagenesis techniques). Chemical synthesis using methods described by Engels et al., supra, may also be used to prepare such variants. Other methods known to the skilled artisan may be used as well.

Vectors and Host Cells

A nucleic acid molecule encoding the amino acid sequence of a HEH4 polypeptide is inserted into an appropriate expression vector using standard ligation techniques. The vector is typically selected to be functional in the particular host cell employed (i.e., the vector is compatible with the host cell machinery such that amplification of the gene and/or expression of the gene can occur). A nucleic acid molecule encoding the amino acid sequence of a HEH4 polypeptide may be amplified/expressed in prokaryotic, yeast, insect (baculovirus systems) and/or eukaryotic host cells. Selection of the host cell will depend in part on whether a HEH4 polypeptide is to be post-translationally modified (e.g., glycosylated and/or phosphorylated). If so, yeast, insect, or mammalian host cells are preferable. For a review of expression vectors, see Meth. Enz., vol. 185 (D. V. Goeddel, ed., Academic Press 1990).

Typically, expression vectors used in any of the host cells will contain sequences for plasmid maintenance and for cloning and expression of exogenous nucleotide sequences. Such sequences, collectively referred to as “flanking sequences” in certain embodiments will typically include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Each of these sequences is discussed below.

Optionally, the vector may contain a “tag”-encoding sequence, i.e., an oligonucleotide molecule located at the 5′ or 3′ end of the HEH4 polypeptide coding sequence; the oligonucleotide sequence encodes polyHis (such as hexaHis), or another “tag” such as FLAG, HA (hemaglutinin influenza virus), or myc for which commercially available antibodies exist. This tag is typically fused to the polypeptide upon expression of the polypeptide, and can serve as a means for affinity purification of the HEH4 polypeptide from the host cell. Affinity purification can be accomplished, for example, by column chromatography using antibodies against the tag as an affinity matrix. Optionally, the tag can subsequently be removed from the purified HEH4 polypeptide by various means such as using certain peptidases for cleavage.

Flanking sequences may be homologous (i.e., from the same species and/or strain as the host cell), heterologous (i.e., from a species other than the host cell species or strain), hybrid (i.e., a combination of flanking sequences from more than one source), or synthetic, or the flanking sequences may be native sequences that normally function to regulate HEH4 polypeptide expression. As such, the source of a flanking sequence may be any prokaryotic or eukaryotic organism, any vertebrate or invertebrate organism, or any plant, provided that the flanking sequence is functional in, and can be activated by, the host cell machinery.

Flanking sequences useful in the vectors of this invention may be obtained by any of several methods well known in the art. Typically, flanking sequences useful herein—other than the HEH4 gene flanking sequences—will have been previously identified by mapping and/or by restriction endonuclease digestion and can thus be isolated from the proper tissue source using the appropriate restriction endonucleases. In some cases, the full nucleotide sequence of a flanking sequence may be known. Here, the flanking sequence may be synthesized using the methods described herein for nucleic acid synthesis or cloning.

Where all or only a portion of the flanking sequence is known, it may be obtained using PCR and/or by screening a genomic library with a suitable oligonucleotide and/or flanking sequence fragment from the same or another species. Where the flanking sequence is not known, a fragment of DNA containing a flanking sequence may be isolated from a larger piece of DNA that may contain, for example, a coding sequence or even another gene or genes. Isolation may be accomplished by restriction endonuclease digestion to produce the proper DNA fragment followed by isolation using agarose gel purification, Qiagen® column chromatography (Chatsworth, Calif.), or other methods known to the skilled artisan. The selection of suitable enzymes to accomplish this purpose will be readily apparent to one of ordinary skill in the art.

An origin of replication is typically a part of those prokaryotic expression vectors purchased commercially, and the origin aids in the amplification of the vector in a host cell. Amplification of the vector to a certain copy number can, in some cases, be important for the optimal expression of a HEH4 polypeptide. If the vector of choice does not contain an origin of replication site, one may be chemically synthesized based on a known sequence, and ligated into the vector. For example, the origin of replication from the plasmid pBR322 (New England Biolabs, Beverly, Mass.) is suitable for most gram-negative bacteria and various origins (e.g., SV40, polyoma, adenovirus, vesicular stomatitus virus (VSV), or papillomaviruses such as HPV or BPV) are useful for cloning vectors in mammalian cells. Generally, the origin of replication component is not needed for mammalian expression vectors (for example, the SV40 origin is often used only because it contains the early promoter).

A transcription termination sequence is typically located 3′ of the end of a polypeptide coding region and serves to terminate transcription. Usually, a transcription termination sequence in prokaryotic cells is a G-C rich fragment followed by a poly-T sequence. While the sequence is easily cloned from a library or even purchased commercially as part of a vector, it can also be readily synthesized using methods for nucleic acid synthesis such as those described herein.

A selectable marker gene element encodes a protein necessary for the survival and growth of a host cell grown in a selective culture medium. Typical selection marker genes encode proteins that (a) confer resistance to antibiotics or other toxins, e.g., ampicillin, tetracycline, or kanamycin for prokaryotic host cells; (b) complement auxotrophic deficiencies of the cell; or (c) supply critical nutrients not available from complex media. Preferred selectable markers are the kanamycin resistance gene, the ampicillin resistance gene, and the tetracycline resistance gene. A neomycin resistance gene may also be used for selection in prokaryotic and eukaryotic host cells.

Other selection genes may be used to amplify the gene that will be expressed. Amplification is the process wherein genes that are in greater demand for the production of a protein critical for growth are reiterated in tandem within the chromosomes of successive generations of recombinant cells. Examples of suitable selectable markers for mammalian cells include dihydrofolate reductase (DHFR) and thymidine kinase. The mammalian cell transformants are placed under selection pressure wherein only the transformants are uniquely adapted to survive by virtue of the selection gene present in the vector. Selection pressure is imposed by culturing the transformed cells under conditions in which the concentration of selection agent in the medium is successively changed, thereby leading to the amplification of both the selection gene and the DNA that encodes a HEH4 polypeptide. As a result, increased quantities of HEH4 polypeptide are synthesized from the amplified DNA.

A ribosome binding site is usually necessary for translation initiation of mRNA and is characterized by a Shine-Dalgarno sequence (prokaryotes) or a Kozak sequence (eukaryotes). The element is typically located 3′ to the promoter and 5′ to the coding sequence of a HEH4 polypeptide to be expressed. The Shine-Dalgarno sequence is varied but is typically a polypurine (i.e., having a high A-G content). Many Shine-Dalgarno sequences have been identified, each of which can be readily synthesized using methods set forth herein and used in a prokaryotic vector.

A leader, or signal, sequence may be used to direct a HEH4 polypeptide out of the host cell. Typically, a nucleotide sequence encoding the signal sequence is positioned in the coding region of a HEH4 nucleic acid molecule, or directly at the 5′ end of a HEH4 polypeptide coding region. Many signal sequences have been identified, and any of those that are functional in the selected host cell may be used in conjunction with a HEH4 nucleic acid molecule. Therefore, a signal sequence may be homologous (naturally occurring) or heterologous to the HEH4 nucleic acid molecule. Additionally, a signal sequence may be chemically synthesized using methods described herein. In most cases, the secretion of a HEH4 polypeptide from the host cell via the presence of a signal peptide will result in the removal of the signal peptide from the secreted HEH4 polypeptide. The signal sequence may be a component of the vector, or it may be a part of a HEH4 nucleic acid molecule that is inserted into the vector.

Included within the scope of this invention is the use of either a nucleotide sequence encoding a native HEH4 polypeptide signal sequence joined to a HEH4 polypeptide coding region or a nucleotide sequence encoding a heterologous signal sequence joined to a HEH4 polypeptide coding region. The heterologous signal sequence selected should be one that is recognized and processed, i.e., cleaved by a signal peptidase, by the host cell. For prokaryotic host cells that do not recognize and process the native HEH4 polypeptide signal sequence, the signal sequence is substituted by a prokaryotic signal sequence selected, for example, from the group of the alkaline phosphatase, penicillinase, or heat-stable enterotoxin II leaders. For yeast secretion, the native HEH4 polypeptide signal sequence may be substituted by the yeast invertase, alpha factor, or acid phosphatase leaders. In mammalian cell expression the native signal sequence is satisfactory, although other mammalian signal sequences may be suitable.

In some cases, such as where glycosylation is desired in a eukaryotic host cell expression system, one may manipulate the various presequences to improve glycosylation or yield. For example, one may alter the peptidase cleavage site of a particular signal peptide, or add pro-sequences, which also may affect glycosylation. The final protein product may have, in the −1 position (relative to the first amino acid of the mature protein) one or more additional amino acids incident to expression, which may not have been totally removed. For example, the final protein product may have one or two amino acid residues found in the peptidase cleavage site, attached to the amino-terminus. Alternatively, use of some enzyme cleavage sites may result in a slightly truncated form of the desired HEH4 polypeptide, if the enzyme cuts at such area within the mature polypeptide.

In many cases, transcription of a nucleic acid molecule is increased by the presence of one or more introns in the vector; this is particularly true where a polypeptide is produced in eukaryotic host cells, especially mammalian host cells. The introns used may be naturally occurring within the HEH4 gene especially where the gene used is a full-length genomic sequence or a fragment thereof. Where the intron is not naturally occurring within the gene (as for most cDNAs), the intron may be obtained from another source. The position of the intron with respect to flanking sequences and the HEH4 gene is generally important, as the intron must be transcribed to be effective. Thus, when a HEH4 cDNA molecule is being transcribed, the preferred position for the intron is 3′ to the transcription start site and 5′ to the poly-A transcription termination sequence. Preferably, the intron or introns will be located on one side or the other (i.e., 5′ or 3′) of the cDNA such that it does not interrupt the coding sequence. Any intron from any source, including viral, prokaryotic and eukaryotic (plant or animal) organisms, may be used to practice this invention, provided that it is compatible with the host cell into which it is inserted. Also included herein are synthetic introns. Optionally, more than one intron may be used in the vector.

The expression and cloning vectors of the present invention will typically contain a promoter that is recognized by the host organism and operably linked to the molecule encoding the HEH4 polypeptide. Promoters are untranscribed sequences located upstream (i.e., 5′) to the start codon of a structural gene (generally within about 100 to 1000 bp) that control the transcription of the structural gene. Promoters are conventionally grouped into one of two classes: inducible promoters and constitutive promoters. Inducible promoters initiate increased levels of transcription from DNA under their control in response to some change in culture conditions, such as the presence or absence of a nutrient or a change in temperature. Constitutive promoters, on the other hand, initiate continual gene product production; that is, there is little or no control over gene expression. A large number of promoters, recognized by a variety of potential host cells, are well known. A suitable promoter is operably linked to the DNA encoding HEH4 polypeptide by removing the promoter from the source DNA by restriction enzyme digestion and inserting the desired promoter sequence into the vector. The native HEH4 promoter sequence may be used to direct amplification and/or expression of a HEH4 nucleic acid molecule. A heterologous promoter is preferred, however, if it permits greater transcription and higher yields of the expressed protein as compared to the native promoter, and if it is compatible with the host cell system that has been selected for use.

Promoters suitable for use with prokaryotic hosts include the beta-lactamase and lactose promoter systems; alkaline phosphatase; a tryptophan (trp) promoter system; and hybrid promoters such as the tac promoter. Other known bacterial promoters are also suitable. Their sequences have been published, thereby enabling one skilled in the art to ligate them to the desired DNA sequence, using linkers or adapters as needed to supply any useful restriction sites.

Suitable promoters for use with yeast hosts are also well known in the art. Yeast enhancers are advantageously used with yeast promoters. Suitable promoters for use with mammalian host cells are well known and include, but are not limited to, those obtained from the genomes of viruses such as polyoma virus, fowlpox virus, adenovirus (such as Adenovirus 2), bovine papilloma virus, avian sarcoma virus, cytomegalovirus, retroviruses, hepatitis-B virus and most preferably Simian Virus 40 (SV40). Other suitable mammalian promoters include heterologous mammalian promoters, for example, heat-shock promoters and the actin promoter.

Additional promoters which may be of interest in controlling HEH4 gene expression include, but are not limited to: the SV40 early promoter region (Bernoist and Chambon, 1981, Nature 290:304-10); the CMV promoter; the promoter contained in the 3′ long terminal repeat of Rous sarcoma virus (Yamamoto, et al., 1980, Cell 22:787-97); the herpes thymidine kinase promoter (Wagner et al., 1981, Proc. Natl. Acad. Sci. U.S.A. 78:1444-45); the regulatory sequences of the metallothionine gene (Brinster et al., 1982, Nature 296:39-42); prokaryotic expression vectors such as the beta-lactamase promoter (Villa-Kamaroff et al., 1978, Proc. Natl. Acad. Sci. U.S.A., 75:3727-31); or the tac promoter (DeBoer et al., 1983, Proc. Natl. Acad. Sci. U.S.A., 80:21-25). Also of interest are the following animal transcriptional control regions, which exhibit tissue specificity and have been utilized in transgenic animals: the elastase I gene control region which is active in pancreatic acinar cells (Swift et al., 1984, Cell 38:639-46; Ornitz et al., 1986, Cold Spring Harbor Symp. Quant. Biol. 50:399-409 (1986); MacDonald, 1987, Hepatology 7:425-515); the insulin gene control region which is active in pancreatic beta cells (Hanahan, 1985, Nature 315:115-22); the immunoglobulin gene control region which is active in lymphoid cells (Grosschedl et al., 1984, Cell 38:647-58; Adames et al., 1985, Nature 318:533-38; Alexander et al., 1987, Mol. Cell. Biol., 7:1436-44); the mouse mammary tumor virus control region which is active in testicular, breast, lymphoid and mast cells (Leder et al., 1986, Cell 45:485-95); the albumin gene control region which is active in liver (Pinkert et al., 1987, Genes and Devel. 1:268-76); the alpha-feto-protein gene control region which is active in liver (Krumlauf et al., 1985, Mol. Cell. Biol., 5:1639-48; Hammer et al., 1987, Science 235:53-58); the alpha 1-antitrypsin gene control region which is active in the liver (Kelsey et al., 1987, Genes and Devel. 1:161-71); the beta-globin gene control region which is active in myeloid cells (Mogram et al., 1985, Nature 315:338-40; Kollias et al., 1986, Cell 46:89-94); the myelin basic protein gene control region which is active in oligodendrocyte cells in the brain (Readhead et al., 1987, Cell 48:703-12); the myosin light chain-2 gene control region which is active in skeletal muscle (Sani, 1985, Nature 314:283-86); and the gonadotropic releasing hormone gene control region which is active in the hypothalamus (Mason et al., 1986, Science 234:1372-78).

An enhancer sequence may be inserted into the vector to increase the transcription of a DNA encoding a HEH4 polypeptide of the present invention by higher eukaryotes. Enhancers are cis-acting elements of DNA, usually about 10-300 bp in length, that act on the promoter to increase transcription. Enhancers are relatively orientation and position independent. They have been found 5′ and 3′ to the transcription unit. Several enhancer sequences available from mammalian genes are known (e.g., globin, elastase, albumin, alpha-feto-protein and insulin). Typically, however, an enhancer from a virus will be used. The SV40 enhancer, the cytomegalovirus early promoter enhancer, the polyoma enhancer, and adenovirus enhancers are exemplary enhancing elements for the activation of eukaryotic promoters. While an enhancer may be spliced into the vector at a position 5′ or 3′ to a HEH4 nucleic acid molecule, it is typically located at a site 5′ from the promoter.

Expression vectors of the invention may be constructed from a starting vector such as a commercially available vector. Such vectors may or may not contain all of the desired flanking sequences. Where one or more of the flanking sequences described herein are not already present in the vector, they may be individually obtained and ligated into the vector. Methods used for obtaining each of the flanking sequences are well known to one skilled in the art.

Preferred vectors for practicing this invention are those that are compatible with bacterial, insect, and mammalian host cells. Such vectors include, inter alia, pCRII, pCR3, and pcDNA3.1 (Invitrogen, Carlsbad, Calif.), pBSH (Stratagene, La Jolla, Calif.), pET15 (Novagen, Madison, Wis.), pGEX (Pharmacia Biotech, Piscataway, N.J.), pEGFP-N2 (Clontech, Palo Alto, Calif.), pETL (BlueBacII, Invitrogen), pDSR-alpha (International Pub. No. WO 90/14363) and pFastBacDual (Gibco-BRL, Grand Island, N.Y.).

Additional suitable vectors include, but are not limited to, cosmids, plasmids, or modified viruses, but it will be appreciated that the vector system must be compatible with the selected host cell. Such vectors include, but are not limited to plasmids such as Bluescript® plasmid derivatives (a high copy number Co1E1-based phagemid; Stratagene Cloning Systems, La Jolla Calif.), PCR cloning plasmids designed for cloning Taq-amplified PCR products (e.g., TOPO™ TA Cloning® Kit, PCR2.1® plasmid derivatives; Invitrogen), and mammalian, yeast or virus vectors such as a baculovirus expression system (pBacPAK plasmid derivatives; Clontech, Palo Alto, Calif.).

After the vector has been constructed and a nucleic acid molecule encoding a HEH4 polypeptide has been inserted into the proper site of the vector, the completed vector may be inserted into a suitable host cell for amplification and/or polypeptide expression. The transformation of an expression vector for a HEH4 polypeptide into a selected host cell may be accomplished by well known methods including methods such as transfection, infection, calcium chloride, electroporation, microinjection, lipofection, DEAE-dextran method, or other known techniques. The method selected will in part be a function of the type of host cell to be used. These methods and other suitable methods are well known to the skilled artisan, and are set forth, for example, in Sambrook et al., supra.

Host cells may be prokaryotic host cells (such as E. coli) or eukaryotic host cells (such as a yeast, insect, or vertebrate cell). The host cell, when cultured under appropriate conditions, synthesizes a HEH4 polypeptide that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule.

A number of suitable host cells are known in the art and many are available from the American Type Culture Collection (ATCC), Manassas, Va. Examples include, but are not limited to, mammalian cells, such as Chinese hamster ovary cells (CHO), CHO DHFR(−) cells (Urlaub et al., 1980, Proc. Natl. Acad. Sci. U.S.A. 97:4216-20), human embryonic kidney (HEK) 293 or 293T cells, or 3T3 cells. The selection of suitable mammalian host cells and methods for transformation, culture, amplification, screening, product production, and purification are known in the art. Other suitable mammalian cell lines, are the monkey COS-1 and COS-7 cell lines, and the CV-1 cell line. Further exemplary mammalian host cells include primate cell lines and rodent cell lines, including transformed cell lines. Normal diploid cells, cell strains derived from in vitro culture of primary tissue, as well as primary explants, are also suitable. Candidate cells may be genotypically deficient in the selection gene, or may contain a dominantly acting selection gene. Other suitable mammalian cell lines include but are not limited to, mouse neuroblastoma N2A cells, HeLa, mouse L-929 cells, 3T3 lines derived from Swiss, Balb-c or NIH mice, BHK or HaK hamster cell lines. Each of these cell lines is known by and available to those skilled in the art of protein expression.

Similarly useful as host cells suitable for the present invention are bacterial cells. For example, the various strains of E. coli (e.g., HB101, DH5α, DH10, and MC1061) are well-known as host cells in the field of biotechnology. Various strains of B. subtilis, Pseudomonas spp., other Bacillus spp., Streptomyces spp., and the like may also be employed in this method.

Many strains of yeast cells known to those skilled in the art are also available as host cells for the expression of the polypeptides of the present invention. Preferred yeast cells include, for example, Saccharomyces cerivisae and Pichia pastoris.

Additionally, where desired, insect cell systems may be utilized in the methods of the present invention. Such systems are described, for example, in Kitts et al., 1993, Biotechniques, 14:810-17; Lucklow, 1993, Curr. Opin. Biotechnol. 4:564-72; and Lucklow et al., 1993, J. Virol., 67:4566-79. Preferred insect cells are Sf-9 and Hi5 (Invitrogen).

One may also use transgenic animals to express glycosylated HEH4 polypeptides. For example, one may use a transgenic milk-producing animal (a cow or goat, for example) and obtain the present glycosylated polypeptide in the animal milk. One may also use plants to produce HEH4 polypeptides, however, in general, the glycosylation occurring in plants is different from that produced in mammalian cells, and may result in a glycosylated product which is not suitable for human therapeutic use.

Polypeptide Production

Host cells comprising a HEH4 polypeptide expression vector may be cultured using standard media well known to the skilled artisan. The media will usually contain all nutrients necessary for the growth and survival of the cells. Suitable media for culturing E. coli cells include, for example, Luria Broth (LB) and/or Terrific Broth (TB). Suitable media for culturing eukaryotic cells include Roswell Park Memorial Institute medium 1640 (RPMI 1640), Minimal Essential Medium (MEM) and/or Dulbecco's Modified Eagle Medium (DMEM), all of which may be supplemented with serum and/or growth factors as necessary for the particular cell line being cultured. A suitable medium for insect cultures is Grace's medium supplemented with yeastolate, lactalbumin hydrolysate, and/or fetal calf serum as necessary.

Typically, an antibiotic or other compound useful for selective growth of transfected or transformed cells is added as a supplement to the media. The compound to be used will be dictated by the selectable marker element present on the plasmid with which the host cell was transformed. For example, where the selectable marker element is kanamycin resistance, the compound added to the culture medium will be kanamycin. Other compounds for selective growth include ampicillin, tetracycline, and neomycin.

The amount of a HEH4 polypeptide produced by a host cell can be evaluated using standard methods known in the art. Such methods include, without limitation, Western blot analysis, SDS-polyacrylamide gel electrophoresis, non-denaturing gel electrophoresis, High Performance Liquid Chromatography (HPLC) separation, immunoprecipitation, and/or activity assays such as DNA binding gel shift assays.

If a HEH4 polypeptide has been designed to be secreted from the host cells, the majority of polypeptide may be found in the cell culture medium. If however, the HEH4 polypeptide is not secreted from the host cells, it will be present in the cytoplasm and/or the nucleus (for eukaryotic host cells) or in the cytosol (for gram-negative bacteria host cells).

For a HEH4 polypeptide situated in the host cell cytoplasm and/or nucleus (for eukaryotic host cells) or in the cytosol (for bacterial host cells), the intracellular material (including inclusion bodies for gram-negative bacteria) can be extracted from the host cell using any standard technique known to the skilled artisan. For example, the host cells can be lysed to release the contents of the periplasm/cytoplasm by French press, homogenization, and/or sonication followed by centrifugation.

If a HEH4 polypeptide has formed inclusion bodies in the cytosol, the inclusion bodies can often bind to the inner and/or outer cellular membranes and thus will be found primarily in the pellet material after centrifugation. The pellet material can then be treated at pH extremes or with a chaotropic agent such as a detergent, guanidine, guanidine derivatives, urea, or urea derivatives in the presence of a reducing agent such as dithiothreitol at alkaline pH or tris carboxyethyl phosphine at acid pH to release, break apart, and solubilize the inclusion bodies. The solubilized HEH4 polypeptide can then be analyzed using gel electrophoresis, immunoprecipitation, or the like. If it is desired to isolate the HEH4 polypeptide, isolation may be accomplished using standard methods such as those described herein and in Marston et al., 1990, Meth. Enz., 182:264-75.

In some cases, a HEH4 polypeptide may not be biologically active upon isolation. Various methods for “refolding” or converting the polypeptide to its tertiary structure and generating disulfide linkages can be used to restore biological activity. Such methods include exposing the solubilized polypeptide to a pH usually above 7 and in the presence of a particular concentration of a chaotrope. The selection of chaotrope is very similar to the choices used for inclusion body solubilization, but usually the chaotrope is used at a lower concentration and is not necessarily the same as chaotropes used for the solubilization. In most cases the refolding/oxidation solution will also contain a reducing agent or the reducing agent plus its oxidized form in a specific ratio to generate a particular redox potential allowing for disulfide shuffling to occur in the formation of the protein's cysteine bridges. Some of the commonly used redox couples include cysteine/cystamine, glutathione (GSH)/dithiobis GSH, cupric chloride, dithiothreitol(DTT)/dithiane DTT, and 2-2-mercaptoethanol(bME)/dithio-b(ME). In many instances, a cosolvent may be used or may be needed to increase the efficiency of the refolding, and the more common reagents used for this purpose include glycerol, polyethylene glycol of various molecular weights, arginine and the like.

If inclusion bodies are not formed to a significant degree upon expression of a HEH4 polypeptide, then the polypeptide will be found primarily in the supernatant after centrifugation of the cell homogenate. The polypeptide may be further isolated from the supernatant using methods such as those described herein.

The purification of a HEH4 polypeptide from solution can be accomplished using a variety of techniques. If the polypeptide has been synthesized such that it contains a tag such as Hexahistidine (HEH4 polypeptide/hexaHis) or other small peptide such as FLAG (Eastman Kodak Co., New Haven, Conn.) or myc (Invitrogen) at either its carboxyl- or amino-terminus, it may be purified in a one-step process by passing the solution through an affinity column where the column matrix has a high affinity for the tag.

For example, polyhistidine binds with great affinity and specificity to nickel. Thus, an affinity column of nickel (such as the Qiagen® nickel columns) can be used for purification of HEH4 polypeptide/polyHis. See, e.g., Current Protocols in Molecular Biology § 10.11.8 (Ausubel et al., eds., Green Publishers Inc. and Wiley and Sons 1993).

Additionally, HEH4 polypeptides may be purified through the use of a monoclonal antibody that is capable of specifically recognizing and binding to a HEH4 polypeptide.

Other suitable procedures for purification include, without limitation, affinity chromatography, immunoaffinity chromatography, ion exchange chromatography, molecular sieve chromatography, HPLC, electrophoresis (including native gel electrophoresis) followed by gel elution, and preparative isoelectric focusing (“Isoprime” machine/technique, Hoefer Scientific, San Francisco, Calif.). In some cases, two or more purification techniques may be combined to achieve increased purity.

HEH4 polypeptides may also be prepared by chemical synthesis methods (such as solid phase peptide synthesis) using techniques known in the art such as those set forth by Merrifield et al., 1963, J. Am. Chem. Soc. 85:2149; Houghten et al., 1985, Proc Natl Acad. Sci. USA 82:5132; and Stewart and Young, Solid Phase Peptide Synthesis (Pierce Chemical Co. 1984). Such polypeptides may be synthesized with or without a methionine on the amino-terminus. Chemically synthesized HEH4 polypeptides may be oxidized using methods set forth in these references to form disulfide bridges. Chemically synthesized HEH4 polypeptides are expected to have comparable biological activity to the corresponding HEH4 polypeptides produced recombinantly or purified from natural sources, and thus may be used interchangeably with a recombinant or natural HEH4 polypeptide.

Another means of obtaining HEH4 polypeptide is via purification from biological samples such as source tissues and/or fluids in which the HEH4 polypeptide is naturally found. Such purification can be conducted using methods for protein purification as described herein. The presence of the HEH4 polypeptide during purification may be monitored, for example, using an antibody prepared against recombinantly produced HEH4 polypeptide or peptide fragments thereof.

A number of additional methods for producing nucleic acids and polypeptides are known in the art, and the methods can be used to produce polypeptides having specificity for HEH4 polypeptide. See, e.g., Roberts et al., 1997, Proc. Natl. Acad. Sci. U.S.A. 94:12297-303, which describes the production of fusion proteins between an mRNA and its encoded peptide. See also, Roberts, 1999, Curr. Opin. Chem. Biol. 3:268-73. Additionally, U.S. Pat. No. 5,824,469 describes methods for obtaining oligonucleotides capable of carrying out a specific biological function. The procedure involves generating a heterogeneous pool of oligonucleotides, each having a 5′ randomized sequence, a central preselected sequence, and a 3′ randomized sequence. The resulting heterogeneous pool is introduced into a population of cells that do not exhibit the desired biological function. Subpopulations of the cells are then screened for those that exhibit a predetermined biological function. From that subpopulation, oligonucleotides capable of carrying out the desired biological function are isolated.

U.S. Pat. Nos. 5,763,192; 5,814,476; 5,723,323; and 5,817,483 describe processes for producing peptides or polypeptides. This is done by producing stochastic genes or fragments thereof, and then introducing these genes into host cells that produce one or more proteins encoded by the stochastic genes. The host cells are then screened to identify those clones producing peptides or polypeptides having the desired activity.

Another method for producing peptides or polypeptides is described in International Pub. No. WO99/15650, filed by Athersys, Inc. Known as “Random Activation of Gene Expression for Gene Discovery” (RAGE-GD), the process involves the activation of endogenous gene expression or over-expression of a gene by in situ recombination methods. For example, expression of an endogenous gene is activated or increased by integrating a regulatory sequence into the target cell that is capable of activating expression of the gene by non-homologous or illegitimate recombination. The target DNA is first subjected to radiation, and a genetic promoter inserted. The promoter eventually locates a break at the front of a gene, initiating transcription of the gene. This results in expression of the desired peptide or polypeptide.

It will be appreciated that these methods can also be used to create comprehensive HEH4 polypeptide expression libraries, which can subsequently be used for high throughput phenotypic screening in a variety of assays, such as biochemical assays, cellular assays, and whole organism assays (e.g., plant, mouse, etc.).

Synthesis

It will be appreciated by those skilled in the art that the nucleic acid and polypeptide molecules described herein may be produced by recombinant and other means.

Selective Binding Agents

The term “selective binding agent” refers to a molecule that has specificity for one or more HEH4 polypeptides. Suitable selective binding agents include, but are not limited to, antibodies and derivatives thereof, polypeptides, and small molecules. Suitable selective binding agents may be prepared using methods known in the art. An exemplary HEH4 polypeptide selective binding agent of the present invention is capable of binding a certain portion of the HEH4 polypeptide thereby inhibiting the binding of the polypeptide to a HEH4 polypeptide binding partner (a ligand or receptor).

Selective binding agents such as antibodies and antibody fragments that bind HEH4 polypeptides are within the scope of the present invention. The antibodies may be polyclonal including monospecific polyclonal; monoclonal (MAbs); recombinant; chimeric; humanized, such as complementarity-determining region (CDR)-grafted; human; single chain; and/or bispecific; as well as fragments; variants; or derivatives thereof. Antibody fragments include those portions of the antibody that bind to an epitope on the HEH4 polypeptide. Examples of such fragments include Fab and F(ab′) fragments generated by enzymatic cleavage of full-length antibodies. Other binding fragments include those generated by recombinant DNA techniques, such as the expression of recombinant plasmids containing nucleic acid sequences encoding antibody variable regions.

Polyclonal antibodies directed toward a HEH4 polypeptide generally are produced in animals (e.g., rabbits or mice) by means of multiple subcutaneous or intraperitoneal injections of HEH4 polypeptide and an adjuvant. It may be useful to conjugate a HEH4 polypeptide to a carrier protein that is immunogenic in the species to be immunized, such as keyhole limpet hemocyanin, serum, albumin, bovine thyroglobulin, or soybean trypsin inhibitor. Also, aggregating agents such as alum are used to enhance the immune response. After immunization, the animals are bled and the serum is assayed for anti-HEH4 antibody titer.

Monoclonal antibodies directed toward HEH4 polypeptides are produced using any method that provides for the production of antibody molecules by continuous cell lines in culture. Examples of suitable methods for preparing monoclonal antibodies include the hybridoma methods of Kohler et al., 1975, Nature 256:495-97 and the human B-cell hybridoma method (Kozbor, 1984, J. Immunol. 133:3001; Brodeur et al., Monoclonal Antibody Production Techniques and Applications 51-63 (Marcel Dekker, Inc., 1987). Also provided by the invention are hybridoma cell lines that produce monoclonal antibodies reactive with HEH4 polypeptides.

Monoclonal antibodies of the invention may be modified for use as therapeutics. One embodiment is a “chimeric” antibody in which a portion of the heavy (H) and/or light (L) chain is identical with or homologous to a corresponding sequence in antibodies derived from a particular species or belonging to a particular antibody class or subclass, while the remainder of the chain(s) is/are identical with or homologous to a corresponding sequence in antibodies derived from another species or belonging to another antibody class or subclass. Also included are fragments of such antibodies, so long as they exhibit the desired biological activity. See U.S. Pat. No. 4,816,567; Morrison et al., 1985, Proc. Natl. Acad. Sci. 81:6851-55.

In another embodiment, a monoclonal antibody of the invention is a “humanized” antibody. Methods for humanizing non-human antibodies are well known in the art. See U.S. Pat. Nos. 5,585,089 and 5,693,762. Generally, a humanized antibody has one or more amino acid residues introduced into it from a source that is non-human. Humanization can be performed, for example, using methods described in the art (Jones et al., 1986, Nature 321:522-25; Riechmann et al., 1998, Nature 332:323-27; Verhoeyen et al., 1988, Science 239:1534-36), by substituting at least a portion of a rodent complementarity-determining region for the corresponding regions of a human antibody.

Also encompassed by the invention are human antibodies that bind HEH4 polypeptides. Using transgenic animals (e.g., mice) that are capable of producing a repertoire of human antibodies in the absence of endogenous immunoglobulin production such antibodies are produced by immunization with a HEH4 polypeptide antigen (i.e., having at least 6 contiguous amino acids), optionally conjugated to a carrier. See, e.g., Jakobovits et al., 1993, Proc. Natl. Acad. Sci. 90:2551-55; Jakobovits et al., 1993, Nature 362:255-58; Bruggermann et al., 1993, Year in Immuno. 7:33. In one method, such transgenic animals are produced by incapacitating the endogenous loci encoding the heavy and light immunoglobulin chains therein, and inserting loci encoding human heavy and light chain proteins into the genome thereof. Partially modified animals, i.e., animals having less than the full complement of modifications, are then cross-bred to obtain an animal having all of the desired immune system modifications. When administered an immunogen, these transgenic animals produce antibodies with human (rather than, e.g., murine) amino acid sequences, including variable regions that are immunospecific for these antigens. See International Pub. Nos. WO 96/33735 and WO 94/02602. Additional methods are described in U.S. Pat. No. 5,545,807, International Pub. Nos. WO 91/10741 and WO 90/04036, and in European Patent Nos. 546073B1 and 546073A1. Human antibodies can also be produced by the expression of recombinant DNA in host cells or by expression in hybridoma cells as described herein.

In an alternative embodiment, human antibodies can also be produced from phage-display libraries (Hoogenboom et al., 1991, J. Mol. Biol. 227:381; Marks et al., 1991, J. Mol. Biol. 222:581). These processes mimic immune selection through the display of antibody repertoires on the surface of filamentous bacteriophage, and subsequent selection of phage by their binding to an antigen of choice. One such technique is described in International Pub. No. WO 99/10494, which describes the isolation of high affinity and functional agonistic antibodies for MPL- and msk- receptors using such an approach.

Chimeric, CDR grafted, and humanized antibodies are typically produced by recombinant methods. Nucleic acids encoding the antibodies are introduced into host cells and expressed using materials and procedures described herein. In a preferred embodiment, the antibodies are produced in mammalian host cells, such as CHO cells. Monoclonal (e.g., human) antibodies may be produced by the expression of recombinant DNA in host cells or by expression in hybridoma cells as described herein.

The anti-HEH4 antibodies of the invention may be employed in any known assay method, such as competitive binding assays, direct and indirect sandwich assays, and immunoprecipitation assays (Sola, Monoclonal Antibodies: A Manual of Techniques 147-158 (CRC Press, Inc., 1987)) for the detection and quantitation of HEH4 polypeptides. The antibodies will bind HEH4 polypeptides with an affinity that is appropriate for the assay method being employed.

For diagnostic applications, in certain embodiments, anti-HEH4 antibodies may be labeled with a detectable moiety. The detectable moiety can be any one that is capable of producing, either directly or indirectly, a detectable signal. For example, the detectable moiety may be a radioisotope, such as ³H, ¹⁴C, ³²P, ³⁵S, ¹²⁵I, ⁹⁹Tc, ¹¹¹In, or ⁶⁷Ga; a fluorescent or chemiluminescent compound, such as fluorescein isothiocyanate, rhodamine, or luciferin; or an enzyme, such as alkaline phosphatase, β-galactosidase, or horseradish peroxidase (Bayer, et al., 1990, Meth. Enz. 184:138-63).

Competitive binding assays rely on the ability of a labeled standard (e.g., a HEH4 polypeptide, or an immunologically reactive portion thereof) to compete with the test sample analyte (an HEH4 polypeptide) for binding with a limited amount of anti-HEH4 antibody. The amount of a HEH4 polypeptide in the test sample is inversely proportional to the amount of standard that becomes bound to the antibodies. To facilitate determining the amount of standard that becomes bound, the antibodies typically are insolubilized before or after the competition, so that the standard and analyte that are bound to the antibodies may conveniently be separated from the standard and analyte that remain unbound.

Sandwich assays typically involve the use of two antibodies, each capable of binding to a different immunogenic portion, or epitope, of the protein to be detected and/or quantitated. In a sandwich assay, the test sample analyte is typically bound by a first antibody that is immobilized on a solid support, and thereafter a second antibody binds to the analyte, thus forming an insoluble three-part complex. See, e.g., U.S. Pat. No. 4,376,110. The second antibody may itself be labeled with a detectable moiety (direct sandwich assays) or may be measured using an anti-immunoglobulin antibody that is labeled with a detectable moiety (indirect sandwich assays). For example, one type of sandwich assay is an enzyme-linked immunosorbent assay (ELISA), in which case the detectable moiety is an enzyme.

The selective binding agents, including anti-HEH4 antibodies, are also useful for in vivo imaging. An antibody labeled with a detectable moiety may be administered to an animal, preferably into the bloodstream, and the presence and location of the labeled antibody in the host assayed. The antibody may be labeled with any moiety that is detectable in an animal, whether by nuclear magnetic resonance, radiology, or other detection means known in the art.

Selective binding agents of the invention, including antibodies, may be used as therapeutics. These therapeutic agents are generally agonists or antagonists, in that they either enhance or reduce, respectively, at least one of the biological activities of a HEH4 polypeptide. In one embodiment, antagonist antibodies of the invention are antibodies or binding fragments thereof which are capable of specifically binding to a HEH4 polypeptide and which are capable of inhibiting or eliminating the functional activity of a HEH4 polypeptide in vivo or in vitro. In preferred embodiments, the selective binding agent, e.g., an antagonist antibody, will inhibit the functional activity of a HEH4 polypeptide by at least about 50%, and preferably by at least about 80%. In another embodiment, the selective binding agent may be an anti-HEH4 polypeptide antibody that is capable of interfering with the interaction between HEH4 and a HEH4 polypeptide binding partner (a ligand or receptor) thereby inhibiting or eliminating HEH4 polypeptide activity in vitro or in vivo. Selective binding agents, including agonist and antagonist anti-HEH4 polypeptide antibodies, are identified by screening assays that are well known in the art.

The invention also relates to a kit comprising HEH4 selective binding agents (such as antibodies) and other reagents useful for detecting HEH4 polypeptide levels in biological samples. Such reagents may include a detectable label, blocking serum, positive and negative control samples, and detection reagents.

Microarrays

It will be appreciated that DNA microarray technology can be utilized in accordance with the present invention. DNA microarrays are miniature, high-density arrays of nucleic acids positioned on a solid support, such as glass. Each cell or element within the array contains numerous copies of a single nucleic acid species that acts as a target for hybridization with a complementary nucleic acid sequence (e.g., mRNA). In expression profiling using DNA microarray technology, mRNA is first extracted from a cell or tissue sample and then converted enzymatically to fluorescently labeled cDNA. This material is hybridized to the microarray and unbound cDNA is removed by washing. The expression of discrete genes represented on the array is then visualized by quantitating the amount of labeled cDNA that is specifically bound to each target nucleic acid molecule. In this way, the expression of thousands of genes can be quantitated in a high throughput, parallel manner from a single sample of biological material.

This high throughput expression profiling has a broad range of applications with respect to the HEH4 molecules of the invention, including, but not limited to: the identification and validation of HEH4 disease-related genes as targets for therapeutics; molecular toxicology of related HEH4 molecules and inhibitors thereof; stratification of populations and generation of surrogate markers for clinical trials; and enhancing related HEH4 polypeptide small molecule drug discovery by aiding in the identification of selective compounds in high throughput screens.

Chemical Derivatives

Chemically modified derivatives of HEH4 polypeptides may be prepared by one skilled in the art, given the disclosures described herein. HEH4 polypeptide derivatives are modified in a manner that is different—either in the type or location of the molecules naturally attached to the polypeptide. Derivatives may include molecules formed by the deletion of one or more naturally-attached chemical groups. HEH4 polypeptides may be modified by the covalent attachment of one or more polymers. For example, the polymer selected is typically water-soluble so that the protein to which it is attached does not precipitate in an aqueous environment, such as a physiological environment. Included within the scope of suitable polymers is a mixture of polymers. Preferably, for therapeutic use of the end-product preparation, the polymer will be pharmaceutically acceptable.

The polymers each may be of any molecular weight and may be branched or unbranched. The polymers each typically have an average molecular weight of between about 2 kDa to about 100 kDa (the term “about” indicating that in preparations of a water-soluble polymer, some molecules will weigh more, some less, than the stated molecular weight). The average molecular weight of each polymer is preferably between about 5 kDa and about 50 kDa, more preferably between about 12 kDa and about 40 kDa and most preferably between about 20 kDa and about 35 kDa.

Suitable water-soluble polymers or mixtures thereof include, but are not limited to, N-linked or O-linked carbohydrates, sugars, phosphates, polyethylene glycol (PEG) (including the forms of PEG that have been used to derivatize proteins, including mono-(C₁-C₁₀), alkoxy-, or aryloxy-polyethylene glycol), monomethoxy-polyethylene glycol, dextran (such as low molecular weight dextran of, for example, about 6 kD), cellulose, or other carbohydrate based polymers, poly-(N-vinyl pyrrolidone) polyethylene glycol, propylene glycol homopolymers, polypropylene oxide/ethylene oxide co-polymers, polyoxyethylated polyols (e.g., glycerol), and polyvinyl alcohol. Also encompassed by the present invention are bifunctional crosslinking molecules that may be used to prepare covalently attached HEH4 polypeptide multimers.

In general, chemical derivatization may be performed under any suitable condition used to react a protein with an activated polymer molecule. Methods for preparing chemical derivatives of polypeptides will generally comprise the steps of: (a) reacting the polypeptide with the activated polymer molecule (such as a reactive ester or aldehyde derivative of the polymer molecule) under conditions whereby a HEH4 polypeptide becomes attached to one or more polymer molecules, and (b) obtaining the reaction products. The optimal reaction conditions will be determined based on known parameters and the desired result. For example, the larger the ratio of polymer molecules to protein, the greater the percentage of attached polymer molecule. In one embodiment, the HEH4 polypeptide derivative may have a single polymer molecule moiety at the amino-terminus. See, e.g., U.S. Pat. No. 5,234,784.

The pegylation of a polypeptide may be specifically carried out using any of the pegylation reactions known in the art. Such reactions are described, for example, in the following references: Francis et al., 1992, Focus on Growth Factors 3:4-10; European Patent Nos. 0154316 and 0401384; and U.S. Pat. No. 4,179,337. For example, pegylation may be carried out via an acylation reaction or an alkylation reaction with a reactive polyethylene glycol molecule (or an analogous reactive water-soluble polymer) as described herein. For the acylation reactions, a selected polymer should have a single reactive ester group. For reductive alkylation, a selected polymer should have a single reactive aldehyde group. A reactive aldehyde is, for example, polyethylene glycol propionaldehyde, which is water stable, or mono C₁-C₁₀ alkoxy or aryloxy derivatives thereof (see U.S. Pat. No. 5,252,714).

In another embodiment, HEH4 polypeptides may be chemically coupled to biotin. The biotin/HEH4 polypeptide molecules are then allowed to bind to avidin, resulting in tetravalent avidin/biotin/HEH4 polypeptide molecules. HEH4 polypeptides may also be covalently coupled to dinitrophenol (DNP) or trinitrophenol (TNP) and the resulting conjugates precipitated with anti-DNP or anti-TNP-IgM to form decameric conjugates with a valency of 10.

Generally, conditions that may be alleviated or modulated by the administration of the present HEH4 polypeptide derivatives include those described herein for HEH4 polypeptides. However, the HEH4 polypeptide derivatives disclosed herein may have additional activities, enhanced or reduced biological activity, or other characteristics, such as increased or decreased half-life, as compared to the non-derivatized molecules.

Genetically Engineered Non-Human Animals

Additionally included within the scope of the present invention are non-human animals such as mice, rats, or other rodents; rabbits, goats, sheep, or other farm animals, in which the genes encoding native HEH4 polypeptide have been disrupted (i.e., “knocked out”) such that the level of expression of HEH4 polypeptide is significantly decreased or completely abolished. Such animals may be prepared using techniques and methods such as those described in U.S. Pat. No. 5,557,032.

The present invention further includes non-human animals such as mice, rats, or other rodents; rabbits, goats, sheep, or other farm animals, in which either the native form of a HEH4 gene for that animal or a heterologous HEH4 gene is over-expressed by the animal, thereby creating a “transgenic” animal. Such transgenic animals may be prepared using well known methods such as those described in U.S. Pat. No 5,489,743 and International Pub. No. WO 94/28122.

The present invention further includes non-human animals in which the promoter for one or more of the HEH4 polypeptides of the present invention is either activated or inactivated (e.g., by using homologous recombination methods) to alter the level of expression of one or more of the native HEH4 polypeptides.

These non-human animals may be used for drug candidate screening. In such screening, the impact of a drug candidate on the animal may be measured. For example, drug candidates may decrease or increase the expression of the HEH4 gene. In certain embodiments, the amount of HEH4 polypeptide that is produced may be measured after the exposure of the animal to the drug candidate. Additionally, in certain embodiments, one may detect the actual impact of the drug candidate on the animal. For example, over-expression of a particular gene may result in, or be associated with, a disease or pathological condition. In such cases, one may test a drug candidate's ability to decrease expression of the gene or its ability to prevent or inhibit a pathological condition. In other examples, the production of a particular metabolic product such as a fragment of a polypeptide, may result in, or be associated with, a disease or pathological condition. In such cases, one may test a drug candidate's ability to decrease the production of such a metabolic product or its ability to prevent or inhibit a pathological condition.

Assaying for Other Modulators of HEH4 Polypeptide Activity

In some situations, it may be desirable to identify molecules that are modulators, i.e., agonists or antagonists, of the activity of HEH4 polypeptide. Natural or synthetic molecules that modulate HEH4 polypeptide may be identified using one or more screening assays, such as those described herein. Such molecules may be administered either in an ex vivo manner or in an in vivo manner by injection, or by oral delivery, implantation device, or the like.

“Test molecule” refers to a molecule that is under evaluation for the ability to modulate (i.e., increase or decrease) the activity of a HEH4 polypeptide. Most commonly, a test molecule will interact directly with a HEH4 polypeptide. However, it is also contemplated that a test molecule may also modulate HEH4 polypeptide activity indirectly, such as by affecting HEH4 gene expression, or by binding to a HEH4 polypeptide binding partner (e.g., receptor or ligand). In one embodiment, a test molecule will bind to a HEH4 polypeptide with an affinity constant of at least about 10⁻⁶ M, preferably about 10⁻⁸ M, more preferably about 10⁻⁹ M, and even more preferably about 10⁻¹⁰ M.

Methods for identifying compounds that interact with HEH4 polypeptides are encompassed by the present invention. In certain embodiments, a HEH4 polypeptide is incubated with a test molecule under conditions that permit the interaction of the test molecule with a HEH4 polypeptide, and the extent of the interaction is measured. The test molecule can be screened in a substantially purified form or in a crude mixture.

In certain embodiments, a HEH4 polypeptide agonist or antagonist may be a protein, peptide, carbohydrate, lipid, or small molecular weight molecule that interacts with a HEH4 polypeptide to regulate its activity. Molecules which regulate HEH4 polypeptide expression include nucleic acids which are complementary to nucleic acids encoding a HEH4 polypeptide, or are complementary to nucleic acids sequences which direct or control the expression of HEH4 polypeptide, and which act as anti-sense regulators of expression.

Once a test molecule has been identified as interacting with a HEH4 polypeptide, the molecule may be further evaluated for its ability to increase or decrease HEH4 polypeptide activity. The measurement of the interaction of a test molecule with a HEH4 polypeptide may be carried out in several formats, including cell-based binding assays, membrane binding assays, solution-phase assays, and immunoassays. In general, a test molecule is incubated with a HEH4 polypeptide for a specified period of time, and HEH4 polypeptide activity is determined by one or more assays for measuring biological activity.

The interaction of test molecules with HEH4 polypeptides may also be assayed directly using polyclonal or monoclonal antibodies in an immunoassay. Alternatively, modified forms of HEH4 polypeptides containing epitope tags as described herein may be used in solution and immunoassays.

In the event that HEH4 polypeptides display biological activity through an interaction with a binding partner (e.g., a receptor or a ligand), a variety of in vitro assays may be used to measure the binding of a HEH4 polypeptide to the corresponding binding partner (such as a selective binding agent, receptor, or ligand). These assays may be used to screen test molecules for their ability to increase or decrease the rate and/or the extent of binding of a HEH4 polypeptide to its binding partner. In one assay, a HEH4 polypeptide is immobilized in the wells of a microtiter plate. Radiolabeled HEH4 polypeptide binding partner (for example, iodinated HEH4 polypeptide binding partner) and a test molecule can then be added either one at a time (in either order) or simultaneously to the wells. After incubation, the wells can be washed and counted for radioactivity, using a scintillation counter, to determine the extent to which the binding partner bound to the HEH4 polypeptide. Typically, a molecule will be tested over a range of concentrations, and a series of control wells lacking one or more elements of the test assays can be used for accuracy in the evaluation of the results. An alternative to this method involves reversing the “positions” of the proteins, i.e., immobilizing HEH4 polypeptide binding partner to the microtiter plate wells, incubating with the test molecule and radiolabeled HEH4 polypeptide, and determining the extent of HEH4 polypeptide binding. See, e.g., Current Protocols in Molecular Biology, chap. 18 (Ausubel et al., eds., Green Publishers Inc. and Wiley and Sons 1995).

As an alternative to radiolabeling, a HEH4 polypeptide or its binding partner may be conjugated to biotin, and the presence of biotinylated protein can then be detected using streptavidin linked to an enzyme, such as horse radish peroxidase (HRP) or alkaline phosphatase (AP), which can be detected colorometrically, or by fluorescent tagging of streptavidin. An antibody directed to a HEH4 polypeptide or to a HEH4 polypeptide binding partner, and which is conjugated to biotin, may also be used for purposes of detection following incubation of the complex with enzyme-linked streptavidin linked to AP or HRP.

A HEH4 polypeptide or a HEH4 polypeptide binding partner can also be immobilized by attachment to agarose beads, acrylic beads, or other types of such inert solid phase substrates. The substrate-protein complex can be placed in a solution containing the complementary protein and the test compound. After incubation, the beads can be precipitated by centrifugation, and the amount of binding between a HEH4 polypeptide and its binding partner can be assessed using the methods described herein. Alternatively, the substrate-protein complex can be immobilized in a column with the test molecule and complementary protein passing through the column. The formation of a complex between a HEH4 polypeptide and its binding partner can then be assessed using any of the techniques described herein (e.g., radiolabelling or antibody binding).

Another in vitro assay that is useful for identifying a test molecule which increases or decreases the formation of a complex between a HEH4 polypeptide binding protein and a HEH4 polypeptide binding partner is a surface plasmon resonance detector system such as the BIAcore assay system (Pharmacia, Piscataway, N.J.). The BIAcore system is utilized as specified by the manufacturer. This assay essentially involves the covalent binding of either HEH4 polypeptide or a HEH4 polypeptide binding partner to a dextran-coated sensor chip that is located in a detector. The test compound and the other complementary protein can then be injected, either simultaneously or sequentially, into the chamber containing the sensor chip. The amount of complementary protein that binds can be assessed based on the change in molecular mass that is physically associated with the dextran-coated side of the sensor chip, with the change in molecular mass being measured by the detector system.

In some cases, it may be desirable to evaluate two or more test compounds together for their ability to increase or decrease the formation of a complex between a HEH4 polypeptide and a HEH4 polypeptide binding partner. In these cases, the assays set forth herein can be readily modified by adding such additional test compound(s) either simultaneously with, or subsequent to, the first test compound. The remainder of the steps in the assay are as set forth herein.

In vitro assays such as those described herein may be used advantageously to screen large numbers of compounds for an effect on the formation of a complex between a HEH4 polypeptide and HEH4 polypeptide binding partner. The assays may be automated to screen compounds generated in phage display, synthetic peptide, and chemical synthesis libraries.

Compounds which increase or decrease the formation of a complex between a HEH4 polypeptide and a HEH4 polypeptide binding partner may also be screened in cell culture using cells and cell lines expressing either HEH4 polypeptide or HEH4 polypeptide binding partner. Cells and cell lines may be obtained from any mammal, but preferably will be from human or other primate, canine, or rodent sources. The binding of a HEH4 polypeptide to cells expressing HEH4 polypeptide binding partner at the surface is evaluated in the presence or absence of test molecules, and the extent of binding may be determined by, for example, flow cytometry using a biotinylated antibody to a HEH4 polypeptide binding partner. Cell culture assays can be used advantageously to further evaluate compounds that score positive in protein binding assays described herein.

Cell cultures can also be used to screen the impact of a drug candidate. For example, drug candidates may decrease or increase the expression of the HEH4 gene. In certain embodiments, the amount of HEH4 polypeptide or a HEH4 polypeptide fragment that is produced may be measured after exposure of the cell culture to the drug candidate. In certain embodiments, one may detect the actual impact of the drug candidate on the cell culture. For example, the over-expression of a particular gene may have a particular impact on the cell culture. In such cases, one may test a drug candidate's ability to increase or decrease the expression of the gene or its ability to prevent or inhibit a particular impact on the cell culture. In other examples, the production of a particular metabolic product such as a fragment of a polypeptide, may result in, or be associated with, a disease or pathological condition. In such cases, one may test a drug candidate's ability to decrease the production of such a metabolic product in a cell culture.

Internalizing Proteins

The tat protein sequence (from HIV) can be used to internalize proteins into a cell. See, e.g., Falwell et al., 1994, Proc. Natl. Acad. Sci. U.S.A. 91:664-68. For example, an 11 amino acid sequence (Y-G-R-K-K-R-R-Q-R-R-R; SEQ ID NO: 6) of the HIV tat protein (termed the “protein transduction domain,” or TAT PDT) has been described as mediating delivery across the cytoplasmic membrane and the nuclear membrane of a cell. See Schwarze et al., 1999, Science 285:1569-72; and Nagahara et al., 1998, Nat. Med. 4:1449-52. In these procedures, FITC-constructs (FITC-labeled G-G-G-G-Y-G-R-K-K-R-R-Q-R-R-R; SEQ ID NO: 7), which penetrate tissues following intraperitoneal administration, are prepared, and the binding of such constructs to cells is detected by fluorescence-activated cell sorting (FACS) analysis. Cells treated with a tat-β-gal fusion protein will demonstrate β-gal activity. Following injection, expression of such a construct can be detected in a number of tissues, including liver, kidney, lung, heart, and brain tissue. It is believed that such constructs undergo some degree of unfolding in order to enter the cell, and as such, may require a refolding following entry into the cell.

It will thus be appreciated that the tat protein sequence may be used to internalize a desired polypeptide into a cell. For example, using the tat protein sequence, a HEH4 antagonist (such as an anti-HEH4 selective binding agent, small molecule, soluble receptor, or antisense oligonucleotide) can be administered intracellularly to inhibit the activity of a HEH4 molecule. As used herein, the term “HEH4 molecule” refers to both HEH4 nucleic acid molecules and HEH4 polypeptides as defined herein. Where desired, the HEH4 protein itself may also be internally administered to a cell using these procedures. See also, Straus, 1999, Science 285:1466-67.

Compositions of HEH4 Molecules or Selective Binding Agents and Administration

Therapeutic compositions are within the scope of the present invention. Such HEH4 polypeptide pharmaceutical compositions may comprise a therapeutically effective amount of a HEH4 polypeptide or a HEH4 nucleic acid molecule in admixture with a pharmaceutically or physiologically acceptable formulation agent selected for suitability with the mode of administration. Pharmaceutical compositions may comprise a therapeutically effective amount of one or more HEH4 polypeptide selective binding agents in admixture with a pharmaceutically or physiologically acceptable formulation agent selected for suitability with the mode of administration.

Acceptable formulation materials preferably are nontoxic to recipients at the dosages and concentrations employed.

The pharmaceutical composition may contain formulation materials for modifying, maintaining, or preserving, for example, the pH, osmolarity, viscosity, clarity, color, isotonicity, odor, sterility, stability, rate of dissolution or release, adsorption, or penetration of the composition. Suitable formulation materials include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as polysorbate 20 or polysorbate 80; triton; tromethamine; lecithin; cholesterol or tyloxapal), stability enhancing agents (such as sucrose or sorbitol), tonicity enhancing agents (such as alkali metal halides—preferably sodium or potassium chloride—or mannitol sorbitol), delivery vehicles, diluents, excipients and/or pharmaceutical adjuvants. See Remington's Pharmaceutical Sciences (18th Ed., A. R. Gennaro, ed., Mack Publishing Company 1990.

The optimal pharmaceutical composition will be determined by a skilled artisan depending upon, for example, the intended route of administration, delivery format, and desired dosage. See, e.g., Remington's Pharmaceutical Sciences, supra. Such compositions may influence the physical state, stability, rate of in vivo release, and rate of in vivo clearance of the HEH4 molecule or HEH4 selective bind agent.

The primary vehicle or carrier in a pharmaceutical composition may be either aqueous or non-aqueous in nature. For example, a suitable vehicle or carrier for injection may be water, physiological saline solution, or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which may further include sorbitol or a suitable substitute. In one embodiment of the present invention, HEH4 molecule or HEH4 selective bind agent compositions may be prepared for storage by mixing the selected composition having the desired degree of purity with optional formulation agents (Remington's Pharmaceutical Sciences, supra) in the form of a lyophilized cake or an aqueous solution. Further, the HEH4 molecule or HEH4 selective bind agent product may be formulated as a lyophilizate using appropriate excipients such as sucrose.

The HEH4 molecule or HEH4 selective bind agent pharmaceutical compositions can be selected for parenteral delivery. Alternatively, the compositions may be selected for inhalation or for delivery through the digestive tract, such as orally. The preparation of such pharmaceutically acceptable compositions is within the skill of the art.

The formulation components are present in concentrations that are acceptable to the site of administration. For example, buffers are used to maintain the composition at physiological pH or at a slightly lower pH, typically within a pH range of from about 5 to about 8.

When parenteral administration is contemplated, the therapeutic compositions for use in this invention may be in the form of a pyrogen-free, parenterally acceptable, aqueous solution comprising the desired HEH4 molecule or HEH4 selective bind agent in a pharmaceutically acceptable vehicle. A particularly suitable vehicle for parenteral injection is sterile distilled water in which a HEH4 molecule or HEH4 selective bind agent is formulated as a sterile, isotonic solution, properly preserved. Yet another preparation can involve the formulation of the desired molecule with an agent, such as injectable microspheres, bio-erodible particles, polymeric compounds (such as polylactic acid or polyglycolic acid), beads, or liposomes, that provides for the controlled or sustained release of the product which may then be delivered via a depot injection. Hyaluronic acid may also be used, and this may have the effect of promoting sustained duration in the circulation. Other suitable means for the introduction of the desired molecule include implantable drug delivery devices.

In one embodiment, a pharmaceutical composition may be formulated for inhalation. For example, HEH4 molecule or HEH4 selective bind agent may be formulated as a dry powder for inhalation. HEH4 molecule or HEH4 selective bind agent inhalation solutions may also be formulated with a propellant for aerosol delivery. In yet another embodiment, solutions may be nebulized. Pulmonary administration is further described in Interntaional Pub. No. WO 94/20069, which describes the pulmonary delivery of chemically modified proteins.

It is also contemplated that certain formulations may be administered orally. In one embodiment of the present invention, HEH4 molecules or HEH4 selective bind agents that are administered in this fashion can be formulated with or without those carriers customarily used in the compounding of solid dosage forms such as tablets and capsules. For example, a capsule may be designed to release the active portion of the formulation at the point in the gastrointestinal tract when bioavailability is maximized and pre-systemic degradation is minimized. Additional agents can be included to facilitate absorption of the HEH4 molecule or HEH4 selective bind agent. Diluents, flavorings, low melting point waxes, vegetable oils, lubricants, suspending agents, tablet disintegrating agents, and binders may also be employed.

Another pharmaceutical composition may involve an effective quantity of HEH4 molecules or HEH4 selective bind agents in a mixture with non-toxic excipients that are suitable for the manufacture of tablets. By dissolving the tablets in sterile water, or another appropriate vehicle, solutions can be prepared in unit-dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional HEH4 molecule or HEH4 selective bind agent pharmaceutical compositions will be evident to those skilled in the art, including formulations involving HEH4 molecules or HEH4 selective bind agents in sustained- or controlled-delivery formulations. Techniques for formulating a variety of other sustained- or controlled-delivery means, such as liposome carriers, bio-erodible microparticles or porous beads and depot injections, are also known to those skilled in the art. See, e.g., International Pub. No. WO 93/15722, which describes the controlled release of porous polymeric microparticles for the delivery of pharmaceutical compositions.

Additional examples of sustained-release preparations include semipermeable polymer matrices in the form of shaped articles, e.g. films, or microcapsules. Sustained release matrices may include polyesters, hydrogels, polylactides (U.S. Pat. No. 3,773,919 and European Patent No. 058481), copolymers of L-glutamic acid and gamma ethyl-L-glutamate (Sidman et al., 1983, Biopolymers 22:547-56), poly(2-hydroxyethyl-methacrylate) (Langer et al., 1981, J. Biomed. Mater. Res. 15:167-277 and Langer, 1982, Chem. Tech. 12:98-105), ethylene vinyl acetate (Langer et al., supra) or poly-D(−)-3-hydroxybutyric acid (European Patent No. 133988). Sustained-release compositions may also include liposomes, which can be prepared by any of several methods known in the art. See, e.g., Eppstein et al., 1985, Proc. Natl. Acad. Sci. USA 82:3688-92; and European Patent Nos. 036676, 088046, and 143949.

The HEH4 molecule or HEH4 selective bind agent pharmaceutical composition to be used for in vivo administration typically must be sterile. This may be accomplished by filtration through sterile filtration membranes. Where the composition is lyophilized, sterilization using this method may be conducted either prior to, or following, lyophilization and reconstitution. The composition for parenteral administration may be stored in lyophilized form or in a solution. In addition, parenteral compositions generally are placed into a container having a sterile access port, for example, an intravenous solution bag or vial having a stopper pierceable by a hypodermic injection needle.

Once the pharmaceutical composition has been formulated, it may be stored in sterile vials as a solution, suspension, gel, emulsion, solid, or as a dehydrated or lyophilized powder. Such formulations may be stored either in a ready-to-use form or in a form (e.g., lyophilized) requiring reconstitution prior to administration.

In a specific embodiment, the present invention is directed to kits for producing a single-dose administration unit. The kits may each contain both a first container having a dried protein and a second container having an aqueous formulation. Also included within the scope of this invention are kits containing single and multi-chambered pre-filled syringes (e.g., liquid syringes and lyosyringes).

The effective amount of a HEH4 molecule or HEH4 selective bind agent pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which the HEH4 molecule or HEH4 selective bind agent is being used, the route of administration, and the size (body weight, body surface, or organ size) and condition (the age and general health) of the patient. Accordingly, the clinician may titer the dosage and modify the route of administration to obtain the optimal therapeutic effect. A typical dosage may range from about 0.1 μg/kg to up to about 100 mg/kg or more, depending on the factors mentioned above. In other embodiments, the dosage may range from 0.1 μg/kg up to about 100 mg/kg; or 1 μg/kg up to about 100 mg/kg; or 5 μg/kg up to about 100 mg/kg.

The frequency of dosing will depend upon the pharmacokinetic parameters of the HEH4 molecule or HEH4 selective bind agent in the formulation being used. Typically, a clinician will administer the composition until a dosage is reached that achieves the desired effect. The composition may therefore be administered as a single dose, as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages may be ascertained through use of appropriate dose-response data.

The route of administration of the pharmaceutical composition is in accord with known methods, e.g., orally; through injection by intravenous, intraperitoneal, intracerebral (intraparenchymal), intracerebroventricular, intramuscular, intraocular, intraarterial, intraportal, or intralesional routes; by sustained release systems; or by implantation devices. Where desired, the compositions may be administered by bolus injection or continuously by infusion, or by implantation device.

Alternatively or additionally, the composition may be administered locally via implantation of a membrane, sponge, or other appropriate material onto which the desired molecule has been absorbed or encapsulated. Where an implantation device is used, the device may be implanted into any suitable tissue or organ, and delivery of the desired molecule may be via diffusion, timed-release bolus, or continuous administration.

In some cases, it may be desirable to use HEH4 molecule or HEH4 selective bind agent pharmaceutical compositions in an ex vivo manner. In such instances, cells, tissues, or organs that have been removed from the patient are exposed to HEH4 molecule or HEH4 selective bind agent pharmaceutical compositions after which the cells, tissues, or organs are subsequently implanted back into the patient.

In other cases, a HEH4 polypeptide can be delivered by implanting certain cells that have been genetically engineered, using methods such as those described herein, to express and secrete the HEH4 polypeptide. Such cells may be animal or human cells, and may be autologous, heterologous, or xenogeneic. Optionally, the cells may be immortalized. In order to decrease the chance of an immunological response, the cells may be encapsulated to avoid infiltration of surrounding tissues. The encapsulation materials are typically biocompatible, semi-permeable polymeric enclosures or membranes that allow the release of the protein product(s) but prevent the destruction of the cells by the patient's immune system or by other detrimental factors from the surrounding tissues.

As discussed herein, it may be desirable to treat isolated cell populations (such as stem cells, lymphocytes, red blood cells, chondrocytes, neurons, and the like) with one or more HEH4 molecules or HEH4 selective bind agents. This can be accomplished by exposing the isolated cells to the polypeptide directly, where it is in a form that is permeable to the cell membrane.

Additional embodiments of the present invention relate to cells and methods (e.g., homologous recombination and/or other recombinant production methods) for both the in vitro production of therapeutic polypeptides and for the production and delivery of therapeutic polypeptides by gene therapy or cell therapy. Homologous and other recombination methods may be used to modify a cell that contains a normally transcriptionally-silent HEH4 gene, or an under-expressed gene, and thereby produce a cell that expresses therapeutically efficacious amounts of HEH4 polypeptides.

Homologous recombination is a technique originally developed for targeting genes to induce or correct mutations in transcriptionally active genes. Kucherlapati, 1989, Prog. in Nucl. Acid Res. & Mol. Biol. 36:301. The basic technique was developed as a method for introducing specific mutations into specific regions of the mammalian genome (Thomas et al., 1986, Cell 44:419-28; Thomas and Capecchi, 1987, Cell 51:503-12; Doetschman et al., 1988, Proc. Natl. Acad. Sci. U.S.A. 85:8583-87) or to correct specific mutations within defective genes (Doetschman et al., 1987, Nature 330:576-78). Exemplary homologous recombination techniques are described in U.S. Pat. No. 5,272,071; European Patent Nos. 9193051 and 505500; and International Pub. Nos. WO 91/09955 and WO 91/09955).

Through homologous recombination, the DNA sequence to be inserted into the genome can be directed to a specific region of the gene of interest by attaching it to targeting DNA. The targeting DNA is a nucleotide sequence that is complementary (homologous) to a region of the genomic DNA. Small pieces of targeting DNA that are complementary to a specific region of the genome are put in contact with the parental strand during the DNA replication process. It is a general property of DNA that has been inserted into a cell to hybridize, and therefore, recombine with other pieces of endogenous DNA through shared homologous regions. If this complementary strand is attached to an oligonucleotide that contains a mutation or a different sequence or an additional nucleotide, it too is incorporated into the newly synthesized strand as a result of the recombination. As a result of the proofreading function, it is possible for the new sequence of DNA to serve as the template. Thus, the transferred DNA is incorporated into the genome.

Attached to these pieces of targeting DNA are regions of DNA that may interact with or control the expression of a HEH4 polypeptide, e.g., flanking sequences. For example, a promoter/enhancer element, a suppressor, or an exogenous transcription modulatory element is inserted in the genome of the intended host cell in proximity and orientation sufficient to influence the transcription of DNA encoding the desired HEH4 polypeptide. The control element controls a portion of the DNA present in the host cell genome. Thus, the expression of the desired HEH4 polypeptide may be achieved not by transfection of DNA that encodes the HEH4 gene itself, but rather by the use of targeting DNA (containing regions of homology with the endogenous gene of interest) coupled with DNA regulatory segments that provide the endogenous gene sequence with recognizable signals for transcription of a HEH4 gene.

In an exemplary method, the expression of a desired targeted gene in a cell (i.e., a desired endogenous cellular gene) is altered via homologous recombination into the cellular genome at a preselected site, by the introduction of DNA that includes at least a regulatory sequence, an exon, and a splice donor site. These components are introduced into the chromosomal (genomic) DNA in such a manner that this, in effect, results in the production of a new transcription unit (in which the regulatory sequence, the exon, and the splice donor site present in the DNA construct are operatively linked to the endogenous gene). As a result of the introduction of these components into the chromosomal DNA, the expression of the desired endogenous gene is altered.

Altered gene expression, as described herein, encompasses activating (or causing to be expressed) a gene which is normally silent (unexpressed) in the cell as obtained, as well as increasing the expression of a gene which is not expressed at physiologically significant levels in the cell as obtained. The embodiments further encompass changing the pattern of regulation or induction such that it is different from the pattern of regulation or induction that occurs in the cell as obtained, and reducing (including eliminating) the expression of a gene which is expressed in the cell as obtained.

One method by which homologous recombination can be used to increase, or cause, HEH4 polypeptide production from a cell's endogenous HEH4 gene involves first using homologous recombination to place a recombination sequence from a site-specific recombination system (e.g., Cre/loxP, FLP/FRT) (Sauer, 1994, Curr. Opin. Biotechnol., 5:521-27; Sauer, 1993, Methods Enzymol., 225:890-900) upstream of (i.e., 5′ to) the cell's endogenous genomic HEH4 polypeptide coding region. A plasmid containing a recombination site homologous to the site that was placed just upstream of the genomic HEH4 polypeptide coding region is introduced into the modified cell line along with the appropriate recombinase enzyme. This recombinase causes the plasmid to integrate, via the plasmid's recombination site, into the recombination site located just upstream of the genomic HEH4 polypeptide coding region in the cell line (Baubonis and Sauer, 1993, Nucleic Acids Res. 21:2025-29; O'Gorman et al., 1991, Science 251:1351-55). Any flanking sequences known to increase transcription (e.g., enhancer/promoter, intron, translational enhancer), if properly positioned in this plasmid, would integrate in such a manner as to create a new or modified transcriptional unit resulting in de novo or increased HEH4 polypeptide production from the cell's endogenous HEH4 gene.

A further method to use the cell line in which the site-specific recombination sequence had been placed just upstream of the cell's endogenous genomic HEH4 polypeptide coding region is to use homologous recombination to introduce a second recombination site elsewhere in the cell line's genome. The appropriate recombinase enzyme is then introduced into the two-recombination-site cell line, causing a recombination event (deletion, inversion, and translocation) (Sauer, 1994, Curr. Opin. Biotechnol., 5:521-27; Sauer, 1993, Methods Enzymol., 225:890-900) that would create a new or modified transcriptional unit resulting in de novo or increased HEH4 polypeptide production from the cell's endogenous HEH4 gene.

An additional approach for increasing, or causing, the expression of HEH4 polypeptide from a cell's endogenous HEH4 gene involves increasing, or causing, the expression of a gene or genes (e.g., transcription factors) and/or decreasing the expression of a gene or genes (e.g., transcriptional repressors) in a manner which results in de novo or increased HEH4 polypeptide production from the cell's endogenous HEH4 gene. This method includes the introduction of a non-naturally occurring polypeptide (e.g., a polypeptide comprising a site specific DNA binding domain fused to a transcriptional factor domain) into the cell such that de novo or increased HEH4 polypeptide production from the cell's endogenous HEH4 gene results.

The present invention further relates to DNA constructs useful in the method of altering expression of a target gene. In certain embodiments, the exemplary DNA constructs comprise: (a) one or more targeting sequences, (b) a regulatory sequence, (c) an exon, and (d) an unpaired splice-donor site. The targeting sequence in the DNA construct directs the integration of elements (a)-(d) into a target gene in a cell such that the elements (b)-(d) are operatively linked to sequences of the endogenous target gene. In another embodiment, the DNA constructs comprise: (a) one or more targeting sequences, (b) a regulatory sequence, (c) an exon, (d) a splice-donor site, (e) an intron, and (f) a splice-acceptor site, wherein the targeting sequence directs the integration of elements (a)-(f) such that the elements of (b)-(f) are operatively linked to the endogenous gene. The targeting sequence is homologous to the preselected site in the cellular chromosomal DNA with which homologous recombination is to occur. In the construct, the exon is generally 3′ of the regulatory sequence and the splice-donor site is 3′ of the exon.

If the sequence of a particular gene is known, such as the nucleic acid sequence of HEH4 polypeptide presented herein, a piece of DNA that is complementary to a selected region of the gene can be synthesized or otherwise obtained, such as by appropriate restriction of the native DNA at specific recognition sites bounding the region of interest. This piece serves as a targeting sequence upon insertion into the cell and will hybridize to its homologous region within the genome. If this hybridization occurs during DNA replication, this piece of DNA, and any additional sequence attached thereto, will act as an Okazaki fragment and will be incorporated into the newly synthesized daughter strand of DNA. The present invention, therefore, includes nucleotides encoding a HEH4 polypeptide, which nucleotides may be used as targeting sequences.

HEH4 polypeptide cell therapy, e.g., the implantation of cells producing HEH4 polypeptides, is also contemplated. This embodiment involves implanting cells capable of synthesizing and secreting a biologically active form of HEH4 polypeptide. Such HEH4 polypeptide-producing cells can be cells that are natural producers of HEH4 polypeptides or may be recombinant cells whose ability to produce HEH4 polypeptides has been augrnented by transformation with a gene encoding the desired HEH4 polypeptide or with a gene augmenting the expression of HEH4 polypeptide. Such a modification may be accomplished by means of a vector suitable for delivering the gene as well as promoting its expression and secretion. In order to minimize a potential immunological reaction in patients being administered a HEH4 polypeptide, as may occur with the administration of a polypeptide of a foreign species, it is preferred that the natural cells producing HEH4 polypeptide be of human origin and produce human HEH4 polypeptide. Likewise, it is preferred that the recombinant cells producing HEH4 polypeptide be transformed with an expression vector containing a gene encoding a human HEH4 polypeptide.

Implanted cells may be encapsulated to avoid the infiltration of surrounding tissue. Human or non-human animal cells may be implanted in patients in biocompatible, semipermeable polymeric enclosures or membranes that allow the release of HEH4 polypeptide, but that prevent the destruction of the cells by the patient's immune system or by other detrimental factors from the surrounding tissue. Alternatively, the patient's own cells, transformed to produce HEH4 polypeptides ex vivo, may be implanted directly into the patient without such encapsulation.

Techniques for the encapsulation of living cells are known in the art, and the preparation of the encapsulated cells and their implantation in patients may be routinely accomplished. For example, Baetge et al. (International Pub. No. WO 95/05452 and International Pub. No. WO 95/05452) describe membrane capsules containing genetically engineered cells for the effective delivery of biologically active molecules. The capsules are biocompatible and are easily retrievable. The capsules encapsulate cells transfected with recombinant DNA molecules comprising DNA sequences coding for biologically active molecules operatively linked to promoters that are not subject to down-regulation in vivo upon implantation into a mammalian host. The devices provide for the delivery of the molecules from living cells to specific sites within a recipient. In addition, see U.S. Pat. Nos. 4,892,538; 5,011,472; and 5,106,627. A system for encapsulating living cells is described in International Pub. No. WO 91/10425 (Aebischer et al.). See also, International Pub. No. WO 91/10470 (Aebischer et al.); Winn et al., 1991, Exper. Neurol. 113:322-29; Aebischer et al., 1991, Exper. Neurol. 111:269-75; and Tresco et al., 1992, ASAIO 38:17-23.

In vivo and in vitro gene therapy delivery of HEH4 polypeptides is also envisioned. One example of a gene therapy technique is to use the HEH4 gene (either genomic DNA, cDNA, and/or synthetic DNA) encoding a HEH4 polypeptide that may be operably linked to a constitutive or inducible promoter to form a “gene therapy DNA construct.” The promoter may be homologous or heterologous to the endogenous HEH4 gene, provided that it is active in the cell or tissue type into which the construct will be inserted. Other components of the gene therapy DNA construct may optionally include DNA molecules designed for site-specific integration (e.g., endogenous sequences useful for homologous recombination), tissue-specific promoters, enhancers or silencers, DNA molecules capable of providing a selective advantage over the parent cell, DNA molecules useful as labels to identify transformed cells, negative selection systems, cell specific binding agents (as, for example, for cell targeting), cell-specific internalization factors, transcription factors enhancing expression from a vector, and factors enabling vector production.

A gene therapy DNA construct can then be introduced into cells (either ex vivo or in vivo) using viral or non-viral vectors. One means for introducing the gene therapy DNA construct is by means of viral vectors as described herein. Certain vectors, such as retroviral vectors, will deliver the DNA construct to the chromosomal DNA of the cells, and the gene can integrate into the chromosomal DNA. Other vectors will function as episomes, and the gene therapy DNA construct will remain in the cytoplasm.

In yet other embodiments, regulatory elements can be included for the controlled expression of the HEH4 gene in the target cell. Such elements are turned on in response to an appropriate effector. In this way, a therapeutic polypeptide can be expressed when desired. One conventional control means involves the use of small molecule dimerizers or rapalogs to dimerize chimeric proteins which contain a small molecule-binding domain and a domain capable of initiating a biological process, such as a DNA-binding protein or transcriptional activation protein (see International Pub. Nos. WO 96/41865, WO 97/31898, and WO 97/31899). The dimerization of the proteins can be used to initiate transcription of the transgene.

An alternative regulation technology uses a method of storing proteins expressed from the gene of interest inside the cell as an aggregate or cluster. The gene of interest is expressed as a fusion protein that includes a conditional aggregation domain that results in the retention of the aggregated protein in the endoplasmic reticulum. The stored proteins are stable and inactive inside the cell. The proteins can be released, however, by administering a drug (e.g., small molecule ligand) that removes the conditional aggregation domain and thereby specifically breaks apart the aggregates or clusters so that the proteins may be secreted from the cell. See Aridor et al., 2000, Science 287:816-17 and Rivera et al., 2000, Science 287:826-30.

Other suitable control means or gene switches include, but are not limited to, the systems described herein. Mifepristone (RU486) is used as a progesterone antagonist. The binding of a modified progesterone receptor ligand-binding domain to the progesterone antagonist activates transcription by forming a dimer of two transcription factors that then pass into the nucleus to bind DNA. The ligand-binding domain is modified to eliminate the ability of the receptor to bind to the natural ligand. The modified steroid hormone receptor system is further described in U.S. Pat. No. 5,364,791 and International Pub. Nos. WO 96/40911 and WO 97/10337.

Yet another control system uses ecdysone, a fruit fly steroid hormone that binds to and activates an ecdysone receptor (cytoplasmic receptor). The receptor then translocates to the nucleus to bind a specific DNA response element (promoter from ecdysone-responsive gene). The ecdysone receptor includes a transactivation domain, DNA-binding domain, and ligand-binding domain to initiate transcription. The ecdysone system is further described in U.S. Pat. No. 5,514,578 and International Pub. Nos. WO 97/38117, WO 96/37609, and WO 93/03162.

Another control means uses a positive tetracycline-controllable transactivator. This system involves a mutated tet repressor protein DNA-binding domain (mutated tet R-4 amino acid changes which resulted in a reverse tetracycline-regulated transactivator protein, i.e., it binds to a tet operator in the presence of tetracycline) linked to a polypeptide that activates transcription. Such systems are described in U.S. Pat. Nos. 5,464,758, 5,650,298, and 5,654,168.

Additional expression control systems and nucleic acid constructs are described in U.S. Pat. Nos. 5,741,679 and 5,834,186, to Innovir Laboratories Inc.

In vivo gene therapy may be accomplished by introducing the gene encoding HEH4 polypeptide into cells via local injection of a HEH4 nucleic acid molecule or by other appropriate viral or non-viral delivery vectors. Hefti 1994, Neurobiology 25:1418-35. For example, a nucleic acid molecule encoding a HEH4 polypeptide may be contained in an adeno-associated virus (AAV) vector for delivery to the targeted cells (see, e.g., International Pub. Nos. WO 95/34670 and WO 95/34670). The recombinant AAV genome typically contains AAV inverted terminal repeats flanking a DNA sequence encoding a HEH4 polypeptide operably linked to functional promoter and polyadenylation sequences.

Alternative suitable viral vectors include, but are not limited to, retrovirus, adenovirus, herpes simplex virus, lentivirus, hepatitis virus, parvovirus, papovavirus, poxvirus, alphavirus, coronavirus, rhabdovirus, paramyxovirus, and papilloma virus vectors. U.S. Pat. No. 5,672,344 describes an in vivo viral-mediated gene transfer system involving a recombinant neurotrophic HSV-1 vector. U.S. Pat. No. 5,399,346 provides examples of a process for providing a patient with a therapeutic protein by the delivery of human cells that have been treated in vitro to insert a DNA segment encoding a therapeutic protein. Additional methods and materials for the practice of gene therapy techniques are described in U.S. Pat. No. 5,631,236 (involving adenoviral vectors), 5,672,510 (involving retroviral vectors), U.S. Pat. No. 5,635,399 (involving retroviral vectors expressing cytokines).

Nonviral delivery methods include, but are not limited to, liposome-mediated transfer, naked DNA delivery (direct injection), receptor-mediated transfer (ligand-DNA complex), electroporation, calcium phosphate precipitation, and microparticle bombardment (e.g., gene gun). Gene therapy materials and methods may also include inducible promoters, tissue-specific enhancer-promoters, DNA sequences designed for site-specific integration, DNA sequences capable of providing a selective advantage over the parent cell, labels to identify transformed cells, negative selection systems and expression control systems (safety measures), cell-specific binding agents (for cell targeting), cell-specific internalization factors, and transcription factors to enhance expression by a vector as well as methods of vector manufacture. Such additional methods and materials for the practice of gene therapy techniques are described in U.S. Pat. No. 4,970,154 (involving electroporation techniques), U.S. Pat. No. 5,679,559 (describing a lipoprotein-containing system for gene delivery), U.S. Pat. No. 5,676,954 (involving liposome carriers), U.S. Pat. No. 5,593,875 (describing methods for calcium phosphate transfection), and U.S. Pat. No. 4,945,050 (describing a process wherein biologically active particles are propelled at cells at a speed whereby the particles penetrate the surface of the cells and become incorporated into the interior of the cells), and International Pub. No. WO 96/40958 (involving nuclear ligands).

It is also contemplated that HEH4 gene therapy or cell therapy can further include the delivery of one or more additional polypeptide(s) in the same or a different cell(s). Such cells may be separately introduced into the patient, or the cells may be contained in a single implantable device, such as the encapsulating membrane described above, or the cells may be separately modified by means of viral vectors.

A means to increase endogenous HEH4 polypeptide expression in a cell via gene therapy is to insert one or more enhancer elements into the HEH4 polypeptide promoter, where the enhancer elements can serve to increase transcriptional activity of the HEH4 gene. The enhancer elements used will be selected based on the tissue in which one desires to activate the gene—enhancer elements known to confer promoter activation in that tissue will be selected. For example, if a gene encoding a HEH4 polypeptide is to be “turned on” in T-cells, the ick promoter enhancer element may be used. Here, the functional portion of the transcriptional element to be added may be inserted into a fragment of DNA containing the HEH4 polypeptide promoter (and optionally, inserted into a vector and/or 5′ and/or 3′ flanking sequences) using standard cloning techniques. This construct, known as a “homologous recombination construct,” can then be introduced into the desired cells either ex vivo or in vivo.

Gene therapy also can be used to decrease HEH4 polypeptide expression by modifying the nucleotide sequence of the endogenous promoter. Such modification is typically accomplished via homologous recombination methods. For example, a DNA molecule containing all or a portion of the promoter of the HEH4 gene selected for inactivation can be engineered to remove and/or replace pieces of the promoter that regulate transcription. For example, the TATA box and/or the binding site of a transcriptional activator of the promoter may be deleted using standard molecular biology techniques; such deletion can inhibit promoter activity thereby repressing the transcription of the corresponding HEH4 gene. The deletion of the TATA box or the transcription activator binding site in the promoter may be accomplished by generating a DNA construct comprising all or the relevant portion of the HEH4 polypeptide promoter (from the same or a related species as the HEH4 gene to be regulated) in which one or more of the TATA box and/or transcriptional activator binding site nucleotides are mutated via substitution, deletion and/or insertion of one or more nucleotides. As a result, the TATA box and/or activator binding site has decreased activity or is rendered completely inactive. This construct, which also will typically contain at least about 500 bases of DNA that correspond to the native (endogenous) 5′ and 3′ DNA sequences adjacent to the promoter segment that has been modified, may be introduced into the appropriate cells (either ex vivo or in vivo) either directly or via a viral vector as described herein. Typically, the integration of the construct into the genomic DNA of the cells will be via homologous recombination, where the 5′ and 3′ DNA sequences in the promoter construct can serve to help integrate the modified promoter region via hybridization to the endogenous chromosomal DNA.

Therapeutic Uses

HEH4 nucleic acid molecules, polypeptides, and agonists and antagonists thereof can be used to treat, diagnose, ameliorate, or prevent a number of diseases, disorders, or conditions, including those recited herein. Examples of such antagonists include, but are not limited to, antibodies and peptibodies (as described in International Publication No. WO 00/24782).

HEH4 polypeptide agonists and antagonists include those molecules which regulate HEH4 polypeptide activity and either increase or decrease at least one activity of the mature form of the HEH4 polypeptide. Agonists or antagonists may be co-factors, such as a protein, peptide, carbohydrate, lipid, or small molecular weight molecule, which interact with HEH4 polypeptide and thereby regulate its activity. Potential polypeptide agonists or antagonists include antibodies that react with either soluble or cell-bound forms of HEH4 polypeptides. Molecules that regulate HEH4 polypeptide expression typically include nucleic acids encoding HEH4 polypeptide that can act as anti-sense regulators of expression.

Bone tissue consists of a matrix of collagenous and noncollagenous proteins, minerals (largely calcium and phosphorous), and cells. Three types of cells are involved in the dynamic process by which bone is continually formed and resorbed: osteocytes, osteoblasts, and osteoclasts. Osteoblasts promote formation of bone tissue whereas osteoclasts are associated with resorption. Resorption, or the dissolution of bone matrix and mineral, is a fast and efficient process compared to bone formation and can release large amounts of mineral from bone. Osteoclasts are involved in the regulation of the normal remodeling of skeletal tissue and in resorption induced by hormones. For instance, resorption is stimulated by the secretion of parathyroid hormone in response to decreasing concentrations of calcium ion in extracellular fluids. In contrast, inhibition of resorption is the principal function of calcitonin. In addition, metabolites of vitamin D alter the responsiveness of bone to parathyroid hormone and calcitonin.

Following skeletal maturity, the amount of bone in the skeleton reflects the balance (or imbalance) of bone formation and bone resorption. Peak bone mass occurs after skeletal maturity prior to the fourth decade. Between the fourth and fifth decades, the equilibrium shifts and bone resorption dominates. The inevitable decrease in bone mass with advancing years starts earlier in females than males and is distinctly accelerated after menopause in some females (principally those of Caucasian and Asian descent).

Osteopenia is a condition relating generally to any decrease in bone mass to below normal levels. Such a condition may arise from a decrease in the rate of bone synthesis or an increase in the rate of bone destruction or both. The most common form of osteopenia is primary osteoporosis, also referred to as postmenopausal and senile osteoporosis. This form of osteoporosis is a consequence of the universal loss of bone with age and is usually a result of increase in bone resorption with a normal rate of bone formation. About 25 to 30 percent of all white females in the United States develop symptomatic osteoporosis. A direct relationship exists between osteoporosis and the incidence of hip, femoral, neck, and inter-trochanteric fracture in women 45 years and older. Elderly males develop symptomatic osteoporosis between the ages of 50 and 70, but the disease primarily affects females.

The cause of postmenopausal and senile osteoporosis is unknown. Several factors have been identified which may contribute to the condition. They include alteration in hormone levels accompanying aging, and inadequate calcium consumption attributed to decreased intestinal absorption of calcium and other minerals. Treatments have usually included hormone therapy, dietary supplements, and anti-resorptive agents in an attempt to retard the process. More effective therapies are desirable.

Molecules that can decrease HEH4 levels or activity such as certain HEH4 nucleic acid molecules (e.g., anti-sense nucleic acids, interference RNA), dominant negative HEH4 polypeptides, and antagonists of HEH4 polypeptides of the present invention may be used to treat, ameliorate, or prevent diseases or disorders characterized by a net bone loss (such as osteopenia, osteoporosis, or osteolysis) or below normal bone strength. For example, such molecules may be used to stimulate the rate of bone formation. In this manner, an individual may be treated with molecules that can decrease HEH4 levels or activity in order to stimulate the rate of bone formation where the formation rate is below normal, or where the bone resorption rate is excessive, in order to compensate for below normal bone mass or bone strength.

Conditions that may be treatable with molecules that can decrease HEH4 levels or activity (such as certain HEH4 nucleic acid molecules, dominant negative HEH4 polypeptides, and antagonists of HEH4 polypeptides) of the present invention include the following: osteoporosis, such as primary osteoporosis, endocrine osteoporosis (hyperthyroidism, hyperparathyroidism, Cushing's syndrome, and acromegaly), hereditary and congenital forms of osteoporosis (osteogenesis imperfecta, homocystinuria, Menkes' syndrome, and Riley-Day syndrome), and osteoporosis due to immobilization of extremities; Paget's disease of bone (osteitis deformans) in adults and juveniles; osteomyelitis, or an infectious lesion in bone, leading to bone loss; hypercalcemia resulting from solid tumors (breast, lung, and kidney) and hematologic malignancies (multiple myeloma, lymphoma, and leukemia), idiopathic hypercalcemia, and hypercalcemia associated with hyperthyroidism and renal function disorders; osteopenia following surgery, induced by steroid administration, and associated with disorders of the small and large intestine and with chronic hepatic and renal diseases; osteonecrosis, or bone cell death, associated with traumatic injury or nontraumatic necrosis associated with Gaucher's disease, sickle cell anemia, systemic lupus erythematosus, rheumatoid arthritis, periodontal disease, osteolytic metastasis, and other conditions. Other low bone mass or low bone strength diseases and disorders are encompassed within the scope of the invention.

Diseases or disorders characterized by excessive local or systemic bone mass or bone strength may be treatable with HEH4 nucleic acid molecules, polypeptides, and agonists thereof.

Based on the role of HEH4 in bone biology, as described herein, the likely role of bone in the biology of cartilage-related diseases, as well as the expression of HEH4 in elderly human articular cartilage chondrocytes, the HEH4 nucleic acid molecules, polypeptides, and agonists and antagonists of the present invention may be used to treat, ameliorate, or prevent cartilage-related diseases and disorders such as osteoarthritis and rheumatoid arthritis. Other cartilage-related diseases and disorders are encompassed within the scope of the invention.

HEH4 nucleic acid molecules, polypeptides, and agonists and antagonists of HEH4 polypeptide function may be used (simultaneously or sequentially) in combination with one or more cytokines, growth factors, antibiotics, anti-inflammatories, or chemotherapeutic agents as is appropriate for the condition being treated.

Other diseases or disorders caused by or mediated by undesirable levels of HEH4 polypeptides are encompassed within the scope of the invention. Undesirable levels include excessive levels of HEH4 polypeptides and sub-normal levels of HEH4 polypeptides.

Uses of HEH4 Nucleic Acids and Polypeptides

HEH4 nucleic acid molecules (including those that do not themselves encode biologically active polypeptides) may be useful as hybridization probes in diagnostic assays to test, either qualitatively or quantitatively, for the presence of a HEH4 nucleic acid molecule in mammalian tissue or bodily fluid samples.

Other methods may also be employed where it is desirable to inhibit the activity of one or more HEH4 polypeptides. Such inhibition may be effected by nucleic acid molecules that are complementary to and hybridize to expression control sequences (triple helix formation) or to HEH4 mRNA. For example, antisense DNA or RNA molecules, which have a sequence that is complementary to at least a portion of a HEH4 gene can be introduced into the cell. Anti-sense probes may be designed by available techniques using the sequence of the HEH4 gene disclosed herein. Typically, each such antisense molecule will be complementary to the start site (5′ end) of each selected HEH4 gene. When the antisense molecule then hybridizes to the corresponding HEH4 mRNA, translation of this mRNA is prevented or reduced. Anti-sense inhibitors provide information relating to the decrease or absence of a HEH4 polypeptide in a cell or organism.

Alternatively, gene therapy may be employed to create a dominant-negative inhibitor of one or more HEH4 polypeptides. In this situation, the DNA encoding a mutant polypeptide of each selected HEH4 polypeptide can be prepared and introduced into the cells of a patient using either viral or non-viral methods as described herein. Each such mutant is typically designed to compete with endogenous polypeptide in its biological role.

In addition, a HEH4 polypeptide, whether biologically active or not, may be used as an immunogen, that is, the polypeptide contains at least one epitope to which antibodies may be raised. Selective binding agents that bind to a HEH4 polypeptide (as described herein) may be used for in vivo and in vitro diagnostic purposes, including, but not limited to, use in labeled form to detect the presence of HEH4 polypeptide in a body fluid or cell sample. The antibodies may also be used to prevent, treat, or diagnose a number of diseases and disorders, including those recited herein. The antibodies may bind to a HEH4 polypeptide so as to diminish or block at least one activity characteristic of a HEH4 polypeptide, or may bind to a polypeptide to increase at least one activity characteristic of a HEH4 polypeptide (including by increasing the pharmacokinetics of the HEH4 polypeptide).

The HEH4 polypeptides of the present invention may be usful for cloning HEH4 polypeptide receptors, using an expression cloning strategy. Radiolabeled (¹²⁵Iodine) HEH4 polypeptide or affinity or activity-tagged HEH4 polypeptide (such as an Fc fusion or an alkaline phosphatase fusion) can be used in binding assays to identify a cell type or cell line or tissue that expresses HEH4 polypeptide receptors. RNA isolated from such cells or tissues can be converted to cDNA, cloned into a mammalian expression vector, and transfected into mammalian cells (such as COS or 293 cells) to create an expression library. A radiolabeled or tagged HEH4 polypeptide can then be used as an affinity ligand to identify and isolate from this library the subset of cells that express the HEH4 polypeptide receptors on their surface. DNA can then be isolated from these cells and transfected into mammalian cells to create a secondary expression library in which the fraction of cells expressing HEH4 polypeptide receptors is many-fold higher than in the original library. This enrichment process can be repeated iteratively until a single recombinant clone containing a HEH4 polypeptide receptor is isolated. Isolation of the HEH4 polypeptide receptors is useful for identifying or developing novel agonists and antagonists of the HEH4 polypeptide signaling pathway. Such agonists and antagonists include soluble HEH4 polypeptide receptors, anti-HEH4 polypeptide receptor antibodies, small molecules, or antisense oligonucleotides, and they may be used for treating, preventing, or diagnosing one or more of the diseases or disorders described herein.

HEH4 polypeptides may also be useful for cloning HEH4 ligands using an “expression cloning” strategy. Radiolabeled (¹²⁵Iodine) HEH4 polypeptide or “affinity/activity-tagged” HEH4 polypeptide (such as an Fc fusion or an alkaline phosphatase fusion) can be used in binding assays to identify a cell type, cell line, or tissue that expresses a HEH4 ligand. RNA isolated from such cells or tissues can then be converted to cDNA, cloned into a mammalian expression vector, and transfected into mammalian cells (e.g., COS or 293) to create an expression library. Radiolabeled or tagged HEH4 polypeptide can then be used as an affinity reagent to identify and isolate the subset of cells in this library expressing a HEH4 ligand. DNA is then isolated from these cells and transfected into mammalian cells to create a secondary expression library in which the fraction of cells expressing the HEH4 ligand would be many-fold higher than in the original library. This enrichment process can be repeated iteratively until a single recombinant clone containing the HEH4 ligand is isolated. Isolation of HEH4 ligands is useful for identifying or developing novel agonists and antagonists of the HEH4 signaling pathway. Such agonists and antagonists include HEH4 ligands, anti-HEH4 ligand antibodies, small molecules or antisense oligonucleotides.

The following examples are intended for illustration purposes only, and should not be construed as limiting the scope of the invention in any way.

### EXAMPLE 1

### Identification of Bone Targets by Expression Profiling

Homozygous OPG knockout mice are severely osteoporotic, exhibiting a marked reduction in trabecular bone tissue and extremely porous cortical bones. The bones of OPG knockout mice are very fragile and many of these mice exhibit bone fractures during their first two months of life. OPG knockout mice show a marked elevation in their osteoclast and osteoblast numbers and increased levels of bone remodeling. In addition, these mice exhibit a four- to five-fold increase in serum alkaline phosphatase, which further indicates that osteoblast-mediated bone synthesis in OPG knockout mice is increased. In contrast with OPG knockout mice, OPG transgenic mice are severely osteopetrotic. The tibias and other long bones of OPG transgenic mice lack osteoclasts and therefore, have a higher bone density, and their marrow cavities are filled with trabecular bone and cartilage. In addition, genes that are highly expressed in the femurs of OPG transgenic mice are not likely to be expressed in osteoclasts, as these bones are markedly depleted of functional bone resorbing cells. Furthermore, since osteoblast numbers are unchanged in OPG transgenic mouse bone, OPG transgenic bones can be used to identify genes that are expressed in osteoblasts, but not osteoclasts.

To identify genes involved in bone biology, expression profiling was performed using RNA collected from the crushed femurs and tibias of OPG knockout and normal mice. One of the sequences identified in the expression profiling analysis encodes HEH4, a protein having a predicted transmembrane domain and two extracellular Ig regions.

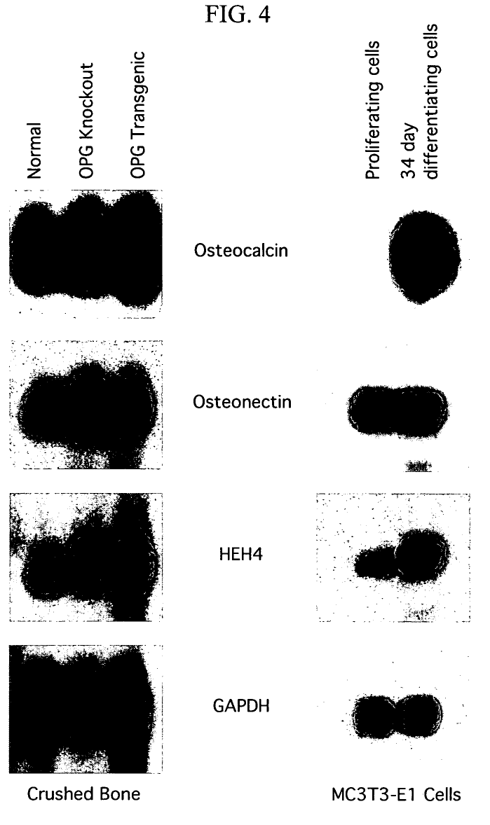

The results obtained by expression profiling were verified by Northern blot analysis using using a portion of murine HEH4 cDNA as a probe. A higher level of HEH4 mRNA expression was detected in crushed bone samples obtained from OPG knockout mice than in crushed bone samples obtained from normal mice and a higher level of HEH4 mRNA expression was detected in crushed bone samples obtained from OPG transgenic mouse than in crushed bone samples obtained from either OPG knockout or normal mice (FIG. 4). In addition, an increase in HEH4 mRNA expression was detected in differentiated cells of the murine osteogenic cell line MC3T3-E1 (FIG. 4). MC3T3-E1 cells, which are derived from calvarial bone, are committed to the osteoblast lineage and proliferate in culture without showing signs of osteoblast marker expression. Following growth in Vitamin C and B-glycerophosphate, MC3T3-E1 cells undergo differentiation, resulting in the upregulation of activated osteoblast markers such as alkaline phosphatase and osteocalcin, and the production of bone mineral matrix as detected by Von Kossa staining.

### EXAMPLE 2

### In Situ Localization of HEH4

The expression of HEH4 mRNA was localized by in situ hybridization analysis of normal embryonic and adult mouse tissues, bone tissue from a number of in vivo mouse models, and human knee samples from normal elderly humans as well as elderly humans afflicted with osteoarthritis, rheumatoid arthritis, or osteoporosis. Among the mouse models tested were OPG transgenic mice, OPG knockout mice, OPGL-treated mice, and tumor bearing mice. Normal mouse embryos at E10.5, 11.5, 13.5, 15.5 and 18.5 were also analyzed by in situ hybridization. The expression of HEH4 mRNA localized by in situ hybridization was consistent with the results obtained by Northern analysis.

Tissue samples were immersion fixed, embedded in paraffin, and sectioned at 5 μm. In situ hybridization was performed using standard techniques. Sectioned tissues were hybridized overnight at 60° C. in hybridization solution containing a ³³P-labeled antisense riboprobe that was complementary to the mouse HEH4 gene. The riboprobe was obtained by in vitro transcription of a clone containing a 1 kb insert of the mouse HEH4 cDNA sequence using standard techniques.

Following hybridization, sections were treated with RNaseA to digest unhybridized probe and then were rinsed in a series of SSC washes; the highest stringency wash comprising 0.1×SSC at 55° C. for 30 minutes. Sections were then dipped in emulsion, exposed for 3 weeks at 4° C., developed, and counterstained with hematoxylin and eosin. Tissue morphology and hybridization signal were simultaneously analyzed by darkfield and standard illumination for the following normal adult mouse tissues: nervous system (brain and peripheral nervous system), gastrointestinal system (parotid, submandibular, and sublingual glands; esophagus; stomach; duodenum; jejunum; ileum; proximal and distal colon; liver; and pancreas), cardiopulmonary system (heart, lung, trachea, and blood vessels); hematolymphoid system (lymph nodes, spleen, thymus, and bone marrow), urinary system (kidney and bladder), endocrine system (thyroid and adrenal glands), reproductive system (testis, prostrate, and epididymus, ovary, uterus, oviduct, mammary tissue, and placenta); and musculoskeletal system (bone, skeletal muscle, skin, and adipose tissue). For the in vivo mouse models, tissue morphology and hybridization signal were analyzed for bone.

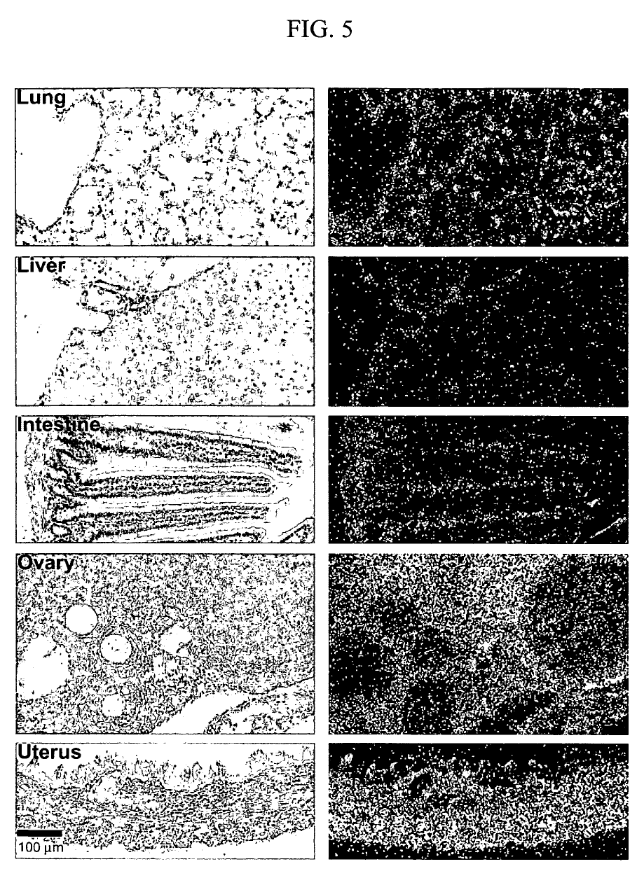

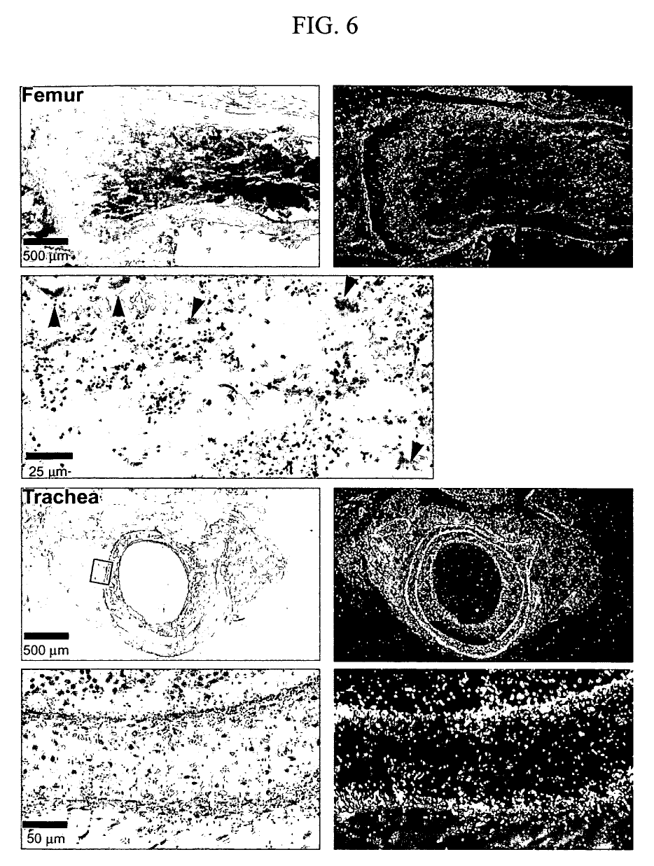

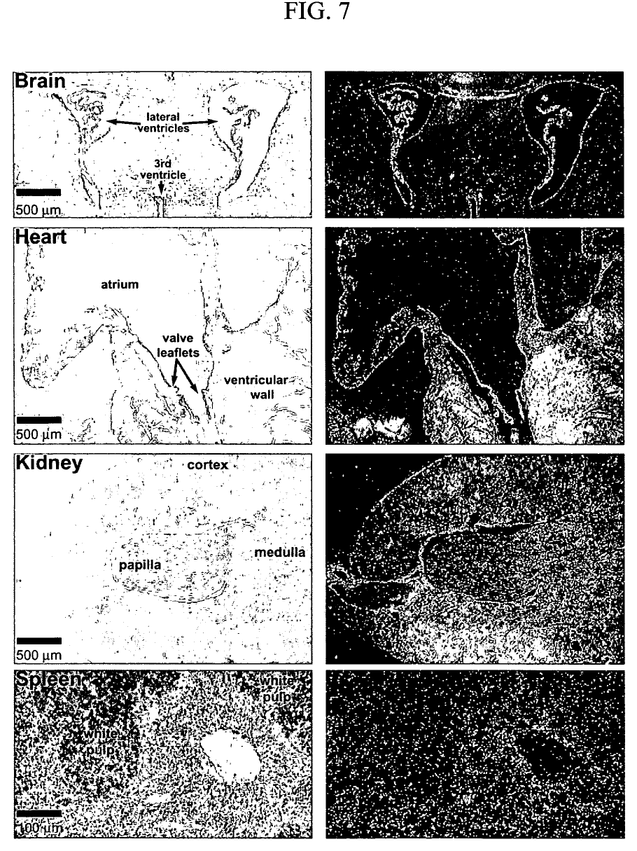

In the normal adult tissue, the highest level of HEH4 mRNA expression was detected in the alveolar cells of the lungs (FIG. 5); in the periosteal cells of bone and perichondral cells surrounding the hyaline cartilage of the trachea (FIG. 6); in connective tissue elements of the esophagus, stomach, ducts around the salivary glands, ovarian capsule, ovarian stroma, and ovarian ligaments (FIG. 5); and in valve leaflets of the heart (FIG. 7). Strong expression was also detected in spermatocytes of the testis, myometrium of the uterus (FIG. 5), and the placental chorionic plate and yolk sac. Moderate expression was found in brain ependymal cells lining the ventricles, in meningeal cells and in the choroid plexus primarily over the epithethelial component (FIG. 7). In the kidney, moderate expression was observed in the connective tissue of the capsule; lower, scattered expression was detected in the vascular elements of the cortex; and expression was concentrated in the medulla although the vascular association was less clear than in the cortex where the blood vessels are more widely scattered amongst the tubules (FIG. 7). There was also moderate expression in the muscular wall of the bladder. Low variable expression was discernible in lamina propria, submucosa, and muscularis cells of the duodenum, jejunum, and ileum (FIG. 7), as well as the proximal and distal colon. In the liver, low to moderate expression was found in cells associated with some vessels, especially the smooth muscle of the larger vessels (FIG. 5). Although expression was detected in the capsule surrounding the pancreas, there was little expression in cells associated with the larger pancreatic ducts. Some low and variable expression was detected in the subcapsular sinus area, but an even lower expression was observed in the lymph nodules. In the spleen, the expression was stronger over the red pulp areas, even though there was diffuse expression observed overall (FIG. 7). There was moderate expression in the muscular wall of the oviduct, as well some expression in the fibroblast and connective tissue-type cells of the mammary tissue.

The mouse embryonic tissue displayed a widespread mesenchymal pattern of HEH4 expression that remained approximately the same throughout development and was comparable to the adult pattern. The most striking embryonic HEH4 mRNA expression was in the perichondral cells surrounding the cartilage models of the developing bones. Expression in the liver (except for the capsule, which is strongly positive) was low or nondetectable at all ages examined (E10.5 to E18.5). In the lung, expression was confined to the mesenchymal cells with little or no signal in the epithelial cells. Likewise, expression was lacking in the epithelial cells of the intestinal mucosa, but present in lamina propria and serosa. Other areas where little to no signal was detected included the kidney, pancreas, brain (with the exception of the ventricular lining cells), and other neural tissues including trigeminal ganglion, DRG, retina, and cochlea.

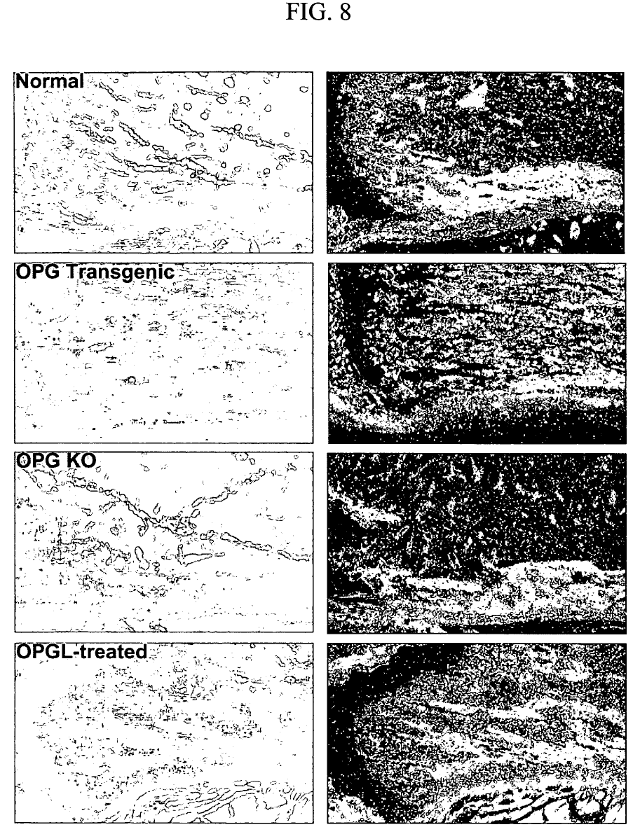

Expression of HEH4 mRNA in the various in vivo mouse models appeared to be closely associated with the level of osteoblast activation. Although osteoclasts were difficult to discern in the lightly counterstained sections examined, HEH4 mRNA expression was not detected in osteoclasts of the in vivo models examined. The pattern seen in normal bone, where signal is strongest in perichondral cells and lower in osteoblasts, was also seen in the OPG transgenic mouse, OPG knockout, OPGL-treated bone, and tumor bearing mouse. However, in the OPG knockout mouse and OPGL-treated mouse, a moderate level of overall expression was detected in the osteoblasts (FIG. 8). In the OPGL-treated mouse, most of the signal is confined to the region adjacent to the growth plate, whereas in the OPG knockout mouse, the expression is found in the lacunae and trabeculae throughout the length of the bones.

In situ hybridization was used to confirm the mRNA expression of HEH4 in human arthritic bone samples. Human knee tissue samples were obtained from individuals that were normal (two 55-58 year old females) or who had rheumatoid arthritis (two 73-80 year old females), degenerative joint disease (one 48 year old female and one 57 year old male), osteoarthritic/degenerative joint disease (two 73-74 year old 25 males), or osteoarthritis (three 68-78 year old females and one 71 year old male). In addition, assorted bone tissue samples were obtained from an osteoporotic individual (76 year old female). As a control, long bone, knee joint, femur head, lung, and white adipose tissues were obtained from a Cynomolgus monkey (3.5-4 years old).

Tissue samples were immersion fixed, embedded in paraffin, and sectioned at 5 μm. In situ hybridization was performed using standard techniques. Sectioned tissues were hybridized overnight at 60° C. in hybridization solution containing a ³³P-labeled antisense riboprobe that was complementary to the human HEH4 gene. The riboprobe was obtained by in vitro transcription of a clone containing a 900 bp insert from the middle of the human HEH4 coding region (and including the transmembrane domain) using standard techniques.

Following hybridization, sections were treated with RNaseA to digest unhybridized probe and then were rinsed in a series of SSC washes; the highest stringency wash comprising 0.1× SSC at 55° C. for 30 minutes. Sections were then dipped in emulsion, exposed for 3 weeks at 4° C., developed, and counterstained with hematoxylin and eosin. The samples were simultaneously analyzed by darkfield and standard illumination, with the relative levels of HEH4 expression shown in Table III.

TABLE III

| Arthritic | |
|---|---|
| Human | |
| Knee | |
| Tissue | |
| Sample | Relative Expression of HEH4 |
| OA | no expression |
| OA/DJD | weak expression in osteoblasts and bone lining cells |
| RA | moderate to strong variable expression in osteoblasts |
| | and bone lining cells; low expression in chondrocytes |
| OA/DJD | weak expression in osteoblasts and bone lining cells; |
| | weak diffuse expression in fibrous connective tissue |
| OA | questionable expression just above background in few |
| | osteoblasts or bone lining cells |
| OA | weak expression in 3 or 4 patches of osteoblasts or |
| | bone lining cells; weak expression in few chondrocytes |
| OA | questionable expression just above background in few |
| | osteoblasts or bone lining cells; |
| | low expression in chondrocytes |
| DJD | strong expression in osteoblast and bone lining cells; |
| | low to moderate variable expression in some chondrocytes |
| RA | low expression in pateches of osteoblasts and bone lining |
| | cells; low expression in chondrocytes |
| Normal | low expression in fibrous connective tissue; questionable |
| | expression just above background in osteoblasts or |
| | bone lining cells in femur; no expression in vertebrae |
| DJD | weak expression in small pateches of osteoblasts and |
| | bone lining cells |
| Normal | questionable expression just above background in |
| | osteoblasts or bone lining cells |
| OP | weak expression in small patches of osteoblasts or bone |
| | lining cells in vertebrae; no signal in femur |
| Monkey | |
| Bone | |
| Tissue | |
| Long bone | no expression |
| Knee joint | no expression |
| Femur head | no expression |
| Lung | very weak diffuse expression |
| White | very weak diffuse expression |
| adipose | |
| tissue | |

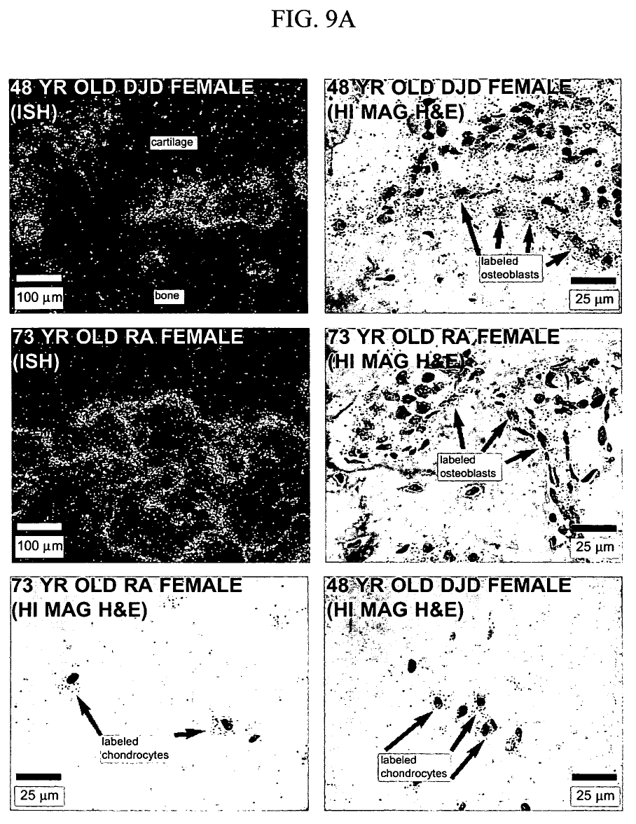

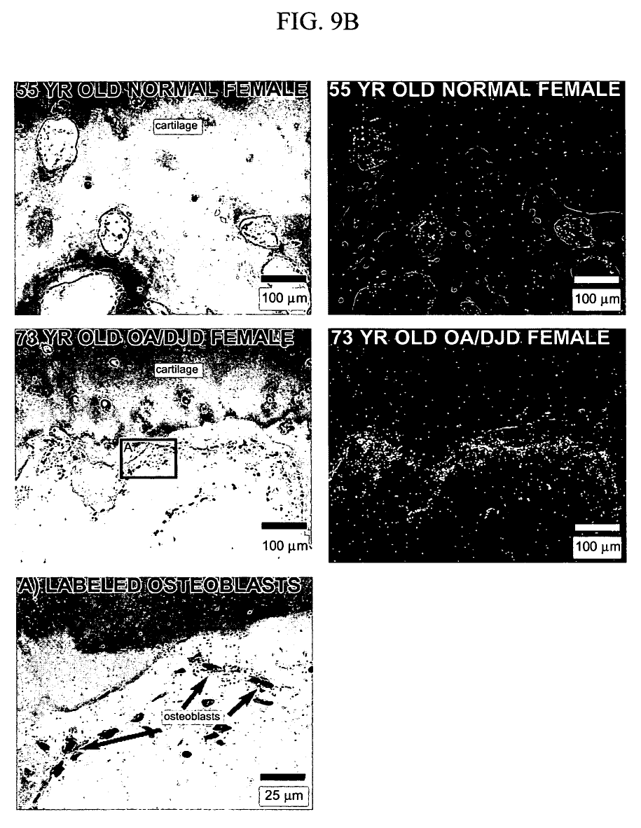

HEH4 expression was detected in the osteoblasts and bone lining cells of human knee samples from all of the individuals with rheumatoid arthritis, osteoporosis, or degenerative joint disease (FIGS. 9A-9B). Only one of four individuals with osteoarthritis, and none of the normal individuals showed HEH4 osteoblast labeling. Low expression of HEH4 was also detected in the articular cartilage of several inidviduals with osteoarthritis, rheumatoid arthritis, or degenerative joint disease. Although none of the monkey bone sections exhibited HEH4 mRNA expression in these experiments, this result may have been due to poor cross-species hybridization rather than a lack of HEH4 expression.

### EXAMPLE 3

### Overexpression of Murine HEH4 in Transgenic Mice

To assess the in vivo biological effect of increasing HEH4 levels or activity in bone, a plasmid vector was constructed that encodes mouse HEH4 under the control of the rat collagen 1a1 (3.6 kb) promoter, which is expressed in cells of the osteoblast lineage. Transgenic mice were generated using this DNA construct. HEH4 mRNA expression levels in femur samples obtained from transgenic mice were determined by northern blot analysis.

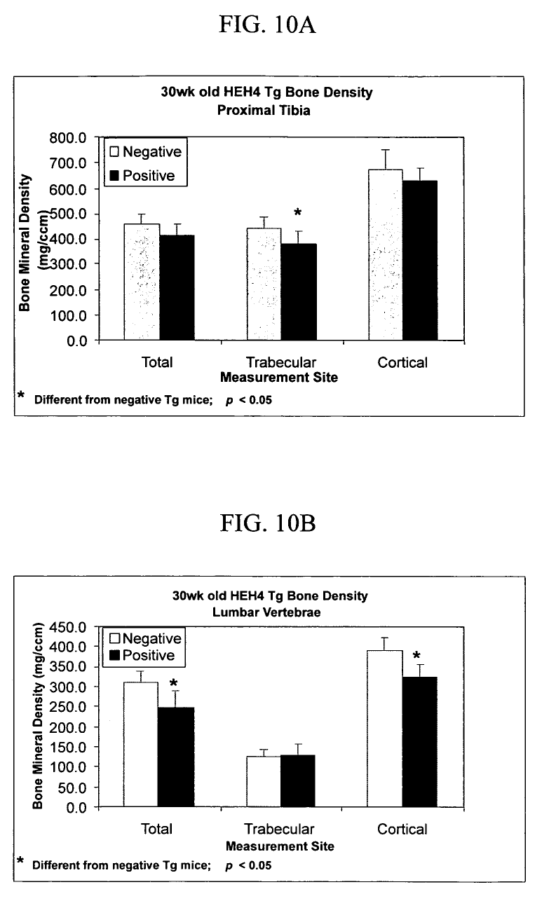

X-rays were taken of eight transgenic founder and four non-transgenic littermate controls at 30 weeks of age. A slight decrease was detected in the radiographic signal in the vertebrae of the transgenic mice. FIGS. 10A-10B show the results of peripheral quantitative computed tomography (PQCT) analysis, which confirmed the above observation. In addition, pQCT analysis showed that while the cortical bone density and total bone mineral density (BMD) in the tibia of transgenic and control mice were similar, the trabecular BMD in tibia samples was lower in transgenic versus control mice. pQCT analysis also showed a decrease in total bone mineral density (BMD) in the vertebrae of transgenic versus control mice. This decrease seems to be due to a decrease in cortical density in the vertebral bodies.

In general, the decrease in bone mineral density (BMD) obtained by pQCT analysis was consistant with the relative HEH4 expression levels detected in bone by Northern blot analysis. Specifically, the correlation between the highest transgene expression levels and the lowest measurements of bone mineral density, clearly indicates that increasing HEH4 levels/activity in bone in vivo results in a decrease in bone mineral density.

### EXAMPLE 4

### Production of Antibodies to HEH4 Polypeptides

Antibodies to HEH4 polypeptides may be obtained by immunization with purified protein (such as HEH4 polypeptides) or with, for example, HEH4 peptides produced by biological or chemical synthesis. Additionally, the HEH4 polypeptides or HEH4 peptides may be conjugated to a carrier protein that is immunogenic in the species to be immunized, such as keyhole limpet hemocyanin, serum, albumin, bovine thyroglobulin, or soybean trypsin inhibitor. Also, aggregating agents such as alum can be used to enhance the immune response. Suitable procedures for generating antibodies are know in the art, and include those described in Hudson and Hay, Practical Immunology, 2nd Edition, Blackwell Scientific Publications (1980). In one procedure for the production of antibodies, animals (typically mice or rabbits) are injected with a HEH4 antigen (such as a HEH4 polypeptide), and those with sufficient serum titer levels as determined by ELISA are selected for hybridoma production. Spleens of immunized animals are collected and prepared as single-cell suspensions from which splenocytes are recovered. The splenocytes are fused to mouse myeloma cells (such as Sp2/0-Ag14 cells; ATCC no. CRL-1581), allowed to incubate in DMEM with 200 U/ml penicillin, 200 μg/ml streptomycin sulfate and 4 mM glutamine, then incubated in HAT (Hypoxanthine; Aminopterin; Thymidine) selection medium. After selection, the tissue culture supernatants are taken from each fusion well and tested for anti-HEH4 antibody production by ELISA.

Alternative procedures for obtaining anti-HEH4 antibodies may also be employed, such as the immunization of transgenic mice harboring human Ig loci for the production of human antibodies, and the screening of synthetic antibody libraries, such as those generated by mutagenesis of an antibody variable domain. An additional alternative procedure for obtaining anti-HEH4 antibodies is to immunize a non-human “knock-out” animal (e.g., having a deletion, substitution or insertion within the HEH4 coding region, 5′ UTR, or promoter) in which the level of expression of HEH4 is significantly decreased or completely abolished. Animals such as these, that have very low or no endogenous production of HEH4 polypeptide, can mount a more desirable antibody response to HEH4 antigen than their wild type counterparts.

### EXAMPLE 5

### Screening Anti-HEH4 Antibodies to Identify Antagonists and Agonists of HEH4 Activity

Anti-HEH4 antibodies can be tested in appropriate cell-based assays to identify those antibodies which have HEH4 antagonistic or agonistic activity. One such type of cell-based assay involves differentiating appropriate cells (such as ST-2, C3H10T1/2, MC3T3-E1, MG-63 cells; or bone marrow derived mesenchymal stem cells from mice, rats or humans) down the osteoblast lineage using various agents (such as ascorbic acid, B-glycerophosphate, dexamethasone, BMPs, conditioned media containing Wnt activity or the like) alone or in various combinations, and then measuring markers, such as alkaline phosphatase or the accumulation of calcium (mineralization) of osteoblast activity. In such cell-based assays, addition of HEH4 polypeptide can inhibit differentiation (i.e., the measured level of an osteoblast activity marker would be lower than in the absence of the HEH4 polypeptide). An anti-HEH4 antagonistic antibody would thus be able to relieve the inhibition of differentiation caused by the addition of HEH4 polypeptide and thus the measured level of an osteoblast activity marker would be higher in the presence of the antagonistic antibody than in its absence. Conversely, an anti-HEH4 agonistic antibody would be able to further increase the inhibition of differentiation caused by the addition of HEH4 polypeptide, and thus the measured level of an osteoblast activity marker would be lower in the presence of the agonistic antibody than in its absence.

While the present invention has been described in terms of the preferred embodiments, it is understood that variations and modifications will occur to those skilled in the art. Therefore, it is intended that the appended claims cover all such equivalent variations that come within the scope of the invention as claimed.

1. A method for treating, preventing, or ameliorating a bone-related or cartilage-related disease disease, condition, or disorder, or modulating bone mineral density or bone strength in an individual, comprising administering to a patient an effective amount of an antibody or fragment thereof that specifically binds a polypeptide comprising:
(a) an amino acid sequence as set forth in any of SEQ ID NO: 2, SEQ ID NO: 4, or SEQ ID NO: 5;
(b) an amino acid sequence for an ortholog of any of SEQ ID NO: 2, SEQ ID NO: 4, or SEQ ID NO: 5; or
(c) an amino acid sequence for an allelic variant or splice variant of the amino acid sequence as set forth in any of SEQ ID NO: 2, SEQ ID NO: 4, or SEQ ID NO: 5, or the amino acid sequence of either (a) or (b).

2. The method of claim 1, wherein the bone-related disease, condition, or disorder is osteoporosis or osteopenia.

3. The method of claim 1, wherein the cartilage-related disease, condition, or disorder is osteoarthritis, dejenerative joint disease, or rheumatoid arthritis.

4. The method of claim 1, wherein the antibody or fragment thereof antagonizes HEH4 polypeptide biological activity.

5. The method of claim 1, wherein the antibody or fragment thereof agonizes HEH4 polypeptide biological activity.

6. The method of claim 1, wherein the antibody or fragment thereof is a humanized antibody or fragment thereof.

7. The method of claim 1, wherein the antibody or fragment thereof is a human antibody or fragment thereof.

8. The method of claim 1, wherein the antibody or fragment thereof is a polyclonal antibody or fragment thereof.

9. The method of claim 1, wherein the antibody or fragment thereof is a monoclonal antibody or fragment thereof.

10. The method of claim 1, wherein the antibody or fragment thereof is a chimeric antibody or fragment thereof.

11. The method of claim 1, wherein the antibody or fragment thereof is a CDR-grafted antibody or fragment thereof.

12. The method of claim 1, wherein the antibody or fragment thereof is an antiidiotypic antibody or fragment thereof.

13. The method of claim 1, wherein the antibody or fragment thereof is an antibody variable region fragment.

14. The method of claim 13, wherein the variable region fragment is a Fab or a Fab′ fragment.

15. The method of claim 1, wherein the antibody or fragment thereof comprises at least one antibody complementarity determining region with specificity for a polypeptide having the amino acid sequence of any of SEQ ID NO: 2, SEQ ID NO: 4, or SEQ ID NO: 5.

16. The method of claim 1, wherein the antibody or fragment thereof is bound to a detectable label.

17. A method for treating, preventing, or ameliorating osteoporosis or osteopenia comprising administering to a patient an effective amount of an antibody or fragment thereof that specifically binds a polypeptide comprising an amino acid sequence as set forth in SEQ ID NO: 4, wherein the antibody antagonizes HEH4 polypeptide biological activity.

18. A transgenic non-human mammal that is a HEH4 knock-out.

19. A process for obtaining an anti-HEH4 antibody comprising immunizing the transgenic mammal of claim 18 with an amino acid sequence as set forth in any of SEQ ID NO: 2, SEQ ID NO: 4, or SEQ ID NO: 5 or a fragment thereof.


SEQUENCE LISTING (7 sequences)

SEQ ID NO: 1 - DNA, 2310 bp, Homo sapiens
  CDS (37)..(1365)
gccgagtgggacaaagcctggggctgggcgggggccatggcgctgccatcccgaatcctgctttggaaacttgtgcttctgcagagctctgctgttctcctgcactcagggtcctcggtacccgccgctgctggcagctccgtggtgtccgagtccgcggtgagctgggaggcgggcgcccgggcggtgctgcgctgccagagcccgcgcatggtgtggacccaggaccggctgcacgaccgccagcgcgtgctccactgggacctgcgcggccccgggggtggccccgcgcggcgcctgctggacttgtactcggcgggcgagcagcgcgtgtacgaggcgcgggaccgcggccgcctggagctctcggcctcggccttcgacgacggcaacttctcgctgctcatccgcgcggtggaggagacggacgcggggctgtacacctgcaacctgcaccatcactactgccacctctacgagagcctggccgtccgcctggaggtcaccgacggccccccggccacccccgcctactgggacggcgagaaggaggtgctggcggtggcgcgcggcgcacccgcgcttctgacctgcgtgaaccgcgggcacgtgtggaccgaccggcacgtggaggaggctcaacaggtggtgcactgggaccggcagccgcccggggtcccgcacgaccgcgcggaccgcctgctggacctctacgcgtcgggcgagcgccgcgcctacgggcccctttttctgcgcgaccgcgtggctgtgggcgcggatgcctttgagcgcggtgacttctcactgcgtatcgagccgctggaggtcgccgacgagggcacctactcctgccacctgcaccaccattactgtggcctgcacgaacgccgcgtcttccacctgacggtcgccgaaccccacgcggagccgcccccccggggctctccgggcaacggctccagccacagcggcgccccaggcccagaccccacactggcgcgcggccacaacgtcatcaatgtcatcgtccccgagagccgagcccacttcttccagcagctgggctacgtgctggccacgctgctgctcttcatcctgctactggtcactgtcctcctggccgcccgcaggcgccgcggaggctacgaatactcggaccagaagtcgggaaagtcaaaggggaaggatgttaacttggcggagttcgctgtggctgcaggggaccagatgctttacaggagtgaggacatccagctagattacaaaaacaacatcctgaaggagagggcggagctggcccacagccccctgcctgccaagtacatcgacctagacaaagggttccggaaggagaactgcaaatagggaggccctgggctcctggctgggccagcagctgcacctctcctgtctgtgctcctcggggcatctcctgatgctccggggctcaccccccttccagcggctggtcccgctttcctggaatttggcctgggcgtatgcagaggccgcctccacacccctcccccaggggcttggtggcagcatagcccccacccctgcggcctttgctcacgggtggccctgcccacccctggcacaaccaaaatcccactgatgcccatcatgccctcagacccttctgggctctgcccgctgggggcctgaagacattcctggaggacactcccatcagaacctggcagccccaaaactggggtcagcctcagggcaggagtcccactcctccagggctctgctcgtccggggctgggagatgttcctggaggaggacactcccatcagaacttggcagccttgaagttggggtcagcctcggcaggagtcccactcctcctggggtgctgcctgccaccaagagctcccccacctgtaccaccatgtgggactccaggcaccatctgttctccccagggacctgctgacttgaatgccagcccttgctcctctgtgttgctttgggccacctggggctgcaccccctgccctttctctgccccatccctaccctagccttgctctcagccaccttgatagtcactgggctccctgtgacttctgaccctgacacccctcccttggactctgcctgggctggagtctagggctggggctacatttggcttctgtactggctgaggacaggggagggagtgaagttggtttggggtggcctgtgttgccactctcagcaccccacatttgcatctgctggtggacctgccaccatcacaataaagtccccatctgatttttaaaaaaaaaaaaaaaaaaaaaaaaaaaaaaaaa

SEQ ID NO: 2 - protein, 442 aa, Homo sapiens
MALPSRILLWKLVLLQSSAVLLHSGSSVPAAAGSSVVSESAVSWEAGARAVLRCQSPRMVWTQDRLHDRQRVLHWDLRGPGGGPARRLLDLYSAGEQRVYEARDRGRLELSASAFDDGNFSLLIRAVEETDAGLYTCNLHHHYCHLYESLAVRLEVTDGPPATPAYWDGEKEVLAVARGAPALLTCVNRGHVWTDRHVEEAQQVVHWDRQPPGVPHDRADRLLDLYASGERRAYGPLFLRDRVAVGADAFERGDFSLRIEPLEVADEGTYSCHLHHHYCGLHERRVFHLTVAEPHAEPPPRGSPGNGSSHSGAPGPDPTLARGHNVINVIVPESRAHFFQQLGYVLATLLLFILLLVTVLLAARRRRGGYEYSDQKSGKSKGKDVNLAEFAVAAGDQMLYRSEDIQLDYKNNILKERAELAHSPLPAKYIDLDKGFRKENCK

SEQ ID NO: 3 - DNA, 2144 bp, Rattus norvegicus
  CDS (43)..(1371)
gagctgagtaagacaaagcacggtgcagtccccgcaggcgccatggaactgctatcccgtgtcttgttgtggaaactggtgcttcttcagagttctgcagtcctgtcctcagggtctccagggaccgcagcagccagcagctctgtggtgtctgagtctgcggtgagctgggcagccggaactcaggcggtgctacgctgccagagcccgcgcatggtgtggacccaagaccggctgcacgatcgccagcgcgtggtccactgggacctcagcggtggcccaggtagccaagggcgccgacttgtggatatgtactcggccggggaacagcgcgtgtaccagccgcgcgatcgcgaccgcctcctgctgtcgccttctgccttccacgacggcaacttctcgctgctcatccgcgctgtggagagaggcgacgaaggggtgtacacctgcaatctgcaccatcactactgccacctctacgagagcctggctgtgcgcctcgaggtcactgacgatcccctattaagtcgcgcatactgggacggagagaaggaagtgttggtggtggccctcggcgcaccggctctgatgacttgcgtgaaccgtgagcacttgtggactgaccgccacttagaggaggcgcagcaggtggtccattgggaccgacagctacctggcgtgccacatgaccgcgctgaccgattgcttgacctgtacgcatccggcgagcgccgagcctacgggccacccttcctgcgtgatcgcgtgtcggtgaacaccaacgcttttgcacgcggggacttctccctacgcatagatgacctggagccggctgatgagggcatctattcctgccacctgcaccaccactactgtggtctccatgagcgccgagtcttccacctacgggtcacggagcctgtttttgagccaccagctcgcgcttctcctggcaatgggtctggtcacaacagtgttcctagcccagatcccaccatggcccgtggccacagcatcatcaacgtcattgtcccagaggaccacacacatttcttccagcaattgggctatgtgctggccactctactgcttttcatcttgctgctcatcactgtagtcctggcgacacgccaccgtcacagtggaggatgcaagacctcagacaggaaagctgggaaatcaaaggggaaggatgtgaacatgatggagtttgctatagccacaagggaccaggctccgtataggactgaggacatccagctagattacaaaaataacatcctgaaggagagggctgggctggcccacagtcccctgcctgccaaggacgtggatctggataaagagttcaggaaggagtactgcaaataaatggaccctgagcttctggctgggccagcagctctgcatcaaaggacatcttcctgaccctcctgtggtattcctggctcttctcaacggctggtccggcttacctagagacttggcttaaacttggcagagcagctgcctgtactttgctcttcctagaatcacccccctcatgttggtgagcaactgtgggttccctagggactctggtatagtatcattgctgcccttcaatcacctgtgcccactgggggctgtaccccaacttcaacacagcaaaaaatccctcattaatatccaccaaacgcaaagtcccccgtggcctcttactgctaggatcgggaagacactaacggattccagttaagactcctagcccctagttaaacacattaaccactgtcctggtaagactgcactacgcctgtgcactgctcaggggcctgctgtcatgctctgactcgtggccctccttgctgctttgggcaacttggggcaggcccatggccctttctctgcttctgattcctttctgtccaatgctctccaagagccacaccaacagttactggggaccatgtgactcttagtcttcatatcacttccctaggctggagtctgggattggggtcccatttgtgctcttttttgactgaggatgggatgaagagggatctgaggagcccatggtgcacatcaaacagctatcatttgctcctactggggaagttctcatgtgataataaaagatatatctgaaaaaaaaaaaaaaa

SEQ ID NO: 4 - protein, 442 aa, Rattus norvegicus
MELLSRVLLWKLVLLQSSAVLSSGSPGTAAASSSVVSESAVSWAAGTQAVLRCQSPRMVWTQDRLHDRQRVVHWDLSGGPGSQGRRLVDMYSAGEQRVYQPRDRDRLLLSPSAFHDGNFSLLIRAVERGDEGVYTCNLHHHYCHLYESLAVRLEVTDDPLLSRAYWDGEKEVLVVALGAPALMTCVNREHLWTDRHLEEAQQVVHWDRQLPGVPHDRADRLLDLYASGERRAYGPPFLRDRVSVNTNAFARGDFSLRIDDLEPADEGIYSCHLHHHYCGLHERRVFHLRVTEPVFEPPARASPGNGSGHNSVPSPDPTMARGHSIINVIVPEDHTHFFQQLGYVLATLLLFILLLITVVLATRHRHSGGCKTSDRKAGKSKGKDVNMMEFAIATRDQAPYRTEDIQLDYKNNILKERAGLAHSPLPAKDVDLDKEFRKEYCK

SEQ ID NO: 5 - protein, 442 aa, Mus musculus
MELLSRVLLWKLLLLQSSAVLSSGPSGTAAASNSLVSESVVSLAAGTQAVLRCQSPRMVWTQDRLHDRQRVVHWDLSGDPGSQRRRLVDMYSAGEQRVYEPRDRDRLLLSPSAFHDGNFSLLIRAVERGDEGVYTCNLHHHYCHLDESLAVRLEVTDDPLLSRAYWDGEKEVLVVAHGAPALMTCINRAHVWTDRHLEEAQQVVHWDRQLPGVSHDRADRLLDLYASGERRAYGPPFLRDRVSVNTNAFARGDFSLRIDELERADEGIYSCHLHHHYCGLHERRVFHLQVTEPAFEPPARASPGNGSGHSSAPSPDPTLTRGHSIINVIVPEDHTHFFQQLGYVLATLLLFILLLITVVLATRHRHSGGCKTSDKKAGKSKGKDVNMVEFAVATRDQAPYRTEDIQLDYKNNILKERAELAHSPLPAKDVDLDKEFRKEYCK

SEQ ID NO: 6 - protein, 11 aa, Human immunodeficiency virus type 1
YGRKKRRQRRR

SEQ ID NO: 7 - protein, 15 aa, artificial
  internalizing domain derived from HIV tat protein
GGGGYGRKKRRQRRR